(19) United States
(12) Patent Application Publication
(10) Pub. No.: US 2025/0352774 A1
(43) Pub. Date: Nov. 20, 2025

(54) BALLOON CATHETER AND METHODS OF USING THE SAME

(71) Applicant: Airiver Medical, Inc., Dover, NV (US)

(72) Inventors: Lixiao Wang, Henderson, NV (US); Paul Anthony Vajgrt, St. Michael, MN (US); Mitchell Robert Hovaness Erikson, Crystal, MN (US); Yan Li, Lino Lakes, MN (US)

(21) Appl. No.: 19/281,082

(22) Filed: Jul. 25, 2025

Related U.S. Application Data
(63) Continuation-in-part of application No. 18/709,120, filed on May 10, 2024.
(60) Provisional application No. 63/279,776, filed on Nov. 16, 2021.

(51) Int. Cl. A61M 25/10

(52) U.S. Cl. CPC A61M 25/1002; A61M 2025/105

(57) ABSTRACT

Methods of treating a central airway obstruction (CAO), benign airway stenosis, asthma, chronic obstructive pulmonary disease (COPD), or an airway tumor in a human subject, methods of treating chronic rhinosinusitis (CRS) or chronic rhinosinusitis with nasal polyps (CRSwNP) in a human subject, methods of treating a eustachian tube disorder in a human subject, and devices and systems for performing the methods. A method of treating a central airway obstruction (CAO), benign airway stenosis, asthma, chronic obstructive pulmonary disease (COPD), or an airway tumor in a human subject includes inserting a drug-coated balloon catheter into a target site in a trachea, mainstem bronchus, bronchus intermedius, lobar bronchus, segmental bronchus, or sub-segmental bronchus, wherein the balloon is coated with a coating layer including the therapeutic agent and one or more additives. The therapeutic agent includes paclitaxel, sirolimus, or a derivative thereof. The method includes inflating the balloon to dilate the airway and deliver a therapeutic agent to the airway wall. The method includes deflating the balloon. The method also includes withdrawing the balloon catheter from the target site.

### CROSS-REFERENCE TO RELATED APPLICATION

This application is a continuation-in-part of and claims the benefit of priority under 35 U.S.C. § 120 to U.S. Utility Application Ser. No. 18/709,120 filed May 10, 2024, which is a U.S. National Stage Filing under 35 U.S.C. 371 from International Application No. PCT/US2022/050087 filed Nov. 16, 2022, which claims the benefit of priority to U.S. Provisional Patent Application Ser. No. 63/279,776 filed Nov. 16, 2021, the disclosures of which is incorporated herein in its entirety by reference.

### BACKGROUND

Chronic rhinosinusitis (CRS) is an inflammation of the membrane lining of one or more paranasal sinuses. Chronic rhinosinusitis lasts longer than three weeks and often continues for months. In cases of chronic rhinosinusitis, there is usually tissue damage. According to the Center for Disease Control, thirty-seven million cases of chronic sinusitis are reported annually.

Chronic rhinosinusitis (CRS) is widely thought to be a common disease—many studies cite a prevalence of 10-15% (see, e.g., Adam S. DeConde et al., Am J Rhinol Allergy 30, 134-139, 2016). Chronic rhinosinusitis with Nasal Polyps (CRSwNP) and Nasal Polyps (NP) is about 25-30% of CRS. CRSwNP is an inflammatory condition of the nose and paranasal sinuses of unknown cause which is present in 2%-4% of the adult population. In the USA, the direct costs for the management of CRS are now between $10 and $13 billion per year, or about $2.6K per patient per year. The highest direct costs were associated with patients who had recurrent polyposis after surgery. The surgery is expensive, varying from up to $11,000 in USA (EPOS 2020). Surgery and medication are recommended for difficult-to-treat CRS (patients who have persistent symptoms of CRS despite appropriate treatment). Recurrence of CRSwNP after surgery is common and occurs in as many as 60% of patients (50% of these patients have had previous surgery) (see, Adam S. DeConde et al., Am J Rhinol Allergy 30, 134-139, 2016).

Asthma is a chronic respiratory disease characterized by inflammation of the airways, excess mucus production, airway hyper-responsiveness, and a condition in which airways narrow excessively or too easily respond to a stimulus. Asthma episodes or attacks cause narrowing of the airways via smooth muscle contractions in the airways, which makes breathing difficult. Asthma attacks can have a significant impact on a patient's life and can limit participation in many activities. In severe cases, asthma attacks can be life-threatening. Of the more than 35 million people in the U.S. living with asthma, about 5-10% suffer from severe asthma. Presently, there is no known cure for asthma.

CRS is associated with asthma, with a prevalence of asthma around 25% in patients with CRS compared to 5% in the general population. Patients with chronic rhinosinusitis with nasal polyps (CRSwNP) often have coexisting asthma under the concept of “United Airway Disease”, being the combination of both diseases, which is one of the most challenging disease to treat. The effect of CRSwNP treatment, whether medical or surgical, in asthma is today less controversial after some studies have shown improvement of asthma after medical and/or surgical treatment of CRSwNP.

Chronic obstructive pulmonary disease (COPD) is a term used to classify two major airflow obstruction disorders: chronic bronchitis and emphysema. Approximately 16 million Americans have COPD, with about 80-90% of them smokers throughout much of their lives. COPD is a leading cause of death in the U.S. Chronic bronchitis is inflammation of the bronchial airways. The bronchial airways connect the trachea with the lungs. When inflamed, the bronchial tubes secrete mucus, causing a chronic cough. Emphysema is an over-inflation of the alveoli, or air sacs in the lungs. This condition causes shortness of breath.

Mucus can accumulate in the lungs and can plug up the airway, reducing air flow. Airway plugs can include viscous high molecular weight glycoproteins. These proteins are over-produced by goblet cells and submucosal glands in the airway tracts of the lungs. If the mucus plugs are in the larger upper airways, such as mainstem bronchus, bronchus intermedius, or a lobar bronchus, this condition can lead to a shortness of breath or death of the patients as seen in chronic bronchitis, asthma, and cystic fibrosis.

The uncontrolled localized production of IL-5, IL-6, IL-4, IL-13, IL-23, and/or ILC2 in the airway can contribute to airway eosinophilia in patients with severe eosinophilic asthma. IL-5, IL-6, IL-4, IL-13, IL-23, eosinophils, and other targeted cells in airway can be responsible for airway inflammation in asthmatics. Various humanized monoclonal antibodies have been developed to target these cells to treat severe asthma. These biological medicines require continued administration yearly or throughout the patient's life, with the disease reoccurring upon cessation of treatment.

Airway restenosis is one of the challenging pathologies to treat in the field of otorhinolaryngology and pulmonology. It can be at the level of supraglottis, glottis, subglottis, trachea, mainstem bronchus, bronchus intermedius, lobar bronchus, segmental bronchus, or sub-segmental bronchus. Of the wide range of etiologies, e.g., congenital, traumatic, inflammatory, and idiopathic, trauma following prolonged intubation and tracheostomy is still considered the commonest cause for the development of airway stenosis in both pediatric and adult population. Benign strictures or restenosis constitute most benign forms of airway restenosis and include airway restenosis related to post-intubation tracheal stenosis, post-tracheostomy tracheal restenosis, post-tuberculosis infection, transplant-related, and idiopathic restenosis. There are various methods for alleviating symptoms in patients with airway restenosis, which include mechanical debulking, rigid bronchoscopic dilation, stent placement, and balloon dilation. These treatments can be used to provide relief of the stenotic or strictured airway segment, though the stenosis often recurs and then repeated treatments are needed.

### SUMMARY OF THE INVENTION

The present invention provides a method of treatment of a recurring airway stricture or stenosis in an airway body lumen. The method includes inserting a scope and a balloon catheter into the target site at the recurring airway stricture of stenosis in the airway body lumen. The balloon catheter includes an elongated balloon. The balloon catheter also includes a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site.

The present invention provides a method of treatment of a recurring airway stricture or stenosis in an airway body lumen. The method includes damaging, dilating, and/or removing the stricture or stenosis at a target site in the body lumen. The method includes flushing the target site with a flushing composition including water and/or saline. The method includes inserting a scope and a balloon catheter into the target site. The balloon catheter includes an elongated balloon. The balloon catheter also includes a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes hydrating and/or soaking the coating in the flushing composition at the target site. The method includes inflating the balloon to an inflation diameter such that the coating contacts an interior of the body lumen at the target site. The method includes deflating the balloon. The method also includes withdrawing the scope and the balloon catheter from the target site. Prior to inflation the coating on the drug coated balloon is hydrated and/or soaked to activate the coating.

The present invention provides a method of treatment of chronic rhinosinusitis with nasal polyps (CRSwNP). The method includes removing at least one of the nasal polyps at a target site in the body lumen. The method includes inserting a scope and a balloon catheter into the target site in a body lumen. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. Prior to inflation the coating on the drug coated balloon is hydrated and/or soaked to activate the coating.

The present invention provides a method of treatment of laryngostenosis and/or subglottic stricture or stenosis. The method includes damaging, dilating, and/or removing the laryngostenosis and/or subglottic stricture or stenosis at a target site in the body lumen. The method includes inserting a scope and a balloon catheter into the target site in a body lumen. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. Prior to inflation the coating on the drug coated balloon is hydrated and/or soaked to activate the coating.

The present invention provides a method of treatment of severe asthma. The method includes inserting a scope and a balloon catheter into a target site in a body lumen including a trachea, mainstem bronchus, bronchus intermedius, lobar bronchus, segmental bronchus, or sub-segmental bronchus. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. The method can include treating more than one treatment site. The method can include at least one drug-coated balloon catheter, two drug-coated balloon catheters, or more than two drug-coated balloon catheters. In aspects that include treating more than one treatment site, the treatments can be performed sequentially or a time delay can occur between the treatments, such as a time delay of 1 to 6 weeks. For example, in one aspect, bronchi in the upper lung can be treated in a first treatment, and untreated bronchi in a different part of the lung can be treated in a later (e.g., second) treatment. Asthma episodes or attacks can cause narrowing of the airways by smooth muscle contractions in the airways. In various aspects, the drug released from the drug-coated balloon can reduce the amount of smooth muscle cells in the airway, such as by 5% to 50%. Prior to inflation the coating on the drug coated balloon is hydrated and/or soaked to activate the coating.

The present invention provides a method of reduction of exacerbation of and/or hospitalization for severe asthma. The method includes inserting a scope and a balloon catheter into a target site in a body lumen including a trachea, mainstem bronchus, bronchus intermedius, lobar bronchus, segmental bronchus, or sub-segmental bronchus. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. In various aspects, the drug released from the drug-coated balloon can reduce the concentration of smooth muscle cells in the airway, such as by 5% to 50%, leading to reduction of exacerbation of and/or hospitalization for severe asthma and less smooth muscle contractions in the airway. Prior to inflation the coating on the drug coated balloon is hydrated and/or soaked to activate the coating.

The present invention provides a method of reducing a concentration of bronchial smooth muscle cells of a severe asthma patient. The method includes inserting a scope and a balloon catheter into a target site including bronchial smooth muscle cells in a body lumen. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. Prior to inflation the coating on the drug coated balloon is hydrated and/or soaked to activate the coating.

The present invention provides a method of treatment of chronic rhinosinusitis with nasal polyps (CRSwNP) and asthma. The method includes removing at least one of the nasal polyps at a first target site in a first body lumen. The method includes inserting a first scope and a first balloon catheter into the first target site in the first body lumen. The first balloon catheter includes a first elongated balloon. The first balloon catheter also includes a first coating layer overlying an exterior surface of the first balloon. The first coating layer includes one or more first additives and an initial drug load of a first therapeutic agent chosen from paclitaxel, docetaxel, taxol, rapamycin, sirolimus, zotarolimus, everolimus, tacrolimus, an analogue thereof, and a combination thereof. The method includes inflating the first balloon to a first inflation diameter such that the first coating layer contacts an interior of the first body lumen at the first target site. The method includes deflating the first balloon. The method includes withdrawing the first scope and the first balloon catheter from the first target site. The method includes inserting a second scope and a second balloon catheter into a second target site in a second body lumen including a trachea and/or a bronchus. The second balloon catheter includes a second elongated balloon. The second balloon catheter also includes a second coating layer overlying an exterior surface of the second balloon. The second coating layer includes one or more second additives and an initial drug load of a second therapeutic agent chosen from paclitaxel, docetaxel, taxol, rapamycin, sirolimus, zotarolimus, everolimus, tacrolimus, an analogue thereof, and a combination thereof. The method includes inflating the second balloon to an inflation diameter such that the coating contacts an interior of the second body lumen at the second target site. The method includes deflating the second balloon. The method also includes withdrawing the second scope and the second balloon catheter from the second target site. In various aspects, the second target site includes a bronchus, such as a mainstem bronchus, bronchus intermedius, and/or a lobar bronchus. In various aspects, the asthma is severe asthma. In various aspects, treating CRSwNP and asthma simultaneously can advantageously cause synergism with greater total treatment effect that the combined total treatment effect of treating CRSwNP and asthma independently. Prior to inflation the coating on the first and/or second drug coated balloon is hydrated and/or soaked to activate the coating.

The present invention provides a method of reducing a concentration of eosinophils, IL-4, IL-5, IL-6, IL-13, IL-23, and/or ILC2 in an airway tract, such as in severe asthma patients. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. Prior to inflating the coating on the drug coated balloon is hydrated and/or soaked to activate the coating.

The present invention provides a method of treatment of a mucous plug in an airway. The method can optionally include removing the mucous plug. The method includes inserting a scope and a balloon catheter into a target site that included a mucous plug in an airway body lumen. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. In various aspects, the drug released from the drug-coated balloon can reduce the concentration of goblet cells in the airway, preventing or reducing production of viscous mucous plugs in the airway. Prior to inflation the coating on the drug coated balloon is hydrated and/or soaked to activate the coating.

The present invention provides a method of reduction of concentration of goblet cells in an airway tract. The method includes inserting a scope and a balloon catheter into a target site including one or more goblet cells in a body lumen. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. The drug released from the drug coated balloon can reduce the concentration of goblet cells in the airway. Prior to inflation the coating on the drug coated balloon is hydrated and/or soaked to activate the coating.

The present invention provides a method of reducing a concentration of one or more mucins in the airway tract. Mucins are gel-forming glycoproteins in mucous plugs. Mucins are produced by mucous overproduction and hypersecretion in chronically inflamed airways. The method includes inserting a scope and a balloon catheter into a target site in a body lumen including a trachea, mainstem bronchus, bronchus intermedius, lobar bronchus, segmental bronchus, or sub-segmental bronchus. The balloon catheter includes an elongated balloon and a coating layer overlying an exterior surface of the balloon. The coating layer includes one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method includes inflating the balloon to an inflation diameter such that the coating contacts an interior of the body lumen at the target site. The method includes deflating the balloon. The method also includes withdrawing the scope and the balloon catheter from the target site. Prior to inflation the coating on the drug coated balloon is hydrated and/or soaked to activate the coating.

In various aspects, the present invention provides a minimally invasive method for treatment or prevention of recuring stricture or stenosis of the frontal sinus, ethmoid sinus, sphenoid sinus, maxillary sinus, nasal passage, supraglottis, glottis, subglottis, trachea, mainstem bronchus, bronchus intermedius, lobar bronchus, segmental bronchus, or sub-segmental bronchus. The method includes inserting a catheter with an expandable body through the body lumen that contains the recurring stricture or stenosis such that the expandable body is inside the stricture or stenosis. The catheter with an expandable body can include a balloon catheter, a drug coated catheter, a drug eluting stent, and/or a drug eluting stent crimped on a drug coated balloon. The catheter includes an expandable body with a coating layer, or more than one layer, containing a therapeutic agent and one more additives overlying the exterior surface of the expandable body of the catheter. The one or more additives are chosen from one or more water insoluble additives, slightly water insoluble additives, partially water soluble additives, water soluble additives, or a combination thereof. The method includes expanding the body to contact the coating layer with the stricture or stenosis in the body lumen to a certain diameter for a period of time. The method includes contracting the expanded body after the time period and withdrawing the catheter from the treated stricture or stenosis. In some aspects, the method further includes performing a surgical procedure to dilate, cut, or remove tissue prior to the insertion of the balloon catheter into the target site. Prior to inflation the coating on the drug coated balloon is hydrated and/or soaked to activate the coating.

Aspects of the present invention provide a medical device coating formulation including a therapeutic agent or drug for treatment of the strictures or stenosis in airway body lumens, and additives that enhance absorption of the drug into tissue of body lumens. Some aspects provide a coating that overlays the expandable portion of the catheter that has a single layer or multiple layers that contain a single or multiple therapeutic agent. In some aspects the layer in contact with the expandable portion of the catheter has no therapeutic agent and is formulated with ingredients that allow the entire or a substantial portion of the coating to transfer to the stricture or stenosis upon expansion of the catheter. Causes of the stricture or stenosis can include infections and inflammations by pathogens such as bacteria and viruses. In some aspects the coating has additives that have antibacterial and antiviral properties. In some aspects the layer of coating that contains the therapeutic agent includes drug that is crystalline, amorphous, or a combination thereof. In some aspects the layer of coating that contains the therapeutic agent has at least one hydrophilic ingredient and at least one hydrophobic ingredient. In some aspects the coating layers contain ingredients that enhance the adhesion of the coating with the luminal surface of the dilated stricture or stenosis. In some aspects the coating is formulated such that upon expansion of the catheter the coating transfers to the stricture or stenosis as a particulate, agglomerated particulate, dissolved matter, or a combination thereof. In some aspects the size of the particulate or agglomerated particulate transferred is small and less than 10 μm, or more preferably less than 5 μm.

In various aspects, the present invention provides a catheter including an expandable portion of an elongated body that is used to dilate recuring airway strictures or restenosis. In some aspects the elongated body is a balloon with a cylindrical shape. In some aspects the elongated body is a balloon that has a shape or longitudinal profile that prevents migration of the balloon in the body lumen it is expanded in.

Aspects of the present invention relate to balloon catheters having a rapid drug-releasing coating and methods for using the same to treat airway strictures or stenosis. The therapeutic agent according to aspects of the present invention does not require a delayed or long-term release; rather, the therapeutic agent and the additive are released in a short time period to provide a rapid therapeutic effect. An object of aspects of the present invention is to facilitate rapid and efficient uptake of drug by target tissue during transitory device deployment at a target site.

Various aspects of the present invention provide a method of treating a central airway obstruction (CAO), benign airway stenosis, asthma, chronic obstructive pulmonary disease (COPD), or an airway tumor in a human subject. The method includes inserting a drug-coated balloon catheter into a target site in a trachea, mainstem bronchus, bronchus intermedius, lobar bronchus, segmental bronchus, or sub-segmental bronchus. The balloon is coated with a coating layer including a therapeutic agent including paclitaxel, sirolimus, or a derivative thereof, and one or more additives. The method includes inflating the balloon to dilate the airway and deliver the therapeutic agent to the airway wall. The method includes deflating the balloon. The method also includes withdrawing the balloon catheter from the target site.

Various aspects of the present invention provide a method of treating chronic rhinosinusitis (CRS) or chronic rhinosinusitis with nasal polyps (CRSwNP) in a human subject. The method includes identifying a target lesion in a paranasal sinus or sinus drainage pathway. The method optionally includes performing surgical dissection, irrigation, or balloon dilation with an uncoated balloon. The method includes inserting a drug-coated balloon catheter into the target lesion. The balloon is coated with a coating layer including a therapeutic agent and one or more additives. The therapeutic agent includes paclitaxel, sirolimus, or a derivative thereof. The method includes inflating the balloon to dilate the lesion and deliver a therapeutic agent to the tissue. The method includes deflating the balloon. The method also includes withdrawing the balloon catheter.

Various aspects of the present invention provide a method of reshaping and flattening out residual bony partitions in a surgically resected ethmoid sinus cavity. The method includes performing surgical ethmoidectomy to create a resected ethmoid cavity. The method includes inserting a balloon catheter into the resected ethmoid cavity. The method includes inflating the balloon to reshape and flatten residual bony partitions. The method also includes deflating and removing the balloon.

Various aspects of the present invention provide a method of treating a eustachian tube disorder in a human subject. The method includes inserting a balloon catheter into the eustachian tube. The balloon is coated with a coating layer including a therapeutic agent and one or more additives. The therapeutic agent includes paclitaxel, sirolimus, or a derivative thereof. The method includes inflating the balloon to dilate the eustachian tube and deliver a therapeutic agent to the tissue. The method includes deflating the balloon. The method also includes withdrawing the balloon catheter.

Various aspects of the present invention provide a device for treating nasal sinuses or paranasal sinus drainage pathways. The device includes a tubular balloon catheter with an integrated inflation lumen. The device includes a hollow region under the balloon. The device also includes an access port in the distal portion of the device but proximal to the balloon, through which a positioning instrument may be inserted.

Various aspects of the present invention provide a system including a balloon dilation device. The balloon dilation device includes a tubular balloon catheter with an integrated inflation lumen. The balloon dilation device includes a hollow region under the balloon. The balloon dilation device also includes an access port in the distal portion of the device but proximal to the balloon, through which a positioning instrument may be inserted. The system also includes a positioning instrument configured to be inserted into the balloon device through the access port in the distal portion of the device but proximal to the balloon.

Various aspects of the present invention provide a method of positioning a balloon catheter in a nasal cavity, paranasal sinus, or sinus drainage pathway. The method includes inserting a positioning instrument into a port in the distal end of the balloon catheter, proximal to the balloon. The method also includes positioning the balloon catheter and positioning instrument into the nasal cavity and directing the balloon catheter to the target location.

Various conventional treatments for severe asthma or chronic rhinosinusitis with nasal polyps (CRSwNP) require yearly retreatments or continued retreatments throughout a patient's life to avoid recurrence of the treated condition. However, in various aspects of the present invention, the method can be effective to avoid recurrence of the treated condition, such as for the patient's entire life, or for 3 years, 5 years, or 10 years.

### BRIEF DESCRIPTION OF THE FIGURES

The drawings illustrate generally, by way of example, but not by way of limitation, various aspects of the present invention.

FIG. 1A illustrates a balloon catheter having one neck section, in accordance with various aspects of the present disclosure.

FIG. 1B illustrates a balloon catheter having two neck sections, in accordance with various aspects of the present disclosure.

FIG. 1C illustrates a balloon catheter having three neck sections, in accordance with various aspects of the present disclosure.

FIG. 2 illustrates a two-necked drug-coated balloon catheter including a catheter shaft, catheter tip, and Tuohy Borst adapter, in accordance with various aspects (the balloon catheter includes a fixed wire, over the wire, and rapid exchanged balloon catheters details not shown in FIG. 2), in accordance with various aspects of the present disclosure.

FIG. 3 is a perspective view of an aspect of a balloon catheter according to the present invention (the balloon catheter includes a fixed wire, over the wire, and rapid exchanged balloon catheters details not shown in FIG. 3), in accordance with various aspects of the present disclosure.

FIGS. 4A-4C are cross-sectional views of different aspects of the distal portion of the balloon catheter of FIG. 3, taken along line A-A, showing exemplary coating layers, in accordance with various aspects of the present disclosure.

FIG. 5 illustrates a drug-coated balloon catheter on a guidewire, in accordance with various aspects of the present disclosure.

FIG. 6 illustrates 1-h paclitaxel concentration in treated tissues, in accordance with various aspects of the present disclosure.

FIG. 7 illustrates concentration of paclitaxel versus time, in accordance with various aspects of the present disclosure.

FIG. 8A illustrates a test article treatment site 28 days after treatment, in accordance with various aspects of the present disclosure.

FIG. 8B illustrates a control article treatment site after 28 days, in accordance with various aspects of the present disclosure.

FIG. 9 illustrates percentage freedom from target lesion reintervention versus days, in accordance with various aspects of the present disclosure.

FIG. 10 illustrates the FEV₁ percent predicted distribution versus days post-procedure, in accordance with various aspects of the present disclosure.

FIG. 11 shows the mean FEV₁ (L) change versus days post-procedure, in accordance with various aspects of the present disclosure.

FIG. 12 illustrates FEV₁ MCID change from baseline versus days post-procedure, in accordance with various aspects of the present disclosure.

FIG. 13 illustrates mMRC dyspnea grade distribution versus days post-procedure, in accordance with various aspects of the present disclosure.

FIG. 14 illustrates mMRC dyspnea change from baseline versus days post-procedure, in accordance with various aspects of the present disclosure.

FIG. 15 illustrates the DCB with drug coating on the cylindrical area between the balloon cones, in accordance with various aspects of the present disclosure.

FIG. 16 illustrates the DCB with drug coating on the cylindrical area between the balloon cones and on the distal cone, in accordance with various aspects of the present disclosure.

FIG. 17 illustrates SNOT-22 scores for baseline through 12 months, in accordance with various aspects of the present disclosure.

FIG. 18 illustrates the percentage of subjects achieving MCID improvement in SNOT-22 and EQ-5D-VAS at timepoints 30-days through 1-year post-procedure, in accordance with various aspects of the present disclosure.

FIG. 19 illustrates the SNOT-22 Nasal Blockage (nasal congestion) score for 32 RESTORE-1 subjects through 12 months, in accordance with various aspects of the present disclosure.

FIG. 20 illustrates presence of polyps at the 6-month and 12-month endoscopies compared to published data for FESS alone, in accordance with various aspects of the present disclosure.

FIG. 21 illustrates the Lund-Mackay CT score for assessment of radiographic evidence of inflammation in each sinus and the ostiomeatal complex (OMC) for each side, in accordance with various aspects of the present disclosure.

FIG. 22 illustrates mean LMK-CT scores at baseline, 6-months, and 12-months post-procedure.

FIG. 23 illustrates a schematic representation of a balloon catheter device, in accordance with various aspects of the present disclosure.

FIG. 24 illustrates a schematic representation of the balloon catheter device of FIG. 23 showing the instrument insertion point, in accordance with various aspects of the present disclosure.

FIG. 25 illustrates a schematic representation of the balloon of the ballon catheter device of FIG. 23, in accordance with various aspects of the present disclosure.

FIG. 26 illustrates a detailed view of an open version of a tip of the balloon catheter device of FIG. 23, in accordance with various aspects of the present disclosure.

FIG. 27 illustrates a sectional view of a proximal catheter shaft, in accordance with various aspects of the present disclosure.

FIG. 28 illustrates an image of a balloon catheter device, in accordance with various aspects of the present disclosure.

FIG. 29 illustrates an image of the balloon catheter device of FIG. 28 showing the instrument access port, in accordance with various aspects of the present disclosure.

FIG. 30 illustrates a sinus seeker instrument, in accordance with various aspects of the present disclosure.

FIG. 31 illustrates a balloon catheter device inserted into a sinus seeker instrument, in accordance with various aspects of the present disclosure.

FIG. 32 illustrates a balloon catheter device inserted into a sinus seeker instrument secured by overhangs in a slot on the sinus seeker instrument, in accordance with various aspects of the present disclosure.

FIG. 33 illustrates a balloon catheter device inserted into a sinus seeker with the sinus seeker including an interchangeable tip secured by a threaded thumb screw, in accordance with various aspects of the present disclosure.

FIG. 34 illustrates various sinus seeker tip configurations, in accordance with various aspects of the present disclosure.

FIG. 35 illustrates a system including a balloon catheter device, sinus seeker, IGS sensor, and inflation device, in accordance with various aspects of the present disclosure.

### DETAILED DESCRIPTION OF THE INVENTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in a specific order as recited herein. Alternatively, in any aspect(s) disclosed herein, specific acts may be carried out in any order without departing from the principles of the invention, except when a temporal or operational sequence is explicitly recited. Furthermore, specified acts can be carried out concurrently unless explicit claim language recites that they be carried out separately or the plain meaning of the claims would require it. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

### Method of Treatment of a Recurring Airway Stricture or Stenosis in an Airway Body Lumen.

The present invention provides a method of treatment of a recurring airway stricture or stenosis in an airway body lumen. The method can open the body lumen and can prevent, reduce, or minimize re-narrowing and recurring strictures or restenosis of the body lumen. The method can prevent recurrence of the stricture or stenosis, or can cause recurrence to be less frequent and/or can cause recurrence to be less severe as compared to conventional treatments. The treatment can be performed on a variety of animals and humans, such as premature neonates to adult humans. In various aspects, the present invention provides a minimally invasive method for treatment or prevention of recuring strictures of the upper and lower airways.

The body lumen can be any suitable airway body lumen that includes a stricture or stenosis that has recurred at least one time. The body lumen can include a frontal sinus, ethmoid sinus, sphenoid sinus, maxillary sinus, nasal passage, supraglottis, glottis, subglottis, trachea, mainstem bronchus, bronchus intermedius, lobar bronchus, segmental bronchus, or sub-segmental bronchus.

The recurring stricture or stenosis can be caused by any suitable condition. The recurring stricture or stenosis can be idiopathic. The recurring stricture or stenosis can be caused by a surgical treatment. The recurring stricture or stenosis can be caused by a congenital condition, trauma, inflammation, post-intubation tracheal stenosis, post-tracheostomy tracheal restenosis, post-tuberculosis infection, transplant-related restenosis, repeated medication treatments, surgical removal, electrocautery, laser ablation, cryoablation, mechanical debulking, rigid bronchoscopic dilation, stent placement, and/or balloon dilation. The recurring stricture or stenosis can be caused by bronchial smooth muscle cells, IL-4, IL-5, IL-6, IL-13, IL-23, ILC2, mucus plug, goblet cells, fibrosis, cystic fibrosis, and/or one or more mucins. The stricture or stenosis can include CRS stenosis, CRSwNP stenosis, nasal stenosis, severe asthma, comorbidities of CRSwNP, comorbidities of severe asthma stenosis, subglottic stricture and/or stenosis, laryngostenosis, tracheal stenosis, bronchial stenosis, airway anastomotic stenosis, and/or radiation induced airway stenosis. In the treatment of severe asthma, the method can reduce or weaken the smooth muscles in the wall of treated bronchial airways to prevent or reduce recurrence of the severe asthma. In various aspects, the method can include treating CRSwNP and severe asthma in one procedure, including treating at least two of the trachea, mainstem bronchus, bronchus intermedius, lobar bronchus, segmental bronchus, or sub-segmental bronchus. CRSwNP and severe asthma can be comorbidities and treating one can benefit the other. The treatment of CRSwNP can reduce or eliminate recurrence of the severe asthma, and vice versa.

The airway stricture or stenosis can include chronic rhinosinusitis with nasal polyps (CRSwNP). The airway stricture or stenosis can include laryngostenosis and/or subglottic stricture or stenosis. The airway stricture or stenosis can be a stricture or stenosis induced by repeated medication treatments, intubation, tracheostomy, tuberculosis infection, surgical removal, electrocautery, laser ablation, cryoablation, mechanical debulking, rigid bronchoscopic dilation, stent placement, and/or balloon dilation.

The method can include hydrating and/or soaking the coating prior to inflation of the drug-coated balloon. The hydrating and/or soaking can be performed outside the body (e.g., using saline, water, or a combination thereof), during passage to the target site while in the body lumen, at the target site, or a combination thereof. Hydrating and/or soaking that occurs during passage to the target site while in the body lumen, or that occurs at the target site, can include hydrating and/or soaking with flushing composition, natural fluids native to the body lumen that are not externally added, or a combination thereof. In various aspects, for treatment of pulmonary tissue, the method can include pre-soaking the coating prior to insertion to the target site. In various aspects, for treatment of nasal tissue or other non-pulmonary tissue, the method can include pre-soaking the coating prior to insertion to the target site, flushing the target site prior to insertion of the drug-coated balloon to the target site, or a combination thereof.

The method can include flushing the target site with a flushing composition including water and/or saline. The method can include inserting a scope and a balloon catheter into the target site. The flushing can be performed before and/or during the inserting of the scope and balloon catheter into the target site. The balloon catheter can include an elongated balloon. The balloon catheter can also include a coating layer overlying an exterior surface of the balloon. The coating layer can include one or more additives and an initial drug load of a therapeutic agent chosen from paclitaxel, docetaxel, taxol, rapamycin, sirolimus, zotarolimus, everolimus, tacrolimus, an analogue thereof, and a combination thereof. The method can include hydrating and/or soaking the coating in the flushing composition at the target site. The method can include inflating the balloon to an inflation diameter such that the coating contacts an interior of the body lumen at the target site. The method can include deflating the balloon. The method can include withdrawing the scope and the balloon catheter from the target site.

In various aspects of the method, the method is free of damaging, dilating, and/or removing of the stricture or stenosis prior to the insertion of the drug coated balloon catheter. In other aspects that include damaging, dilating, and/or removing of the stricture of stenosis prior to the insertion of the drug coated balloon catheter, the damaging, dilating, and/or removing of the stricture or stenosis can be performed using any suitable method. The damaging, dilating, and/or removing of the stricture or stenosis can include surgical removal, electrocautery, laser ablation, cryoablation, radiofrequency ablation, mechanical debulking, rigid bronchoscopy dilation, knife-cutting, direct vision internal stricturotomy, use of an uncoated balloon to dilate the stricture or stenosis, or a combination thereof. The damaging, dilating, and/or removing of the stricture or stenosis can include inserting a predilation balloon into the body lumen at the target site, inflating the predilation balloon, and removing the predilation balloon prior to inserting the drug coated balloon catheter. In some aspects the predilation balloon catheter has cutting or scoring elements on the balloon that are used to break calcified plaque. In some aspects, the predilation catheter can be shorter and/or of less diameter than the drug-coated balloon treatment catheter. In this scenario, the predilation catheter is positioned such that the center of the balloon body is aligned with the center of the stricture or stenosis. Once inflated, the predilation balloon is deflated and removed and the drug-coated treatment balloon is inserted. The size of the drug coated balloon is chosen such that the balloon diameter and the balloon length is larger than the pre-dilation balloon catheter to ensure the drug coating comes in contact with the entire luminal wall of the predilated stenosis or stricture.

The method includes inserting a balloon catheter through the nasal passage or the mouth and tracking to the recuring airway strictures or stenosis. The inserting the scope and the balloon catheter can include inserting the balloon catheter through a lumen of the scope. The inserting the scope and the balloon catheter can include inserting the balloon catheter and the scope side-by-side. The method can include placing the scope and a proximal edge of the balloon of the balloon catheter at or near the target site. The scope can be any suitable scope for use in the body lumen including the target site, such as an endoscope, rhinolaryngoscope, rhinoscope, bronchoscope, cystoscope, or a combination thereof. The scope can be a rigid scope or a flexible scope. The method can include visualizing positioning of the balloon catheter at the target site with the scope. The method can include visualizing the yielding and dilation of the target site with the scope. The method can include visualizing the inflating with the scope. The method can include the physician/user using the scope to visualize the expansion of the balloon during inflation thereof. The method can include the physician/user using the scope to visualize the expansion of the lumen wall as the inflating balloon presses against the interior of the lumen. The method can include the physician/user using the scope to visualize and ensure complete apposition of the drug coated balloon against the lumen and/or using the scope to visualize and prevent overexpansion of the treated lumen. The method can include the physician/user using the scope to visualize and ensure complete drug coverage of the target site after deflation of the balloon via observation of the drug deposited on the lumen wall.

Various aspects of the method are free of flushing the target site prior to and/or during the insertion of the balloon catheter to the target site. In other aspects that include flushing the target site with a flushing composition prior to and/or during the insertion of the balloon catheter to the target site, the method can include flushing the target site prior to inserting a balloon catheter through the nasal passage or the mouth and tracking to the recuring airway stricture or stenosis. The flushing composition can include water, saline, or a combination thereof. In some aspects, the flushing can be performed before, during, or after the insertion of the balloon catheter to the target site. The hydrating and/or soaking in the flushing composition can be performed for any suitable time period, such as about 0.1 minutes to about 20 minutes, or about 0.1 minutes to about 10 minutes, or about 0.1 minutes to about 5 minutes, or less than or equal to 20 minutes and greater than or equal to 0.1 minutes, 0.2, 0.3, 0.4, 0.5, 0.6, 0.7, 0.8, 0.9, 1, 1.5, 2, 2.5, 3, 3.5, 4, 4.5, 5, 6, 7, 8, 9, 10, 12, 14, 16, 18, or 19 minutes. In aspects of the method that are free of flushing of the target site, the balloon catheter can be flushed or soaked prior to insertion (e.g., soaked and/or hydrated outside the body prior to insertion).

In some aspects, the balloon is inflated until the coating layer contacts walls of the stricture or stenosis and the stricture or stenosis is dilated, with simultaneous transfer of the drug to the stricture or stenosis. In some aspects, the balloon is inflated until the coating layer contacts walls of the stricture or stenosis, the inflation dilates the stricture or stenosis to increase its diameter, such that the contacting with the stricture or stenosis can provide full circumferential transfer of the drug to the wall of the stricture or stenosis. In some aspects, the portion of the balloon that includes the drug (e.g., in aspects including less than 100% of the surface area coated with the drug) can contact the stricture or stenosis uniformly. In other aspects, the contacting of various portions of the surface of the balloon with the stricture or stenosis is non-uniform.

The inflated diameter of the balloon can be any suitable diameter that is achieved during or throughout the inflation period such that a desired ratio of the inflated balloon diameter to the diameter of the body lumen is achieved. The desired ratio can be in the range of 0.5 to 2.0 or greater than or equal to 0.5 and less than or equal to 0.75, 1.0, 1.1, 1.2, 1.3, 1.4, 1.5, 1.6, 1.7, 1.8, 1.9, or 2.0. The inflated diameter of the balloon can correspond to the pressure used to inflate the balloon during the inflation period. In some aspects, the inflated pressure can be the nominal pressure for the balloon, and the inflated diameter of the balloon can be about equal to the nominal diameter of the balloon, or can be less than the nominal diameter of the balloon due to constraint from the stricture or stenosis. In some aspects, the inflated pressure of the balloon during the inflation period can be above or below the nominal pressure and the inflated diameter of the balloon can be, correspondingly, above or below the nominal diameter of the balloon.

The inflating can be performed sufficient to achieve a particular stretch ratio. The stretch ratio is defined herein, unless otherwise indicated, as the ratio of the nominal diameter of the balloon to the diameter of the body lumen in the area being treated by the balloon catheter. The nominal diameter of the balloon is the diameter the balloon achieves in an unrestricted environment at the nominal pressure. The lumen diameter is the average of the diameters of the stricture or stenosis or lesion of the lumen. The inflated balloon diameter can be the actual diameter of the balloon following inflation, which in some aspects can equal to, less than, or greater than the nominal diameter of the balloon. In various aspects, the stretch ratio of the balloon catheter of the present invention makes it more effective for treating airways strictures and stenosis than other catheters. During performance of a method of the present invention, the stretch ratio can be selected to be any suitable ratio that achieves the desired ratio of actual inflated balloon diameter to lumen diameter at the range of pressures used during the method. In various aspects, the stretch ratio of the balloon can be about 1.01 to about 20, 1.31 to 20, or about 1.01 to about 15, or about 1.1 to about 10, 1.2 to 10, 1.3 to 10, 1.31 to 10, 1.4 to 10, 1.5 to 10, 1.6 to 10, 1.7 to 10, 1.8 to 10, 2 to 10, 2.2 to 10, 2.5 to 10, or about 1.0 to about 40 (e.g., less than or equal to 40 and greater than or equal to 1, 1.1, 1.2, 1.4, 1.6, 1.8, 2, 2.5, 3, 4, 5, 6, 7, 8, 9, 10, 12, 14, 16, 18, 20, 22, 24, 26, 28, 30, 32, 34, 36, or 38); such a stretch ratio can result in a desired ratio of inflated balloon diameter to lumen diameter at the pressures used during the inflation period that can be the same, similar to, or different than the stretch ratio, such as about 1.01 to about 20, 1.31 to 40, or about 1.01 to about 15, or about 1.1 to about 10, 1.2 to 10, 1.3 to 10, 1.31 to 10, 1.4 to 10, 1.5 to 10, 1.6 to 10, 1.7 to 10, 1.8 to 10, 2 to 10, 2.2 to 10, 2.5 to 10, or about 1.0 to about 40 (e.g., less than or equal to 40 and greater than or equal to 1, 1.1, 1.2, 1.4, 1.6, 1.8, 2, 2.5, 3, 4, 5, 6, 7, 8, 9, 10, 12, 14, 16, 18, 20, 22, 24, 26, 28, 30, 32, 34, 36, or 38).

The inflating can be performed at least until the target site yields and is dilated. The inflating can be performed such that the ratio of the inflation diameter to a normative body lumen diameter at the target site is about 1.0 to about 20, such as less than or equal to 20 and greater than or equal to 1, 1.1, 1.2, 1.4, 1.6, 1.8, 2, 2.5, 3, 4, 5, 6, 7, 8, 9, 10, 11, 12, 13, 14, 15, 16, 17, 18, or 19. The inflating can be performed such that the ratio of the inflation diameter to a normative body lumen diameter at the target site is about 1.0, 1.1, 1.2, or 1.31 to 10. The inflating can be performed such that the balloon is inflated to a pressure that is equal to or greater than a nominal pressure of the balloon. The inflating can be performed such that a stretch ratio of the inflated diameter of the balloon to a normative body lumen diameter at the target site is about 1.0 to about 20. The inflating can be performed such that a stretch ratio of the inflated diameter of the balloon to a normative body lumen diameter at the target site is about 1.0, 1.1, 1.2, or 1.31 to 10, or about 1.0 to about 20, such as less than or equal to 20 and greater than or equal to 1, 1.1, 1.2, 1.4, 1.6, 1.8, 2, 2.5, 3, 4, 5, 6, 7, 8, 9, 10, 11, 12, 13, 14, 15, 16, 17, 18, or 19. The inflating can be performed such that a ratio of the inflation diameter to a normative body lumen diameter at the target site is about 1.0 to 20, and such that a stretch ratio of the inflated diameter of the balloon to a normative body lumen diameter at the target site of about 1.0 to about 20. The inflating can be performed such that the balloon is inflated to a pressure greater than a nominal pressure of the balloon, and a nominal inflated diameter of the balloon is less than the inflated diameter. Feature (a), or (b), or (c), or (a) and (b), or (a) and (c), or (b) and (c), or (a) and (b) and (c), can be present: (a) the ratio of the inflated balloon diameter to a body lumen diameter at the target site is about 1.0 to about 40 (e.g., less than or equal to 40 and greater than or equal to 1, 1.1, 1.2, 1.4, 1.6, 1.8, 2, 2.5, 3, 4, 5, 6, 7, 8, 9, 10, 12, 14, 16, 18, 20, 22, 24, 26, 28, 30, 32, 34, 36, or 38); or (b) the inflating includes inflating the balloon to a pressure equal to or greater than a nominal pressure of the balloon catheter, and the stretch ratio of a nominal diameter of the balloon catheter to a body lumen diameter at the target site is about 1.0 to about 40 (e.g., less than or equal to 40 and greater than or equal to 1, 1.1, 1.2, 1.4, 1.6, 1.8, 2, 2.5, 3, 4, 5, 6, 7, 8, 9, 10, 12, 14, 16, 18, 20, 22, 24, 26, 28, 30, 32, 34, 36, or 38); or (c) the inflating includes inflating to a pressure greater than the nominal pressure of the balloon catheter, and the nominal diameter of the balloon catheter is less than the inflated balloon diameter; or (d) a combination of (a), (b), and (c). The inflating can include observing pressure within the balloon (e.g., via a pressure gauge external to the patient). The inflating can include inflating the balloon to a first pressure, allowing pressure within the balloon to stabilize while maintaining the first pressure in the balloon for a stabilization period, then resuming increasing pressure in the balloon until the inflation diameter is achieved.

The method can include maintaining the inflation diameter for a suitable period of time. Maintaining the inflation diameter can include keeping the inflation diameter at or above a particular desired diameter. The inflation diameter can increase under a consistent inflation pressure as the stricture or stenosis yields. The method can include maintaining the inflation diameter by keeping the balloon inflated for 1 minute to 7 days, 1 minute to 1 day, 1 minute to 10 minutes, or less than or equal to 7 days and greater than or equal to 1 minute, 2 minutes, 3, 4, 5, 6, 7, 8, 9, 10, 12, 14, 16, 18, 20, 25, 30, 35, 40, 45, 50, 55 minutes, 1 hour, 2, 4, 6, 8, 10, 12, 14, 16, 18, 20, 22 hours, 1 day, 2 days, 3, 4, 5, or 6 days. The method can include maintaining the inflation diameter for a duration sufficient to release the drug into tissue of the target site and/or to prevent or reduce bleeding.

### Balloon Catheter.

The balloon catheter can include an elongated balloon. The balloon catheter can also include a coating layer overlying an exterior surface of the balloon. The coating layer can include one or more additives and an initial drug load of a therapeutic agent chosen from paclitaxel, docetaxel, taxol, rapamycin, sirolimus, zotarolimus, everolimus, tacrolimus, an analogue thereof, and a combination thereof.

The balloon catheter can include a fixed wire balloon catheter, over the wire balloon catheter, rapid exchange balloon catheter, a perfusion balloon catheter, a spaced double balloon, a cutting balloon catheter, a scoring balloon catheter, or an infusion catheter (e.g., a distal perforated drug infusion tube, a perforated balloon, spaced double balloon, porous balloon, or a weeping balloon).

The elongated balloon can have a length of about 20 mm to about 300 mm, or about 20 mm to about 160 mm, or less than or equal to about 300 mm and greater than or equal to about 20 mm, 25, 30, 35, 40, 45, 50, 55, 60, 65, 70, 75, 80, 85, 90, 95, 100, 105, 110, 115, 120, 125, 130, 135, 140, 145, 150, 175, 200, 225, 250, 275, or 290 mm.

The balloon can include a main diameter that is a nominal inflated diameter (e.g., a nominal diameter) of at least 1 mm, or at least 5 mm, or at least 10 mm, or at least 13 mm, or at least 15 mm, or at least 20 mm, or at least 30 mm, or at least 35 mm, or 1-50 mm, 1-40 mm, or 1-30 mm, or less than or equal to 50 mm and greater than or equal to 1 mm, 2, 3, 4, 5, 6, 8, 10, 12, 14, 16, 18, 20, 22, 24, 26, 28, 30, 32, 34, 36, 38, 40, 42, 44, 46, or 48 mm. For a balloon including a neck, the main diameter is the diameter of the larger sections of the balloon. The nominal diameter is determined at the nominal inflation pressure of the balloon, is the diameter normally obtained at the nominal pressure in an unrestricted environment.

In some aspects, the inflated pressure used to determine the main diameter can be any pressure that eliminates any folded or creased areas of the balloon and achieves tautness of the balloon. The inflated pressure used to determine the main diameter can be a pressure such that the inflated balloon has a shape and size that corresponds to the desired shape and size of the balloon during the intended treatment of the body lumen. The inflated pressure used to determine the main diameter can be the nominal pressure of the balloon, such that the nominal diameter of the balloon catheter is equal to the main diameter of the balloon. In some aspects, the inflated diameter of the balloon at the target site during inflation to the nominal pressure is equal to the nominal diameter; however, during actual use, some strictures can prevent achievement of the nominal diameter, or can constrain the inflated balloon to form “dog-bone” shape. The nominal balloon diameter at predetermined pressure (e.g., 2 atm, 3 atm, 6 atm, or 9 atm) can be different for different diameters of balloons for various diseases. For example, nominal diameters of sinus stricture or stenosis balloons can be 6 mm, 8 mm, 10 mm, 12 mm, and 14 mm with balloon lengths of 20 mm, 25 mm, and 30 mm for 6 mm, 8 mm, 10 mm, 12 mm, and 14 mm balloon catheters at 4 atm, 5 atm, 6 atm, 8 atm, or 12 atm inflation. The nominal diameters of the airway stricture or stenosis balloons can be 25 mm, and 30 mm with balloon lengths of 20 mm, 25 mm, 30 mm, 35 mm, 40 mm, 45 mm, 50 mm, 55 mm, and 60 mm balloon catheters at nominal pressure of 2 atm, 3 atm, 4 atm, 6 atm, or 9 atm inflation. Nominal pressure is the pressure required to bring the balloon to its labeled nominal diameter in an unconstrained pressure ramp test. Nominal diameter is the desired diameter that the product is labeled with. All physicians purchase balloons and select balloons for use according to the nominal diameter. The rated burst pressure is the maximum pressure that the balloon can be inflated to and have a very high confidence that it will not burst, a labeling requirement for balloon catheters that is calculated from a statistical analysis of the pressures observed when the balloons burst in an unconstrained pressure ramp test.

Examples of properties of balloon catheter for treating recurring airway strictures or stenosis are shown in Table 1.

TABLE 1
Examples of properties of balloon catheters
for treating the recurring airway strictures.

| | Rated burst | | | |
|---|---|---|---|---|
| Nominal | pressure | Nominal | Rated burst | |
| diameter | diameter | pressure | pressure | Compliance |
| (mm) | (mm) | (atm) | (atm) | (%) |
| 4 | 6 | 3 | 12 | 50 |
| 6 | 8 | 3 | 10 | 30 |
| 8 | 10 | 3 | 9 | 25 |
| 10 | 12 | 3 | 8 | 20 |
| 12 | 15 | 3 | 8 | 17 |
| 15 | 18 | 3 | 7 | 20 |
| 18 | 20 | 3 | 6 | 11 |
| 20 | 23 | 3 | 6 | 15 |
| 23 | 25 | 3 | 6 | 9 |
| 30 | 34 | 2 | 4 | 13 |
| 35 | 40 | 2 | 4 | 14 |
| 40 | 45 | 2 | 4 | 12 |

In some aspects, the balloon catheter properties are equal or similar to those given in Table 2, a single balloon catheter having the ability to achieve a wide range of balloon diameters at relatively high working pressures compared to conventional compliant balloons. Balloons in Table 2 have a unique feature that there are three increasing balloon diameters at three increasing inflation pressure stages. The nominal inflation diameter is the diameter at stage I. The diameter increases about 0.5-4 mm, preferably 0.75-3 mm, most preferably 0.9-2 mm for every stage of pressure increase. For example, a balloon that has a diameter of 15 mm at Pressure I (3 atm) has a diameter of 16.5 mm at pressure II (4.5 atm) and has a diameter of 18 mm at pressure III (7 atm).

TABLE 2
Examples of properties of balloon catheters
for treating airway strictures and stenosis.

| | Diameter at | | |
|---|---|---|---|
| Three | pressure | | |
| inflation | stage I | Diameter at | Diameter at |
| pressure | (mm) | pressure | pressure |
| stages | (Nominal | stage II | stage III |
| (atm) | Diameter) | (mm) | (mm) |
| 3, 6, 10 | 4 | 5 | 6 |
| 3, 6, 10 | 6 | 7 | 8 |
| 3, 5.5, 9 | 8 | 9 | 10 |
| 3, 5, 8 | 10 | 11 | 12 |
| 3, 4.5, 8 | 12 | 13.5 | 15 |
| 3, 4.5, 7 | 15 | 16.5 | 18 |
| 3, 4.5, 6 | 18 | 19 | 20 |
| 3, 4.5, 5.5 | 20 | 21.5 | 23 |
| 3, 4, 5 | 23 | 24 | 25 |
| 2, 3.5, 4.5 | 30 | 32.5 | 35 |
| 2, 3, 4 | 35 | 37.5 | 40 |
| 2, 3, 4 | 40 | 42.5 | 45 |

In various aspects, the balloon catheter can be sufficient such that at a predetermined pressure (e.g., the nominal pressure) the balloon can have any suitable ratio of inflated balloon catheter diameter to a diameter of the body lumen at the location of treatment; for example, at a pressure of about 1 atm (˜101 kPa) to about 30 atm (˜3040 kPa) (e.g., about 1 atm or less, or less than, equal to, or more than about 4 atm, 5, 6, 7, 8, 9, 10, 11, 12, 13, 14, 15, 16, 17, 18, 19, 20, 22, 24, 26, 28, or about 30 atm or more).

The balloon catheter can include the elongated balloon and can be free of other balloons. In other aspects, the balloon catheter can include more than one balloon.

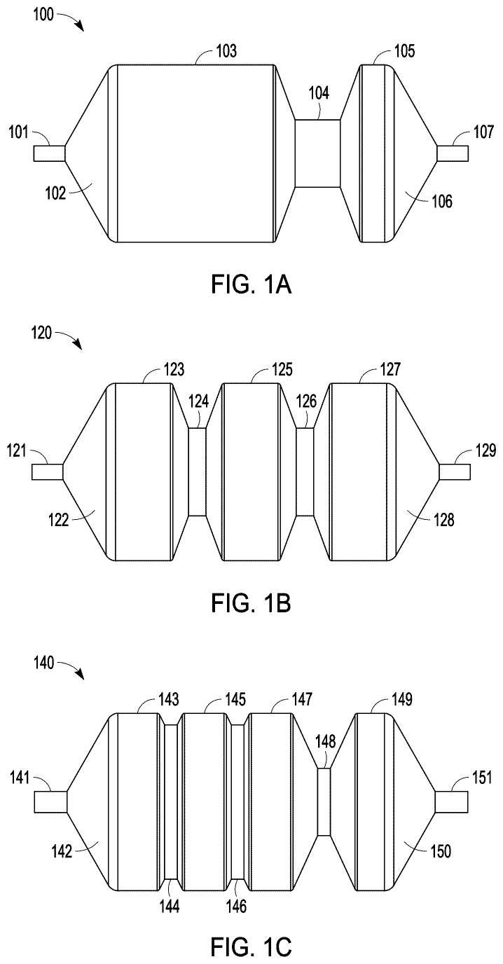

The balloon can be substantially free of neck sections between a proximal and a distal end of the balloon. In other aspects, the balloon can include at least one neck section on the balloon including a smaller diameter than a main diameter of the balloon when the balloon is inflated, the at least one neck section dividing the balloon into at least two main sections each having a diameter. The neck section can include a central narrow portion having the smallest diameter of the neck section, and an adjacent portion that can have a varying diameter and that occurs between the central narrow portion and portions of the balloons having the main diameter. During inflation, the neck section in combination with the at least two main sections can together anchor the balloon at the target site in the body lumen and help to avoid slippage and thereby help to avoid damaging healthy or unintended body lumen. In various aspects, the one or more neck sections can allow the balloon catheter to stay in place during treatment more consistently and effectively to dilate the stricture or stenosis and deliver the drug as compared to other balloon catheters lacking such a neck section or configuration of neck sections. When the diameter of a neck section is referred to herein, it refers to the diameter of the central narrow portion which has the smallest diameter, and not to the tapered sections, unless otherwise indicated. The tapered sections of the balloon can be rigid, flexible (e.g., elastic), or a combination thereof. The diameter of the at least two main sections can be equal to the main diameter of the elongated balloon, or the at least one neck section has a diameter that is about 5% to about 99% of the diameter of at least one of the at least two main sections. The at least one neck section can have a diameter that is independently about 5 mm to about 35 mm, or 1 mm to 40 mm, or less than or equal to 40 mm and greater than or equal to 1 mm, 2, 3, 4, 5, 6, 8, 10, 12, 14, 16, 18, 20, 22, 24, 26, 28, 30, 32, 34, 36, or 38 mm. The one neck section can have any suitable position on the balloon, such as approximately centered with respect to the balloon length, or off-center with respect to the balloon length. The one neck section can be off-center with respect to the length of the balloon and can be at a distal end of the balloon. An aspect of the balloon including one neck section that is off-center with respect to the length of the balloon is illustrated in FIG. 1A.

The diameter of the at least one neck section can be substantially static during inflation of the balloon, such that the diameter of the neck section remains substantially static during inflation of the balloon. The neck section can be a rigid or semi-rigid neck section. The at least one neck section can include a substantially nonelastic (e.g., non-compliant, or minimally compliant) portion of the balloon, a reinforced portion of the balloon, or a combination thereof. The at least one neck section can include an inelastic material around a circumference of the neck section. The inelastic material can include a suture or monofilament or multifilaments of such material, such as nylon, polyamide, an aromatic polyamide, ultra high molecular weight polyethylene (UHMWPE), a polyester, an aromatic polyester, polyethylene terephthalate (PET) or a combination thereof.

The diameter of the at least two main sections can be about 1 mm to about 50 mm, about 1 mm to about 35 mm, about 1 mm to about 30 mm, about 1 mm to about 20 mm, or about 5 mm to about 45 mm, or less than or equal to 50 mm and greater than or equal to 1 mm, 2, 3, 4, 5, 6, 8, 10, 12, 14, 16, 18, 20, 22, 24, 26, 28, 30, 32, 34, 36, 38, 40, 42, 44, 46, or 48 mm.

The at least one neck section can be about 1% to about 50% of the balloon length, or less or equal to about 50% and greater than or equal to 1%, 2, 3, 4, 5, 6, 7, 8, 9, 10, 12, 14, 16, 18, 20, 22, 24, 26, 28, 30, 32, 34, 36, 38, 40, 42, 44, 46, or 48%.

The at least one neck section can be one neck section and the balloon can be free of other neck sections, such that the balloon includes two main sections separated by one neck section. The at least one neck section can be two neck sections and the balloon can be free of other neck sections. The two neck sections can have about the same diameter. One of the two neck sections can have a smaller diameter than the other neck section. The two neck sections can be symmetrically or asymmetrically located with respect to the center of the balloon length. The three main sections can have approximately equal length or can have different lengths. FIG. 1B illustrates an aspect of a balloon catheter having two neck sections with three main sections, wherein the neck sections are symmetrically located about the center of the length of the balloon, and wherein the three main sections of the balloon have about the same length.

The balloon catheter can include three of the main sections separated by the two neck sections. The at least one neck section can be three neck sections, wherein the balloon is free of other neck sections. The three neck sections can be arranged to provide four of the main sections separated by the three neck sections. The three neck sections can be positioned in any suitable way along the length of the balloon. The four main sections formed by the three neck sections can have equal or different lengths. The three neck sections can have equal diameters, or different diameters. In some aspects two of the neck sections have an equal diameter that is smaller than the diameter of the other neck section. FIG. 1C illustrates an aspect of a balloon catheter having three neck sections with four main sections each having an approximately equal length, wherein two of the neck sections have an equal diameter that is smaller than the diameter of the other neck section.

In some aspects, the balloon neck section can be semi-compliant and expand at different rates than the main balloon body. The neck section compliance can be more, less or equal to the compliance of the balloon body. Table 3 illustrates example measurements of a balloon that has neck section that expands more than the balloon body. The expansion rate of the neck diameter can be higher than the expansion rate of the diameter of the main body section in the tested pressure range of 1-5 atm. The expansion rate of the neck diameter can be in the range of 1.1 to 10 times that of the diameter of the main body section (main diameter) in the tested pressure range of 1-5 atm, such as in the range of 2 to 6 times the main diameter. The expansion rate of the neck diameter can be 12.38% per atm. The expansion rate of the body diameter can be 2.4% per atm. The difference of the expansion rates can be 9.98% per atm. Table 4 illustrates example measurements of a balloon that has a neck section that expands less than the balloon body when inflated from 2 atmospheres to 4 atmospheres. The expansion rate of the neck diameter can be less than the expansion rate of the diameter of the main body section in the tested pressure range of 2-4 atm.

TABLE 3
Example measurements of a balloon that has neck
section that expands more than the balloon body.

| Balloon | Neck | Body | | |
|---|---|---|---|---|
| Pressure | Diameter | Diameter | Body | Neck |
| [atm] | [mm] | [mm] | Compliance | Compliance |
| 1 | 21.5 | 39.0 | 2.3% | 12.2% |
| | | | (1-2 atm) | (1-2 atm) |
| 2 | 24.0 | 40.0 | 2.4% | 15.7% |
| | | | (2-3 atm) | (2-3 atm) |
| 3 | 27.5 | 40.5 | 2.6% | 13.4% |
| | | | (3-4 atm) | (3-4 atm) |
| 4 | 31.5 | 41.5 | 2.3% | 8.2% |
| | | | (4-5 atm) | (4-5 atm) |
| 5 | 34.0 | 42.5 | — | — |

TABLE 4
Example measurements of a balloon that has a neck
section that expands less than the balloon body.

| Balloon | Neck | Body | | |
|---|---|---|---|---|
| Pressure | Diameter | Diameter | Body | Neck |
| [atm] | [mm] | [mm] | Compliance | Compliance |
| 2 | 15.28 | 34.06 | 8.7% (2-4 atm) | 0.4% (2-4 atm) |
| 4 | 15.34 | 37.02 | — | — |

The neck section can create a wedge of tissue between the larger diameter sections of the balloon that can hold the balloon in place. The larger sections of the balloon cannot overcome the tissue barriers created at the neck section, thus the balloon with the neck section can prevent, reduce, or minimize balloon migration during inflation. Neck section placement can be designed to facilitate the greatest increase in traction while still maintaining treatment efficacy.

The balloon catheter can include a catheter shaft on a longitudinal end of the balloon, the catheter shaft including an interior lumen for delivery of a gas, liquid, or a combination thereof, to the balloon interior. The balloon catheter can include an atraumatic Coude tip.

The balloon catheter can be a fixed wire balloon catheter. The balloon catheter can be an over-the-wire balloon catheter. The balloon catheter can be a rapid exchange balloon catheter. The outer shaft can be bonded to the proximal balloon neck (e.g., on the proximal end of the balloon, inserted into the body after insertion of the distal end), with the distal end of the tapered wire bonded with a distal neck of balloon, and with the proximal ends of the wire and outer shaft bonded with the hub (e.g., a valve, connector, or adapter) at the proximal end of the balloon catheter. The balloon catheter can be a moveable wire catheter. The outer shaft is bonded to the proximal balloon neck, the distal end of the tapered wire is bonded with the distal neck of balloon, the proximal end of the wire is free to move relative to the hub at the proximal end of the balloon catheter. The balloon catheter can be an over-the wire balloon catheter. The balloon catheter can be a rapid exchanged balloon catheter. The balloon catheter can include a catheter shaft on a longitudinal end of the balloon (e.g., on the proximal end of the balloon, inserted into the body after insertion of the distal end), the catheter shaft including an interior lumen for delivery of air, liquid, or a combination thereof, to the balloon interior. The catheter shaft can include a thermoplastic material that is thermally attached (e.g., attached via heating or melting) to the balloon, such as a high durometer material, such as a material similar or identical to the balloon material, such as polyamides, nylon (e.g., nylon 6,6, or nylon 12), polyether block amide (PEBA), 70 D polyether block amide (PEBA), 72 D polyether block amide (PEBA), 74 D polyether block amide (PEBA), polyethylene terephthalate (PET), polyvinylchloride (PVC), polyester, polyurethane, derivatives thereof, or a combination thereof. In some aspects, the catheter shaft can be a scope (e.g., cystoscope). A high durometer material can help to prevent, reduce, or minimize crushing, and can allow pushability and flexibility. The catheter shaft outer diameter can be sized to allow passage through the working channel of a scope. A fluidic connection between the inner lumen and the inside of the balloon can be included, such as holes in the catheter shaft underneath the balloon attachment point to allow inflation of the balloon by instilling media through the inner lumen.

The catheter shaft at an end that remains outside the body (e.g., proximal end) can include a hub (e.g., a valve, connector, or adapter) that provides a connection to the interior lumen of the catheter shaft. During inflation, the hub (e.g., when closed, or always), can prevent backflow of fluid or air from the balloon. The hub can include any suitable valve, such as a Tuohy Borst adapter. The Tuohy Borst adapter is a compression sealing device that can be placed over the catheter shaft and tightened to provide a liquid/air tight connection to the inner lumen of the catheter shaft. A one-way stopcock can allow control over fluid flow into the balloon and can connects via a standard Luer to an inflation device.

The proximal side of the catheter shaft can include a hub or any other attachment manifold to connect and infuse a liquid, air, or other gas through the catheter to inflate the balloon, allow passage of a guidewire, allow urine to drain, or to make any other suitable connection to the catheter shaft. The connections to the hub or attachment manifold can be made via any suitable one or more connections, such as a single female or male Luer hub, or a Luer manifold with multiple channels. The hub or manifold can be constructed with any appropriate biocompatible material such as polycarbonate, acrylonitrile butadiene styrene, nylon, PEBAX®, silicone, any other polymeric material capable of being molded, or a combination thereof. The hub can be attached to the catheter in any suitable way, such as using adhesive (e.g., cyanoacrylate adhesive, silicone adhesive, epoxy, any other adhesive suitable for the substrates, or a combination thereof) or using chemical bonding. The hub or manifold can be over-molded to the catheter shaft to directly fuse it to the catheter shaft. The catheter shaft can include a strain-relief component at the junction between the catheter shaft and the hub or manifold. The strain-relief component can help to prevent kinking. The strain-relief component can include a polyolefin, PET, FEP, another heat-shrinkable material, or a combination thereof. The strain-relief component can be a heat shrink. The strain-relief component can be a flexible molded material that interfaces with the hub (e.g., the strain-relief component can be attached to the hub).

The balloon catheter can include a catheter tip at a longitudinal end of the balloon, at the distal end which is inserted into the body first. The catheter tip can facilitate passage of the balloon through the body lumen. The tip can be an atraumatic tip that helps prevent damage to the body lumen during insertion therein. The tip can be a Coude atraumatic tip. The atraumatic Coude tip is designed to facilitate passage of the catheter through the bends in body lumen while preventing damage to the walls of the body lumen during tracking. The tip can be a low durometer biocompatible material overmolded onto the catheter shaft or adhesively bonded onto the shaft. For example, the Coude tip can be formed from a PEBAX® or liquid silicone rubber.

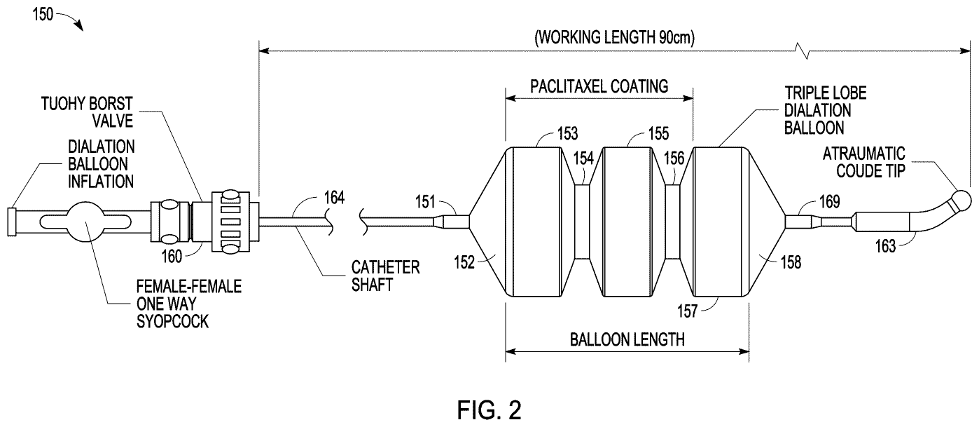

FIG. 2 illustrates an aspect of the balloon catheter including a catheter shaft, catheter tip, and Tuohy Borst adapter/stopcock assembly. All materials can be biocompatible. The balloon is coated with a paclitaxel solution but may be coated with any multitude of other drugs or biologics that could facilitate improvement of BPH symptoms. In FIG. 2, only the proximal two main sections of the balloon are coated with drug.

The balloon catheter can include an inflation device including a pressure gauge or pressure sensor, the inflation device fluidly connected to a catheter shaft connected to the balloon catheter. The gauge or sensor can be used to monitor and/or observe the pressure of the balloon during inflation thereof.

The balloons shown in FIGS. 1A, 1B, 1C, and 2 can be blow molded in a mold that includes the main sections and the neck sections. A tube of balloon material can be inserted into a mold with the desired shape. The balloon material tube may be prestretched. The balloon mold has a shape that corresponds to the balloons shown in FIG. 1A, 1B, 1C, or 2. For a balloon catheter including one neck, this will include a proximal cone, at least one main body section, at least one neck section, at least one more main body section, and the distal cone. The balloon material can be any of polyesters, polyamides, nylon 12, nylon 11, polyamide 12, block copolymers of polyether and polyamide, PEBAX®, polyurethanes, and block copolymers of polyether and polyester. The tube and mold are heated to a temperature above the glass transition temperature of the tube of balloon material and pressurized with gas, air, fluid, or the like resulting in the material of the tube taking the shape of the mold. The formed balloon can then be cooled, trimmed, and is then ready to be attached to the catheter. The balloon catheter shaft can be constructed of polyether-amide block copolymers, polyamides, nylons, polyesters, polyethylene terephthalate, or any other semi-compliant to non-compliant polymer including their blends. The balloon catheter shaft can be constructed using a rigid material such as stainless steel, polycarbonate, titanium, PEEK (polyether ether ketone), or any other rigid biocompatible material. The balloon catheter shaft can include as polyamides, nylon (e.g., nylon 6,6, or nylon 12), a polyether block amide (PEBA) (e.g., 35D PEBA, 55D PEBA, 72D PEBA, or 74D PEBA), polyurethanes, silicones, rubbers, another thermoplastic polymer, or a combination thereof. The catheter shaft can be uniform in composition or can include a combination of materials that are distributed along one or more portions of the catheter shaft. The catheter shaft can be an extruded catheter shaft. In some aspects, the catheter shaft can include an elongated rigid component, such as a rod, mandrel, or wire, aligned longitudinally with the catheter shaft.

After the balloon is attached to a catheter, the balloon can be inflated at a low pressure and a neck section reinforcement can be attached to the neck region. The neck section reinforcement is used to control the expansion of the neck section during balloon expansion. The neck section may be a substantially nonelastic portion of the balloon, a reinforced portion of the balloon, or a combination thereof. The neck section can include an inelastic material circulated around a circumference of the neck section, such as a suture or monofilament or multifilaments of such material, such as steel, stainless steel, nitinol, tungsten, aluminum, copper, silver, gold, platinum, iridium, superalloys contain elements, including nickel (Ni) chromium (Cr), aluminum (Al), titanium (Ti), molybdenum (Mo), tungsten (W), niobium (Nb), tantalum (Ta) and cobalt (Co), nylon, polyamide, aromatic polyamides, ultra high molecular weight polyethylene (UHMWPE), polyesters, aromatic polyesters, polyethylene terephthalate (PET) or a combination thereof. In some aspects, the polymer material is in strand or filament form and is wrapped numerous times around the neck section and then held in place by using two or more spots of glue or adhesive.

The main sections of the balloon may be formed with identical or similar diameters. In some aspects, the diameters of the various main sections may differ from each other by as much as 30%, when measured at nominal balloon diameter. In FIGS. 1A, 1B, 1C, and 2, the main sections of the balloon are shown with equal diameters, that is the diameter of each main section is constant. In practice, at higher pressures, the diameter of the main sections can become slightly bowed out, in that the diameter of the mid part of the main sections may have a slightly larger diameter than the edges of the main sections near the balloon cone and/or near the neck sections.

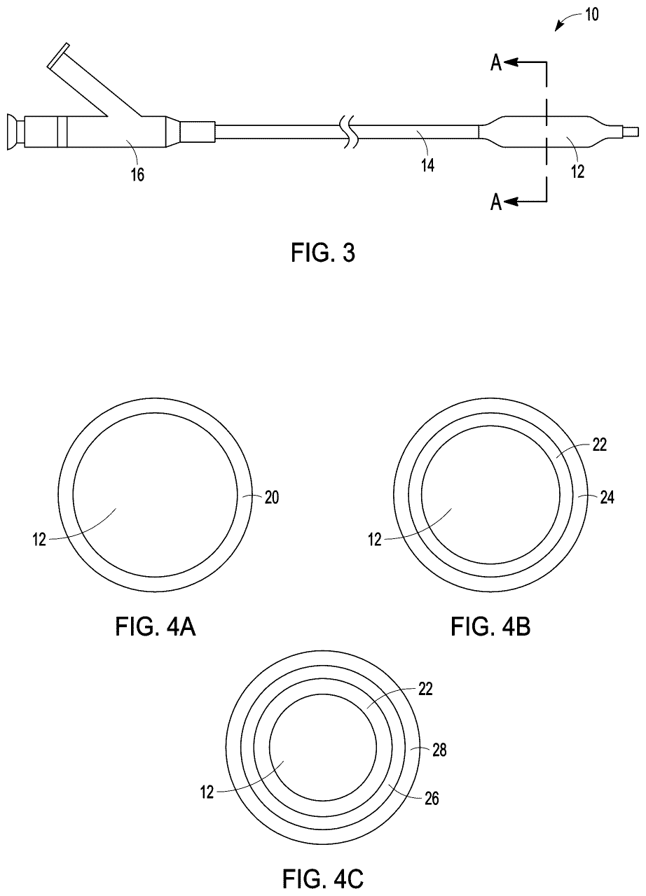

For aspects where the balloon has neck and main sections, as shown in FIGS. 1A, 1B, 1C, and 2, and where the balloon does not have any neck sections, as shown in FIG. 3, after the balloon catheter is assembled, the balloon may be coated with the at least one water-soluble additive and drug as discussed herein. In some aspects where the balloon has multiple main sections, the distal main section may not be coated. The balloon may be coated according to the process discussed herein. If a sheath is used, it may be put over the balloon after the balloon is coated. The catheter can then be packaged, sterilized, and labeled as is known in the art.

The balloon catheter can be any suitable catheter for the desired use, including conventional balloon catheters known to one of ordinary skill in the art. For example, as shown in FIG. 3, balloon catheter 10 can include an expandable, inflatable balloon 12 at a distal end of the catheter 10, a handle assembly 16 at a proximal end of the catheter 10, and an elongate flexible member 14 extending between the proximal and distal ends. Handle assembly 16 can connect to and/or receive one or more suitable medical devices, such as a source of inflation media (e.g., air, saline, or contrast media). Flexible member 14 can be a tube made of suitable biocompatible material and having one or more lumens therein. At least one of the lumens is configured to receive inflation media and pass such media to balloon 12 for its expansion. The balloon catheter can be a rapid exchange or over-the-wire catheter and made of any suitable biocompatible material. The material of balloon 12 can include one or more of polyesters, polyamides, nylon 12, nylon 11, polyamide 12, block copolymers of polyether and polyamide, PEBAX®, polyurethanes, and block copolymers of polyether and polyester.

After the deflation and withdrawal from the target site the balloon catheter can include a residual drug amount that is less than 100 wt % of the initial drug load. After the deflation and withdrawal from the target site the balloon catheter can include a residual drug amount that is about 70 wt % or less of the initial drug load, or that is about 0 wt % to about 99 wt % of the initial drug load, or 1 wt % to 90 wt %, 5 wt % to 90 wt %, 10 wt % to 90 wt %, or less than or equal to 99 wt % and greater than or equal to 0 wt %, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 72, 74, 76, 78, 80, 81, 82, 83, 84, 85, 86, 87, 88, 89, 90, 91, 92, 93, 94, 95, 96, 97, or 98 wt %.

The initial drug load can be from about 1 microgram to about 20 micrograms of the therapeutic agent per square millimeter of the balloon, measured when the balloon is at its nominal inflated diameter, or about 2 to about 6 micrograms of the therapeutic agent per square millimeter of the balloon, or less than or equal to about 20 micrograms per mm² and greater than or equal to about 1 microgram per mm², 2, 3, 4, 5, 6, 7, 8, 9, 10, 11, 12, 13, 14, 15, 16, 17, 18, 19 micrograms per mm².

A ratio by weight of the therapeutic agent in the coating layer to the total weight of the one or more additives in the coating layer can be from about 0.05 to about 20, about 0.5 to about 8, about 2 to about 6, or less than or equal to about 20 and greater than or equal to about 0.05, 0.1, 0.2, 0.3, 0.4, 0.5, 0.6, 0.7, 0.8, 0.9, 1, 1.2, 1.4, 1.6, 1.8, 2, 2.5, 3, 3.5, 4, 4.5, 5, 6, 7, 8, 9, 10, 11, 12, 13, 14, 15, 16, 17, 18, or 19. The drug coating can cover any suitable proportion of the exterior surface of the balloon (e.g., proportion of the surface of the balloon that obtains the main diameter during inflation to the nominal pressure, excluding necks and end-cones), such as about 1% to about 100%, or about 50% to about 100%, to about 80% to about 100%, or about 10% or less, or less than, equal to, or greater than 20%, 30, 40, 50, 60, 70, 75, 80, 85, 90, 95, 96, 97, 98, 99, or about 100% or more.

The balloon catheter can further include a sheath covering the elongated balloon. The method can include removing the sheath before the inflating.

The catheter shaft, the balloon, or a combination thereof, can include single or multiple markings along its length to aid in positioning and alignment with certain anatomical structures. The markings can have any suitable orientation, such as circumferential, or longitudinally along the catheter shaft or balloon. Marks on the catheter shaft or balloon can be used to aid in positioning the balloon at the target site, indicate that the balloon is fully recovered in the sheath, or locate the device within a patient's anatomy. Markings on the catheter shaft can be visualized using a scope, or with the unaided eye, or markings can include radiologically distinguishable components such as radiopaque materials. Markings can be created by thermally bonding polymer to the surface of the catheter shaft having a distinguishable color, via pad print, via laser marking, or via any other method.

FIGS. 4A-4C are cross-sectional views of different aspects of the distal portion of the balloon catheter of FIG. 3, taken along line A-A, showing exemplary coating layers. In some aspects, the balloon can optionally include an adherent layer. For example, as shown in the aspect depicted in FIG. 4A, the balloon 12 is coated with a layer 20 that includes a therapeutic agent and an additive. In some aspects, the layer consists essentially of a therapeutic agent and an additive, e.g., the layer includes only the therapeutic agent and the additive, without any other materially significant components. In some aspects, the device may optionally include an adherent layer. For example, as shown in the aspect depicted in FIG. 4B, the balloon 12 is coated with an adherent layer 22. A layer 24 that includes a therapeutic agent and an additive is overlying the adherent layer. The adherent layer, which is a separate layer underlying the drug coating layer, improves the adherence of the drug coating layer to the exterior surface of the medical device and protects coating integrity. For example, if drug and additive differ in their adherence to the balloon, the adherent layer can prevent differential loss of components and maintain drug-to-additive ratio in the coating during transit to a target site for therapeutic intervention. Furthermore, the adherent layer can function to facilitate rapid release of coating layer components off the device surface upon contact with tissues at the target site. In other aspects, the device can include a top layer. For example, as shown in the aspect depicted in FIG. 4C, the balloon 12 is coated with an adherent layer 22, a layer 26 that includes a therapeutic agent and an additive overlying the adherent layer, and a top layer 28. The top layer can reduce loss of the drug layer before it is brought into contact with target tissues, for example during transit of the balloon 12 to the site of therapeutic intervention or during the first moments of inflation of balloon 12 before coating layer 20 is pressed into direct contact with target tissue.

### Therapeutic Agent.

The balloon catheter can include a coating layer overlaying an exterior surface of the balloon. The coating layer can include one or more additives and initial drug load of a therapeutic agent. The coating layer can be a single layer, or the coating layer can include two or more layers. The coating layer can include one therapeutic agent or more than one therapeutic agent. The therapeutic agent can be in direct contact with the exterior of the balloon, of the balloon can include a coating between the therapeutic agent and the exterior of the balloon, such as a layer including one or more additives.

The therapeutic agent can be a hydrophobic therapeutic agent, an anti-inflammatory drug, and/or an anti-proliferative drug. The therapeutic agent can be of any suitable physical state, such as molecular distribution, crystal forms, or cluster forms. The therapeutic agent can include a lipophilic substantially water insoluble drug, such as paclitaxel, rapamycin, daunorubicin, doxorubicin, lapachone, vitamin D2 and D3, and analogues and derivatives thereof. The therapeutic agent can include paclitaxel, docetaxel, taxol, rapamycin, sirolimus, zotarolimus, everolimus, tacrolimus, mTOR inhibitors (i.e., a class of drugs that inhibit the mechanistic target of rapamycin), an analogue thereof (i.e., an analogue of any member of the list), or a combination thereof. The therapeutic agent can be in any suitable form, such as crystalline, partially crystalline, amorphous, partially amorphous, or a combination thereof. In some aspects, the therapeutic agent can have a particle size of 0.2 microns to 10 microns, or preferably 0.5 microns to 5 microns. In some aspects, the therapeutic agent is at least 75 wt % crystalline or at least 90 wt % crystalline. In another aspect the drug is substantially amorphous with little or no molecular orientation. Techniques to determine if the a drug is crystalline or amorphous include power x-ray diffraction (pXRD), modulated differential scanning calorimetry (mDSC), or confocal raman spectroscopy.

In some aspects, the one or more additives can partially or fully encapsulate the therapeutic agent. For example, the size of particles of the therapeutic agent encapsulated by the one or more additives can be 0.7 microns to 15 microns.

The therapeutic agent can include budesonide, flunisolide, triamcinolone, beclomethasone, fluticasone, mometasone, mometasone furoate, dexamethasone, hydrocortisone, methylprednisolone, prednisone, cortisone, betamethasone, triamcinolone acetonide, or a combination thereof. The therapeutic agent can include a bronchodilator or a vasoconstrictor. The therapeutic agent can include terbutaline, albuterol, ipratropium, pirbuterol, epinephrine, salmeterol, levalbuterol, formoterol, or a combination thereof.

In various aspects, a combination of therapeutic agents can be used. Various drug combinations can have additive effects because of different therapeutic mechanisms. For example, the therapeutic agent can include a combination of paclitaxel and rapamycin, a combination of paclitaxel and active vitamin D, a combination of paclitaxel and lapachone, a combination of rapamycin and active vitamin D, a combination of rapamycin and lapachone, or a combination thereof. In various aspects, due to additive effects of the therapeutic agents, the dose of one or more therapeutic agents can be reduced, reducing or preventing complications caused by a higher dose of one or more of the therapeutic agents.

In various aspects, the therapeutic agent is a polymer-encapsulated drug particle.

### Additive.

The balloon catheter can include a coating layer overlaying an exterior surface of the balloon. The coating layer can include one or more additives and initial drug load of a therapeutic agent.

The one or more additives can include one or more water insoluble additives. The one or more additives can include one or more slightly water insoluble and/or partially water insoluble additives. The one or more additives can include one or more water soluble additives. The one or more additives can include: a) one or more water soluble additives, and b) one or more water insoluble or partially water insoluble additives.

The one or more additives can be chosen from pentaerythritol ethoxylate, pentaerythritol propoxylate, pentaerythritol propoxylate/ethoxylate, N-acetylglucosamine, N-octyl-D-gluconamide, N-nonanoyl-N-methylglycamine, N-octanoyl-N-methyl glutamine, C6-ceramide, dihydro-C6-ceramide, cerabroside, sphingomyelin, galaclocerebrosides, lactocerebrosides, N-acetyl-D-sphingosine, N-hexanoyl-D-sphingosine, N-octonoyl-D-sphingosine, N-lauroyl-D-sphingosine, N-palmitoyl-D-sphingosine, sphingosine, polyethylene glycol (PEG) caprylic/capric diglycerides, PEG-8 caprylic/capric glycerides, PEG caprylate, PEG-8 caprylate, PEG caprate, PEG caproate, glyceryl monocaprylate, glyceryl monocaprate, glyceryl monocaproate, monolaurin, monocaprin, monocaprylin, monomyristin, monopalmitolein, monoolein, creatine, creatinine, agmatine, citrulline, guanidine, sucralose, aspartame, hypoxanthine, theobromine, theophylline, adenine, uracil, uridine, guanine, thymine, thymidine, xanthine, xanthosine, xanthosine monophosphate, caffeine, allantoin, (2-hydroxyethyl) urea, N,N′-bis(hydroxymethyl) urea, glycerol ethoxylate, glycerol propoxylate, trimethylolpropane ethoxylate, pentaerythritol, dipentaerythritol, crown ether, 18-crown-6,15-crown-5,12-crown-4, and combinations thereof. The one or more additives can include pentaerythritol ethoxylate, pentaerythritol propoxylate, or a combination thereof. The one or more additives can include butylated hydroxytoluene (BHT), butylated hydroxyanisole (BHA), a derivative thereof, and/or a combination thereof. The one or more additives can include a polymer. In various aspects, the therapeutic agent can include a polymer-encapsulated drug particle.

The coating layer overlying the exterior of the balloon can include one or more water-soluble additives (e.g., a water-soluble first additive, a water-soluble second additive, and a water-soluble third additive). The water-soluble additive can include a first water soluble additive that is a surfactant such as a PEG sorbitan monolaurate, a PEG sorbitan monooleate, or a combination thereof. The water-soluble additive can include a second water-soluble additive that is a chemical compound with one or more moieties that are hydroxyl, amine, carbonyl, carboxyl, or ester, such as sorbitol, sorbitan, xylitol, gluconolactone, or a combination thereof. The coating layer can include both the first water-soluble additive and the second water-soluble additive. In some aspects, the distal end of the balloon can be free of the therapeutic agent.

In some aspects, the additive is at least one of a surfactant and a chemical compound.

In some aspects the coating with a therapeutic agent and has at least one water soluble component and at least one water insoluble component or partially water-soluble component and the coating releases off the balloon into aqueous media in less than 30 seconds with no agglomerated particles or individual particles greater than 20 μm. In some aspects the coating released has particles or agglomerated particles less than 10 μm, or preferably less than 5 μm particles.

In some aspects, the one or more additives can enhance release of the therapeutic agent off the balloon. The additive can enhance penetration and absorption of the therapeutic agent in tissue. The additive can have a water and ethanol solubility of at least 1 mg/mL and the therapeutic agent can be water-insoluble.

The coating overlying the surface of the balloon can include a therapeutic agent and at least two additives, wherein each of the additives includes a hydrophilic part and a drug affinity part, wherein the drug affinity part is at least one of a hydrophobic part, a part that has an affinity to the therapeutic agent by hydrogen bonding, and a part that has an affinity to the therapeutic agent by van der Waals interactions, and wherein each additive is soluble in polar organic solvent and is soluble in water. In one aspect of this aspect, the polar organic solvent is chosen from methanol, ethanol, isopropanol, acetone, dimethylformide, tetrahydrofuran, methylethyl ketone, dimethylsulfoxide, acetonitrile, ethyl acetate, and chloroform and mixtures of these polar organic solvents with water. In another aspect of this aspect, the balloon further includes a top layer overlying the surface of the layer overlying the exterior surface of the balloon to reduce loss of drug during transit through a body to the target tissue.

The one or more additives can facilitate rapid drug elution and superior permeation of drug into tissues at a stricture or stenosis site. Thus, coatings according to aspects of the present invention provide an enhanced rate and/or extent of absorption of the antiproliferative therapeutic agent in airway strictures or stenosis. In aspects of the present invention, the coated device delivers antiproliferative therapeutic agent to the airway stricture or stenosis during a very brief deployment time of less than 10 minutes, less than 2 minutes, and reduces re-narrowing and reoccurring of the strictures of a nonvascular body lumen.

The additive, such a water-insoluble or slightly or partial water-insoluble additive, can be chosen from cholesteryl acetate, cholesteryl phenylacetate, cholesteryl laurate, cholesteryl palmitate, cholesteryl stearate, cholesteryl n-valerate, cholesteryl benzoate, cholesteryl heptylate, cholesteryl decylate, cholesteryl caproate, cholesteryl oleate, cholesteryl oleyl carbonate, cholesteryl linoleate, cholesteryl pelargonate, cholesteryl erucate, cholesteryl caprylate, 5-α-cholestane, 5α-cholestan-3-one. The water-insoluble or slightly or partial water-insoluble first additive with alkyl fatty group is chosen from alkyl glyceryl ethers, monoglycerides of C8-C12 fatty acids, alkyl alcohol, alkyl ether, alkyl ester, caprylic acid, monocaprilin, capric acid, monocaprin, lauric acid, dodecyl glycerol, butanoic acid, hexanoic acid, octanoic acid, decanoic acid, dodecanoic acid, tetradecanoic acid, hexadecanoic acid, octadecanoic acid, octadecatrienoic acid, eicosanoic acid, cicosenoic acid, cicosatetraenoic acid, cicosapentaenoic acid, docosahexaenoic acid, tocotrienol, butyric acid, caproic acid, caprylic acid, capric acid, lauric acid, myristic acid, palmitic acid, palmitoleic acid, stearic acid, oleic acid, vaccenic acid, linoleic acid, alpha-linolenic acid, gamma-linolenic acid, behenic acid, crucic acid, lignoceric acid, natural or synthetic phospholipids, mono-, di-, or triacylglycerols, cardiolipin, phosphatidylglycerol, phosphatidic acid, phosphatidylcholine, alpha tocopherol, phosphatidylethanolamine, sphingomyelin, phosphatidylserine, phosphatidylinositol, dimyristoylphosphatidylcholine, dioleoylphosphatidylcholine, dipalmitoylphosphatidylcholine, distearoylphosphatidylcholine, phosphatidylethanolamines phosphatidylglycerols, sphingolipids, prostaglandins, gangliosides, neobee, niosomes, and derivatives. and combinations thereof.

The additive, such as a water soluble second additive, can include polyethylene glycol (PEG)-cholesteryl sebacate, polyoxyethanyl α-tocopheryl sebacate, methylated polyethylene glycol cholesterol (mPEG cholesterol), polyethylene glycol cholesterol, PEG amide ester cholesterol, PEG amide ether cholesterol, mPEG amide ester cholesterol, PEG amide ether cholesterol, DSPE-PEG-cholesterol, PEGylated phospholipid, methylated PEGylated phospholipid, PEG caprylic/capric diglycerides, PEG8 caprylic/capric glycerides, PEG caprylate, PEG caprate, PEG caproate, PEG-20 sorbitan monolaurate (Tween-20), PEG-20 sorbitan monopalmitate (Tween-40), PEG-20 sorbitan monostearate (Tween-60), PEG-20 sorbitan monooleate (Tween-80), PEG laurate, PEG oleate, PEG stearate, PEG glyceryl laurate, and PEG-30 glyceryl oleate.

In some aspects, the one or more additives includes a first additive including a water-insoluble or slightly or partial water-insoluble additive including at least one alkyl fatty group or cholesteryl group with a molecular weight of 50 to 750. One or more of the additives, such as a water insoluble additive in the coating, can have a lower melting temperature than its pure form. One or more of the additives, such as a water insoluble additive in the coating, can have a lower crystallinity than that of its pure form. One or more of the additives, such as a water-soluble additive can be more hydrophilic or more water soluble than a water-insoluble or slightly or partial water-insoluble additive in the coating. A water-soluble additive can include a polyethylene glycol (—(CH₂CH₂O)—) unit with a molecular weight in the range of 750 to 100,000, or 750 to 50,000, or 750 to 10,000.

### Method of Treating a Central Airway Obstruction (CAO), Benign Airway Stenosis, Asthma, Chronic Obstructive Pulmonary Disease (COPD), or an Airway Tumor.

Various aspects of the method of treatment of a recuring airway stricture or stenosis in an airway body lumen described herein provide a method of treating a central airway obstruction (CAO), benign airway stenosis, asthma, chronic obstructive pulmonary disease (COPD), or an airway tumor in a human subject. The method can include inserting a drug-coated balloon catheter into a target site in a trachea, mainstem bronchus, bronchus intermedius, lobar bronchus, segmental bronchus, or sub-segmental bronchus. The drug-coated balloon catheter can be any suitable drug-coated balloon catheter described herein. The method can include inflating the balloon of the drug-coated balloon catheter to dilate the airway and deliver a therapeutic agent (i.e., a therapeutic agent in the drug coating of the balloon catheter) to the airway wall. The drug coating on the balloon can include the therapeutic agent and one or more additives. The therapeutic agent can include paclitaxel, sirolimus, or a derivative thereof. The method can include deflating the balloon. The method can include withdrawing the balloon catheter from the target site.

In various aspects, the balloon catheter can be delivered through the working channel of a bronchoscope. In other aspects, the balloon catheter can be delivered side-by-side with a bronchoscope.

The balloon catheter can be an over-the-wire catheter compatible with a guidewire. The guidewire can be any suitable catheter guidewire, such as a guidewire having a 0.035″ diameter.

The balloon of the balloon catheter can have a nominal diameter of 4 mm to 20 mm, or 8 mm to 18 mm, or less than or equal to 20 mm and greater than or equal to 4 mm and less than, equal to, or greater than 5 mm, 6, 7, 8, 9, 10, 11, 12, 13, 14, 15, 16, 17, 18, or 19 mm. The balloon of the balloon catheter can have a length of 30 mm to 65 mm, or less than or equal to 65 mm and greater than or equal to 30 mm and less than, equal to, or greater than 32 mm, 34, 36, 38, 40, 42, 44, 46, 48, 50, 52, 54, 56, 58, 60, 62, or 64 mm.

The method can include inflating the balloon of the balloon catheter to a desired pressure such as a nominal pressure of 2 atm to 12 atm, or 3 atm to 6 atm, or less than or equal to 12 atm and greater than or equal to 2 atm and less than, equal to, or greater than 3 atm, 4, 5, 6, 7, 8, 9, 10, or 11 atm.

The coating layer of the balloon catheter can include the therapeutic agent at any suitable dose density. The coating layer can include the therapeutic agent at a dose density of 1 μg/mm² to 20 μg/mm², or 2 μg/mm² to 5 μg/mm², or less than or equal to 20 μg/mm² and greater than or equal to 1 μg/mm² and less than, equal to, or greater than 2 μg/mm², 3, 4, 5, 6, 7, 8, 9, 10, 11, 12, 13, 14, 15, 16, 17, 18, or 19 μg/mm². The therapeutic agent can be any suitable therapeutic agent described herein. The therapeutic agent can include paclitaxel, sirolimus, or a derivative thereof. The therapeutic agent can include paclitaxel.

The one or more additives can include any suitable one or more additives described herein. The one or more additives can include pentaerythritol ethoxylate (PEE). The weight ratio of the one or more additives to the therapeutic agent in the coating can be from 0.01:1 to 10:1, or 1:1 to 5:1, or 0.1:1 to 0.5:1, or less than or equal to 10:1 and greater than or equal to 0.01:1 and less than, equal to, or greater than 0.05:1, 0.1:1 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.5:1, 3:1, 3.5:1, 4:1, 4.5:1, 5:1, 6:1, 7:1, 8:1, or 9:1.

The balloon catheter can include two radiopaque marker bands indicating the drug-coated working length of the balloon catheter. The balloon catheter can be provided sterile and can be intended for single use.

In various aspects, the method can include soaking the balloon of the balloon catheter in saline or water prior to insertion to hydrate the coating, such as soaking for 0.1 to 5 minutes, or for 1 to 2 minutes, or for less than or equal to 5 minutes and greater than or equal to 0.1 minutes and less than, equal to, or greater than 0.2, 0.3, 0.4, 0.5, 0.6, 0.7, 0.8, 0.9, 1, 1.5, 2, 2.5, 3, 3.5, 4, or 4.5 minutes. In other aspects, the method can be free of soaking the balloon in saline or water prior to insertion of the balloon catheter.

The balloon of the balloon catheter can be kept inflated at a desired pressure at the target site for 1 minute to 10 minutes, or for 2 to 3 minutes, or for less than or equal to 10 minutes and greater than or equal to 1 minute and less than, equal to, or greater than 2 minutes, 3, 4, 5, 6, 7, 8, or 9 minutes.

The method can include monitoring the pressure during inflation and maintaining the balloon at a desired pressure for a predetermined time.

The method can be performed in a subject with post-intubation tracheal stenosis, post-tracheostomy tracheal stenosis, post-transplantation stenosis, or post-COVID airway stenosis.

The method can be performed in a subject with a Myer-Cotton grade 2, 3, or 4 airway stenosis. The method can result in a reduction in the Myer-Cotton grade of the stenosis by at least one grade at 1 month post-procedure (e.g., reduction by 1 grade, 2, or 3 grades).

The method can result in a freedom from symptom-driven target lesion reintervention of at least 80% at 6 months post-procedure, or 86% to about 90% at 6 months post-procedure, or less than or equal to 100% and greater than or equal to 80% and less than, equal to, or greater than 81, 82, 83, 84, 85, 86, 87, 88, 89, 90, 91, 92, 93, 94, 95, 96, 97, 98, or 99%.

The method can result in an improvement in FEV1 of at least 100 mL in about 50% to about 70% of patients at 12 months post-procedure, or in 60% to 70%, or less than or equal to 70% and greater than or equal to 50% and less than, equal to, or greater than 51, 52, 53, 54, 55, 56, 57, 58, 59, 60, 61, 62, 63, 64, 65, 66, 67, 68, or 69%.

The method can result in an improvement in mMRC dyspnea score of at least 1 grade in about 75% to about 90% of patients at 12 months post-procedure, or 80% to 90%, or less than or equal to 90% and greater than or equal to 75% and less than, equal to, or greater than 76%, 77, 78, 79, 80, 81, 82, 83, 84, 85, 86, 87, 88, or 89%.

The method can result in an improvement in Peak Expiratory Flow of greater than 18.8 L/min in about 85% to about 95% of patients at 12 months post-procedure, or 90% to 95%, or less than or equal to 95% and greater than or equal to 85% and less than, equal to, or greater than 86%, 87, 88, 89, 90, 91, 92, 93, or 94%.

Method of Treating Chronic Rhinosinusitis (CRS) or Chronic Rhinosinusitis with Nasal Polyps (CRSwNP).

Various aspects of the method of treatment of a recurring airway stricture or stenosis in an airway body lumen described herein can include a method of treating chronic rhinosinusitis (CRS) or chronic rhinosinusitis with nasal polyps (CRSwNP) in a human subject. The method can include identifying a target lesion in a paranasal sinus or sinus drainage pathway. The method can optionally include performing surgical dissection, irrigation, or balloon dilation with an uncoated balloon as needed; in other aspects, surgical dissection, irrigation, or balloon dilation is not needed or has been previously performed prior to the onset of the method at the lesion location. The method can include inserting a drug-coated balloon catheter into the target lesion. The drug-coated balloon catheter can be any suitable drug-coated balloon catheter described herein. The method can include inflating the balloon to dilate the lesion and deliver a therapeutic agent (i.e., a therapeutic agent in the drug coating of the balloon catheter) to the tissue. The drug coating on the balloon can include the therapeutic agent and one or more additives. The therapeutic agent can include paclitaxel, sirolimus, or a derivative thereof. The method can include deflating the balloon. The method can also include withdrawing the balloon catheter.

The balloon of the balloon catheter can be selected to have a nominal inflated diameter that is 1 mm to 10 mm larger than the prepared lesion, or 2 mm to 3 mm larger, or less than or equal to 10 mm and greater than or equal to 1 mm and less than, equal to, or greater than 2 mm, 3, 4, 5, 6, 7, 8, or 9 mm.

In various aspects, the method can include soaking the balloon of the balloon catheter in saline or water prior to insertion to hydrate the coating, such as soaking for 0.1 to 5 minutes, or for 1 to 2 minutes, or for less than or equal to 5 minutes and greater than or equal to 0.1 minutes and less than, equal to, or greater than 0.2, 0.3, 0.4, 0.5, 0.6, 0.7, 0.8, 0.9, 1, 1.5, 2, 2.5, 3, 3.5, 4, or 4.5 minutes. In other aspects, the method can be free of soaking the balloon in saline or water prior to insertion of the balloon catheter.

The balloon of the balloon catheter can be kept inflated at a desired pressure at the target site for 1 minute to 10 minutes, or for 2 to 3 minutes, or for less than or equal to 10 minutes and greater than or equal to 1 minute and less than, equal to, or greater than 2 minutes, 3, 4, 5, 6, 7, 8, or 9 minutes.

The balloon catheter can be a rapid exchange catheter with a semi-compliant sinus dilation balloon.

The balloon of the balloon catheter can have a labeled diameter of 7 mm to 20 mm, or 10 mm to 20 mm, or less than or equal to 20 mm and greater than or equal to 7 mm and less than, equal to, or greater than 8 mm, 9, 10, 11, 12, 13, 14, 15, 16, 17, 18, or 19 mm. The balloon of the balloon catheter can have a length of 20 mm to 35 mm or less than or equal to 35 mm and greater than or equal to 20 mm and less than, equal to, or greater than 21 mm, 22, 23, 24, 25, 26, 27, 28, 29, 30, 31, 32, 33, or 34 mm.

The coating layer of the balloon catheter can include the therapeutic agent at any suitable dose density. The coating layer can include the therapeutic agent at a dose density of 1 μg/mm² to 20 μg/mm², or 2 μg/mm² to 5 μg/mm², or less than or equal to 20 μg/mm² and greater than or equal to 1 μg/mm² and less than, equal to, or greater than 2 μg/mm², 3, 4, 5, 6, 7, 8, 9, 10, 11, 12, 13, 14, 15, 16, 17, 18, or 19 μg/mm². The therapeutic agent can be any suitable therapeutic agent described herein. The therapeutic agent can include paclitaxel, sirolimus, or a derivative thereof. The therapeutic agent can include paclitaxel. The coating layer of the balloon of the balloon catheter can be coated on the cylindrical area between the balloon cones, and optionally on the distal cone. In some aspects, the distal cone includes the coating. In other aspects, the distal cone is free of the coating.

The one or more additives can include any suitable one or more additives described herein. The one or more additives can include pentaerythritol ethoxylate (PEE). The weight ratio of the one or more additives to the therapeutic agent in the coating can be from 0.01:1 to 10:1, or 1:1 to 5:1, or 0.1:1 to 0.5:1, or less than or equal to 10:1 and greater than or equal to 0.01:1 and less than, equal to, or greater than 0.05:1, 0.1:1 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.5:1, 3:1, 3.5:1, 4:1, 4.5:1, 5:1, 6:1, 7:1, 8:1, or 9:1.

The method can result in a freedom from target lesion reintervention due to recurrence of CRS or CRSwNP of at least 90% at 6 months post-procedure, or less than or equal to 100% and greater than or equal to 90% and less than, equal to, or greater than 91%, 92, 93, 94, 95, 96, 97, 98, or 99%.

The method can result in a mean change from baseline in SNOT-22 score of at least 8.9 points at 12 months post-procedure, or at least 27 points, or less than or equal to 35 points and greater than or equal to 8.9 points and less than, equal to, or greater than 9 points, 10, 11, 12, 13, 14, 15, 16, 17, 18, 19, 20, 21, 22, 23, 24, 25, 26, 27, 28, 29, 30, 31, 32, 33, or 34 points.

The method can result in a mean change from baseline in Lund-Mackay CT score of at least 5 points at 12 months post-procedure, or 5 to 20 points, or less than or equal to 20 points and greater than or equal to 5 points and less than, equal to, or greater than 6 points, 7, 8, 9, 10, 11, 12, 13, 14, 15, 16, 17, 18, or 19 points.

The method can result in the therapeutic agent being measurable in blood of fewer than 25% of patients at any time point following treatment, or greater than, equal to, or less than 24%, 23, 22, 21, 20, 19, 18, 17, 16, 15, 14, 13, 12, 11, 10, 9, 8, 7, 6, 5, 4, 3, 2, or 1%. The method can result in the therapeutic agent not being measurable in blood of any patients by day 7 following treatment.

### Method of Reshaping and Flattening Out Residual Bony Partitions in a Surgically Resected Ethmoid Sinus Cavity.

Various aspects of the present invention provide a method of reshaping and flattening out residual bony partitions in a surgically resected ethmoid sinus cavity. The method can include performing surgical ethmoidectomy to create a resected ethmoid cavity. The method can include inserting a balloon catheter into the resected ethmoid cavity. The method can include inflating the balloon to reshape and flatten residual bony partitions. The method can include deflating and removing the balloon. In some embodiments, the method of reshaping and flattening out residual bony portions in a surgically resected ethmoid sinus cavity is an embodiment of the method described herein for treating chronic rhinosinusitis (CRS) or chronic rhinosinusitis with nasal polyps (CRSwNP). In other embodiments, the method of reshaping and flattening out residual bony portions in a surgically resected ethmoid sinus cavity is a stand-alone method that does not require treating chronic rhinosinusitis (CRS) or chronic rhinosinusitis with nasal polyps (CRSwNP).

The balloon can be a drug-coated balloon such as any suitable drug-coated balloon catheter described herein. The drug coating can include a therapeutic agent and one or more additives including pentaerythritol ethoxylate (PEE). The therapeutic agent can include paclitaxel, sirolimus, or a derivative thereof. The therapeutic agent can include paclitaxel.

The balloon can be inserted and inflated at least 1 day after ethmoidectomy. The balloon can be inserted and inflated 1 to 30 days after ethmoidectomy, such as less than or equal to 30 days and greater than or equal to 1 days and less than, equal to, or greater than 2 days, 3, 4, 5, 6, 7, 8, 10, 12, 14, 16, 18, 20, 22, 24, 26, or 28 days.

### A Method of Treating a Eustachian Tube Disorder.

Various aspects of the present invention provide a method of treating a eustachian tube disorder in a human subject. The method can include inserting a balloon catheter into the eustachian tube. The drug-coated balloon catheter can be any suitable drug-coated balloon catheter described herein. The method can include inflating the balloon to dilate the eustachian tube and deliver a therapeutic agent to the tissue (i.e., a therapeutic agent in the drug coating of the balloon catheter). The drug coating on the balloon can include the therapeutic agent and one or more additives. The therapeutic agent can include paclitaxel, sirolimus, or a derivative thereof. The method can include deflating the balloon. The method can include withdrawing the balloon catheter.

The balloon of the balloon catheter can have a nominal inflated diameter of 1 mm to 10 mm, or 2 mm to 3 mm larger, or less than or equal to 10 mm and greater than or equal to 1 mm and less than, equal to, or greater than 2 mm, 3, 4, 5, 6, 7, 8, or 9 mm. The balloon of the balloon catheter can have a length of 10 mm to 30 mm, or less than or equal to 30 mm and greater than or equal to 10 mm and less than, equal to, or greater than 11 mm, 12, 13, 14, 15, 16, 17, 18, 19, 20, 21, 22, 23, 24, 25, 26, 27, 28, or 29 mm.

The balloon of the balloon catheter can be kept inflated at a desired pressure at the target site for 1 minute to 10 minutes, or for 2 to 3 minutes, or for less than or equal to 10 minutes and greater than or equal to 1 minute and less than, equal to, or greater than 2 minutes, 3, 4, 5, 6, 7, 8, or 9 minutes.

The method can result in improved eustachian tube function as measured by tympanometry or patient-reported outcomes.

### A Device for Treating Nasal Sinuses or Paranasal Sinus Drainage Pathways.

Various aspects of the present invention provide a device for treating nasal sinuses or paranasal sinus drainage pathways. The device can be a balloon catheter device. The device can include a tubular balloon catheter with an integrated inflation lumen. The tubular balloon catheter can include a catheter shaft on which the balloon is attached, with the inflation lumen running through the catheter shaft. The device can include a hollow region under the balloon. The device can include an access port in the distal portion of the device (i.e., in the catheter shaft) but proximal to the balloon, through which a positioning instrument may be inserted.

In a pre-inflation configuration, the balloon can be pleated. The balloon can be crimped. The balloon is pleated and crimped.

The device can have a distal tip. The distal tip can be an open tip. The distal tip can be a closed tip.

The device can further include one or more sensors fused or embedded into the distal portion of the device for use with an image guidance system (IGS).

The balloon can include a coating layer that includes a therapeutic agent. The coating layer of the balloon can include the therapeutic agent at any suitable dose density. The coating layer can include the therapeutic agent at a dose density of 1 μg/mm² to 20 μg/mm², or 2 μg/mm² to 5 μg/mm², or less than or equal to 20 μg/mm² and greater than or equal to 1 μg/mm² and less than, equal to, or greater than 2 μg/mm², 3, 4, 5, 6, 7, 8, 9, 10, 11, 12, 13, 14, 15, 16, 17, 18, or 19 μg/mm². The therapeutic agent can be any suitable therapeutic agent described herein. The therapeutic agent can include paclitaxel, sirolimus, or a derivative thereof. The therapeutic agent can include paclitaxel.

The access port can be visually marked or color-contrasted for identification. The device can include markings on the balloon or on a catheter shaft of the balloon to indicate the relative position of the device within the anatomy.

The balloon can have a diameter when inflated at a nominal pressure of 5 mm to 20 mm, or less than or equal to 20 mm and greater than or equal to 5 mm and less than, equal to, or greater than 6 mm, 7, 8, 9, 10, 11, 12, 13, 14, 15, 16, 17, 18, or 19 mm. The balloon can have a body length of 6 mm to 35 mm, or less than or equal to 35 mm and greater than or equal to 6 mm and less than, equal to, or greater than 7 mm, 8, 10, 12, 14, 16, 18, 20, 22, 24, 26, 28, 30, 32, or 34 mm.

In various aspects, the device can include a dual lumen design that includes an integrated inflation lumen and a stiffening member in the catheter shaft. The stiffening member can extend from a fluid input port to a proximal edge of the instrument insertion port.

### System.

Various aspects of the present invention provide a system for performing a method of the present invention. The system can be a system for performing the method of treating chronic rhinosinusitis (CRS) or chronic rhinosinusitis with nasal polyps (CRSwNP) in a human subject described herein. The system can include a balloon dilation device, such as the device for treating nasal sinuses or paranasal sinus drainage pathways described herein. The device can be a balloon catheter device. The device can include a tubular balloon catheter with an integrated inflation lumen. The tubular balloon catheter can include a catheter shaft on which the balloon is attached, with the inflation lumen running through the catheter shaft. The device can include a hollow region under the balloon. The device can include an access port in the distal portion of the device (i.e., in the catheter shaft) but proximal to the balloon, through which a positioning instrument may be inserted. The system can include a positioning instrument configured to be inserted into the balloon device through the access port in the distal portion of the device but proximal to the balloon.

In various aspects, the system can further include an inflation device for inflating the balloon. In various aspects, the system can further include a navigation system for navigating the balloon to a target location.

The positioning instrument can include a sinus seeker with a preconfigured tip shape. The sinus seeker can be integrated with the balloon catheter via a channel with features to maintain the catheter shaft in place during use, such as detent features in a handle of the sinus seeker. The sinus seeker can include an IGS sensor attached to a handle thereof. The positioning instrument can include interchangeable tips secured to a handle of the positioning instrument via a suitable attachment means, such as by a set screw or threaded attachment.

The balloon can include a coating thereon that includes a therapeutic agent. The coating layer of the balloon can include the therapeutic agent at any suitable dose density. The coating layer can include the therapeutic agent at a dose density of 1 μg/mm² to 20 μg/mm², or 2 μg/mm² to 5 μg/mm², or less than or equal to 20 μg/mm² and greater than or equal to 1 μg/mm² and less than, equal to, or greater than 2 μg/mm², 3, 4, 5, 6, 7, 8, 9, 10, 11, 12, 13, 14, 15, 16, 17, 18, or 19 μg/mm². The therapeutic agent can be any suitable therapeutic agent described herein. The therapeutic agent can include paclitaxel, sirolimus, or a derivative thereof. The therapeutic agent can include paclitaxel.

The one or more additives can include any suitable one or more additives described herein. The one or more additives can include pentaerythritol ethoxylate (PEE). The weight ratio of the one or more additives to the therapeutic agent in the coating can be from 0.01:1 to 10:1, or 1:1 to 5:1, or 0.1:1 to 0.5:1 or less than or equal to 10:1 and greater than or equal to 00.1:1 and less than, equal to, or greater than 0.05:1, 0.1:1, 0.2:1, 0.3:1, 0.4:1, 0.5:1, 0.6:1, 0.7:1, 0.8:1, 0.9:1, 1:1, 1.1:1, 1.2:1, 1.3:1, 1.4:1, 1.5:1, 1.6:1, 1.7:1, 1.8:1, 1.9:1, 2:1, 2.5:1, 3:1, 3.5:1, 4:1, 4.5:1, 5:1, 6:1, 7:1, 8:1, 9:1.

### Method of Positioning a Balloon Catheter in a Nasal Cavity, Paranasal Sinus, or Sinus Drainage Pathway.

Various aspects of the present invention provide a method of positioning a balloon catheter in a nasal cavity, paranasal sinus, or sinus drainage pathway. The method can be a method of using the system described herein for performing a method of the present invention, such as the system for performing the method of treating chronic rhinosinusitis (CRS) or chronic rhinosinusitis with nasal polyps (CRSwNP) in a human subject described herein. The method can include inserting a positioning instrument into a port in the distal end of the balloon catheter, proximal to the balloon. The method can include positioning the balloon catheter and positioning instrument into the nasal cavity and directing the balloon catheter to the target location.

The method can be performed under endoscopic visualization. The method can further include confirming the location using an image guidance system (IGS), wherein an IGS sensor is located in the distal part of the balloon catheter or connected to the positioning instrument.

### EXAMPLES

### Part I. Studies Using Sheep.

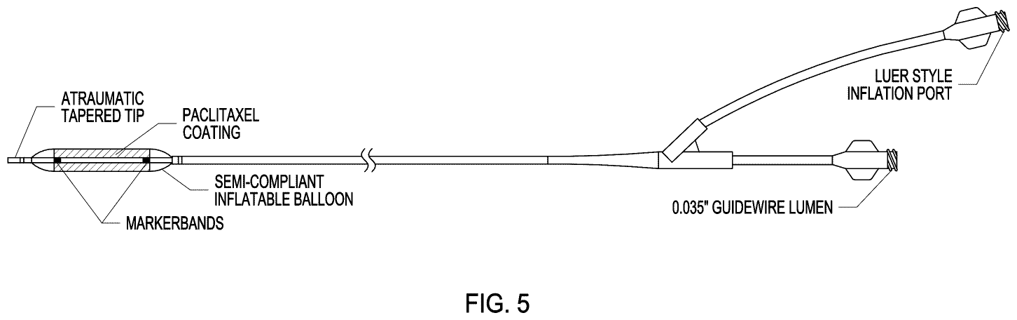

The device used was a 0.035″ guidewire compatible over-the-wire catheter with a tapered atraumatic tip. The distal end of the catheter had a semi-compliant inflatable balloon coated with a proprietary coating containing the drug paclitaxel and carriers that facilitates the drug's transfer to the airway wall upon inflation. The drug coating covers only the balloon body. The device had two radiopaque marker bands that indicated the drug coated working length of the balloon under fluoroscopy. The device was provided sterile and is intended for single use only. FIG. 5 illustrates the device, and Table 5 summarizes the specifications of the device.

TABLE 5
DCB specifications.

| | DRUG | NOMINAL | NOMINAL | MIN | | |
|---|---|---|---|---|---|---|
| | DOSE | BALLOON | BALLOON | RATED | | |
| | (Paclitaxel) | OD | LENGTH | BURST | Shaft | Bronchoscope |
| BALLOON | (mg) | [mm] | [mm] | (ATM) | Length | Compatibility |
| 8 × 30 | 2.6 | 8.0 @ 6 | 30.0 | 12 | 75 cm | ≥2.8 mm |
| | | ATM | | | | Working |
| 10 × 30 | 3.3 | 10.0 @ 6 | 30.0 | 10 | | Channel |
| | | ATM | | | | |
| 12 × 30 | 4.0 | 12.0 @ 6 | 30.0 | 8 | | |
| | | ATM | | | | |
| 14 × 30 | 4.6 | 14.0 @ 6 | 30.0 | 8 | | |
| | | ATM | | | | |
| 12 × 65 | 8.6 | 12.0 @ 3 | 65.0 | 7 | 240 cm | ≤60 cm |
| | | ATM | | | | Total |
| 15 × 65 | 10.7 | 15.0 @ 3 | 65.0 | 6 | | Length |
| | | ATM | | | | |
| 18 × 65 | 12.9 | 18.0 @ 3 | 65.0 | 5 | | |
| | | ATM | | | | |

The amount of drug applied to the surface of the largest DCB (12.9 mg for the 18×65 DCB) is anticipated to be approximately 30 times lower than a typical chemotherapeutic systemic injectable dose of 333 mg paclitaxel/adult man (assumes dosing of 175 mg paclitaxel/m₂ and 1.9 m² surface area for a man). The dose density for each catheter is 3.5 μg paclitaxel/mm² of balloon surface. Table 6 details the amount of drug delivered by balloon size.

TABLE 6
Nominal Paclitaxel Dose per Balloon (mg).

| BALLOON | Drug Dose (mg Paclitaxel) |
|---|---|
| 8 × 30 | 2.6 |
| 10 × 30 | 3.3 |
| 12 × 30 | 4.0 |
| 14 × 30 | 4.6 |
| 12 × 65 | 8.6 |
| 15 × 65 | 10.7 |
| 18 × 65 | 12.9 |

Two preclinical studies are described in Examples 1 and 2. A total of 10 sheep have been treated with the device and to analyze product design feasibility and procedural safety in these anatomies.

Acute Preclinical Study #1 was an acute study conducted on one live sheep to determine acute drug transfer and further refine bronchoscope access techniques, device compatibility, and tissue collection techniques.

Chronic Preclinical Study #2 was a chronic (28 day) study with nine sheep to assess gross pathology of the treatment sites, pharmacokinetics of plasma, treatment sites, and organs, and assess overall animal health during the 28 day in-life phase of the study. Animal safety, device performance, and procedural information was gathered such as overall health of the animals, weight gain over time, treatment site, plasma, downstream, and organ paclitaxel pharmacokinetics, gross pathology, and histology. Results and conclusions from these studies are summarized in Examples 1 and 2 herein. In Examples 1 and 2, the drug-coated balloons were soaked prior to treatment to activate the drug coating.

Examples 3 and 4 describe acute and chronic studies on the nasal tissue with 5 live sheep. In Examples 3 and 4, the nasal treatment sites were flushed with a solution that was saline or water, the balloon catheter was inserted to the nasal treatment site, the balloon catheter was soaked at the site to hydrate and activate the drug coating at the site of treatment, prior to inflation. The drug coated catheters were inflated at the target site for 3-5 minutes.

### Example 1. Acute Preclinical Study #1

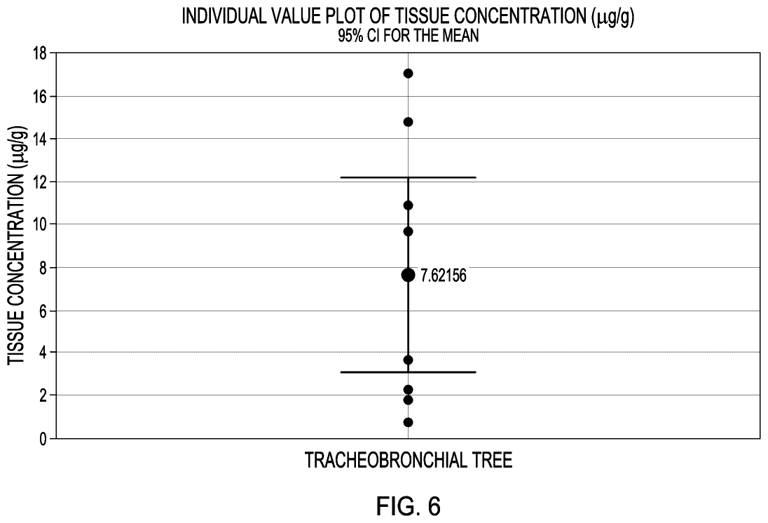

Acute drug transfer was measured in Acute Preclinical Study #1. FIG. 6 illustrates 1-hour paclitaxel drug concentration in the treated tissues. FIG. 6 demonstrates that paclitaxel can be successfully delivered to the trachea and bronchi on a drug coated balloon.

### Example 2. Chronic Preclinical Study #2

### Overall Animal Health (Moribundity)

All assessments suggested that animals remained in good general health throughout the duration of the study. There were no deaths or major adverse events that affected animal health or welfare.

### Treatment Site (Tracheobronchial) Paclitaxel Pharmacokinetics

Animals were terminated at 7, 14, and 28 days. At the time of termination, one animal from each timepoint was used for pharmacokinetic data and each treatment site in the tracheobronchial tree was excised and assayed via HPLC-MS for paclitaxel.

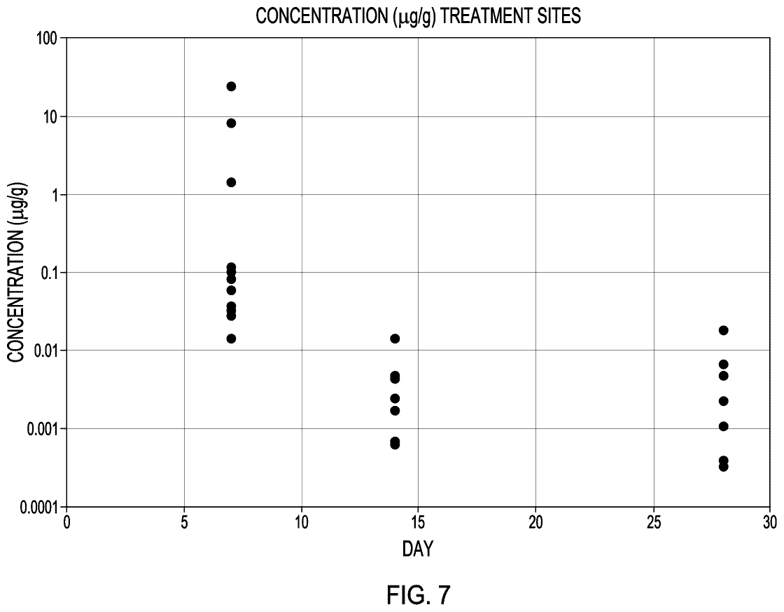

FIG. 7 indicates paclitaxel was delivered to the tissue at the time of treatment and then the paclitaxel concentration decreases with increased time passed. In Examples 1 and 2, the drug coated balloon catheters were soaked and/or hydrated in water or saline solutions for 90 seconds outside the body before inserting the balloon catheter to the target sites in tracheal and bronchial airways. The target sites in tracheal and bronchial airways were not flushed. At 28 days there are low levels of paclitaxel in the tissue of the treatment site. At the treated tissue, the Tₘₐₓ was at one hour, Cₘₐₓ was 6.2 ug/g, and AUC was 52.3 ug/day/g. At the day 28, the average tissue drug level was 3.4 ng/g. The data shown in FIG. 7 is shown in Table 7.

TABLE 7

| | | | | | | Pre- | | | | | | Total | |
|---|---|---|---|---|---|---|---|---|---|---|---|---|---|
| Treat- | Time- | | | | Label | Soak | Inflation | Total | Percent of | Tissue | Con- | Pac- | Con- |
| ment | point | | Part of | Balloon | Claim | Time | Time | PTX | Label | Weights | centration | litaxel | centration |
| Site | (d) | Tissue | Lung | Size | (ug) | (s) | (min) | (ug) | Claim | (g) | (ng/g) | (ng) | (μg/g) |
| Lungs | 7 | Balloon 1 | Bronchi | 10 × 30 | 3299 | 90 | 3 | 2029.9 | 62% | 0.46938 | 27.9 | 13.1 | 0.0279 |
| Lungs | 7 | Balloon 10 | Trachea | 18 × 65 | 12865 | 90 | 2 | 7528.6 | 59% | 12.39149 | 30 | 372 | 0.03 |
| Lungs | 7 | Balloon 11 | Trachea | 18 × 65 | 12865 | 90 | 2 | 5949.6 | 46% | 12.79954 | 30.4 | 389 | 0.0304 |
| Lungs | 7 | Balloon | Trachea | 18 × 65 | 12865 | 90 | 2 | 5308.5 | 41% | 8.82269 | 35.4 | 312 | 0.0354 |
| | | 12a | | | | | | | | | | | |
| Lungs | 7 | Balloon 2 | Bronchi | 12 × 30 | 3958 | 90 | 3 | 2775 | 70% | 0.71741 | 99.2 | 71.2 | 0.0992 |
| Lungs | 7 | Balloon 3 | Bronchi | 14 × 30 | 4618 | 90 | 3 | 3207.3 | 69% | 1.60067 | 23600 | 37800 | 23.6 |
| Lungs | 7 | Balloon 4 | Bronchi | 15 × 55 | 9072 | 90 | 3 | 5073.4 | 56% | 4.7735 | 1420 | 6780 | 1.42 |
| Lungs | 7 | Balloon 5 | Bronchi | 12 × 30 | 3958 | 90 | 3 | 2700.2 | 68% | 2.29622 | 64.1 | 147 | 0.0641 |
| Lungs | 7 | Balloon 6 | Bronchi | 12 × 30 | 3958 | 90 | 3 | 1798 | 45% | 1.85775 | 8060 | 15000 | 8.06 |
| Lungs | 7 | Balloon 7 | Bronchi | 14 × 30 | 4618 | 90 | 3 | 2831.8 | 61% | 1.09789 | 81.7 | 89.7 | 0.0817 |
| Lungs | 7 | Balloon 8 | Bronchi | 10 × 30 | 3299 | 90 | 3 | 1014 | 31% | 1.91407 | 23600 | 45200 | 23.6 |
| Lungs | 7 | Balloon 9 | Bronchi | 18 × 65 | 12865 | 90 | 3 | 6884.7 | 54% | 4.23841 | 113 | 479 | 0.113 |
| Lungs | 14 | Balloon 1 | Bronchi | 10 × 30 | 3299 | 90 | 3 | 1834.3 | 56% | 2.47881 | 1.75 | 4.34 | 0.00175 |
| Lungs | 14 | Balloon 10 | Trachea | 18 × 55 | 10886 | 90 | 2 | 8490.3 | 78% | 13.37364 | 0.657 | 8.79 | 0.000657 |
| Lungs | 14 | Balloon 2 | Bronchi | 10 × 30 | 3299 | 90 | 3 | 2509.2 | 76% | 1.35561 | 0 | 0 | 0 |
| Lungs | 14 | Balloon 3 | Bronchi | 8 × 30 | 2639 | 90 | 3 | 1796.2 | 68% | 1.4749 | 14.4 | 21.2 | 0.0144 |
| Lungs | 14 | Balloon 4 | Bronchi | 18 × 55 | 10886 | 90 | 3 | 8543 | 78% | 5.32653 | 4.46 | 23.8 | 0.00446 |
| Lungs | 14 | Balloon 5 | Bronchi | 10 × 30 | 3299 | 90 | 3 | 2543.1 | 77% | 3.30574 | 0.707 | 2.34 | 0.000707 |
| Lungs | 14 | Balloon 6 | Bronchi | 10 × 30 | 3299 | 90 | 3 | 2653 | 80% | 2.02264 | 0 | 0 | 0 |
| Lungs | 14 | Balloon 7 | Bronchi | 8 × 30 | 2639 | 90 | 3 | 1856.8 | 70% | 1.72604 | 4.88 | 8.42 | 0.00488 |
| Lungs | 14 | Balloon 8 | Bronchi | 18 × 55 | 10886 | 90 | 3 | 9312.8 | 86% | 4.60936 | 0 | 0 | 0 |
| Lungs | 14 | Balloon 9 | Trachea | 18 × 65 | 12865 | 90 | 2 | 5564.4 | 43% | 13.98751 | 2.47 | 34.5 | 0.00247 |
| Lungs | 28 | Balloon 1 | Bronchi | 10 × 30 | 3299 | 90 | 3 | 2489.9 | 75% | 1.76904 | 0.331 | 0.586 | 0.000331 |
| Lungs | 28 | Balloon 10 | Trachea | 18 × 65 | 12865 | 90 | 2 | 8503 | 66% | 13.69496 | 2.24 | 30.7 | 0.00224 |
| Lungs | 28 | Balloon 2 | Bronchi | 8 × 30 | 2639 | 90 | 3 | 2259.5 | 86% | 0.98894 | 0 | 0 | 0 |
| Lungs | 28 | Balloon 3 | Bronchi | 14 × 30 | 4618 | 90 | 3 | 3020.8 | 65% | 1.01679 | 0.387 | 0.393 | 0.000387 |
| Lungs | 28 | Balloon 4 | Bronchi | 15 × 55 | 9072 | 90 | 3 | 5940 | 65% | 5.63988 | 18.2 | 103 | 0.0182 |
| Lungs | 28 | Balloon 5 | Bronchi | 12 × 30 | 3958 | 90 | 3 | 2378.8 | 60% | 2.20572 | 0 | 0 | 0 |
| Lungs | 28 | Balloon 6 | Bronchi | 8 × 30 | 2639 | 90 | 3 | 1812.5 | 69% | 1.19696 | 0 | 0 | 0 |
| Lungs | 28 | Balloon 7 | Bronchi | 15 × 55 | 9072 | 90 | 3 | 6714.1 | 74% | 5.29188 | 4.95 | 26.2 | 0.00495 |
| Lungs | 28 | Balloon 8 | Trachea | 18 × 65 | 12865 | 90 | 2 | 6704 | 52% | 12.16796 | 1.08 | 13.1 | 0.00108 |
| Lungs | 28 | Balloon 9 | Bronchi | 8 × 30 | 2639 | 90 | 3 | 1205.8 | 46% | 1.42766 | 6.95 | 9.92 | 0.00695 |

### Local and Non-Target Organ Histopathology at 28 Days

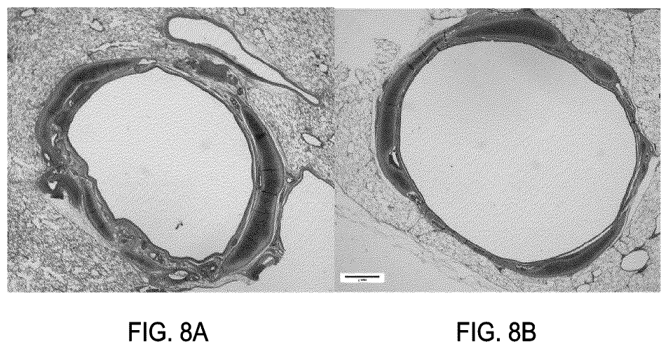

FIG. 8A illustrates a SEM image of a test article treatment site 28 days after treatment. FIG. 8B illustrates SEM image of a control article treatment site after 28 days. At the treatment sites there was no thrombosis, no loss of epithelium, no submucosal hemorrhage, and no necrosis. The airway lumen remained patent with no airway wall collapse. As is expected after treatment with paclitaxel, submucosal inflammation, smooth muscle loss, submucosal fibrosis, and adventitial fibrosis were more common in the test animal than in the control animal. This aligns with the healing responses after exposure to paclitaxel. Under SEM, airways from test animals were >90% covered with ciliated epithelial cells at 28 days versus 51%-90% coverage in airways from control animals.

There were no microscopic morphological abnormalities observed in any of the heart, liver, spleen, kidney, or downstream lung tissue. All of these tissues appeared normal with no device related findings during bread loafing at gross necropsy and during histopathological assessment.

### Overall Preclinical Study Conclusions

The following conclusions were drawn from the preclinical studies: Drug was successfully delivered to the treatment sites; drug concentration in the treatment sites decreases with time to low levels at 28 days; drug concentration within non-target organs is below the limit of quantitation at 28 days; drug concentration within the plasma is below the limit of quantitation at 7 days; there were no gross or histological observations of tracheobronchial tree toxicity arising from the treatments; in all test animals the tracheobronchial tree remained patent, and test animals were able to behave normally as shown by animal observations; and, the DCB performed acceptably and was able to be tracked, inflated, deflated, and withdrawn for all treatments. No major safety concerns have been raised in any of the preclinical studies performed to date with overdosing of 10-12 treatments per animal and no device-associated adverse reactions have been identified.

### Example 3. Acute Preclinical Study: Nasal Tissue

Acute drug transfer in nasal tissue was measured using a 10×30 balloon. Table 8 shows 2-hour paclitaxel drug concentration in the treated tissues (the lateral nasal cavity (Tx 2)). Table 8 demonstrates that paclitaxel can be successfully delivered to nasal tissue on a drug coated balloon.

TABLE 8
Drug transfer in nasal tissue after 2-hours.

| Tissue Weight (g) | Conc. (ng/g) | Total Paclitaxel (ng) |
|---|---|---|
| 0.4345 | 25200 | 10900 |
| 0.91718 | 7910 | 7250 |
| 0.44501 | 7000 | 3120 |
| 1.29058 | 17000 | 21900 |
| 0.65835 | 14300 | 9410 |
| 1.2294 | 9930 | 12200 |

### Example 4. Chronic Preclinical Study: Nasal Tissue

Table 9 shows results of the chronic preclinical study. Nasal Tx 1 was the superior nasal cavity, Nasal Tx 2 was the lateral nasal cavity, and Nasal Tx 3 was anterior and inferior nasal cavity.

TABLE 9
Nasal tissue chronic preclinical study

| | | | | | | | | Treat- | Treat- | | Treat- | | |
|---|---|---|---|---|---|---|---|---|---|---|---|---|---|
| | | | | | | Total | | ment | ment | In- | ment | | |
| | | | | Tissue | | Pac- | | Site | Site | flation | Area | DCB | Stretch |
| Tissue | | | | Weight | Conc. | litaxel | DCB | Width | Height | Pressure | Calc'd | Area | Ratio |
| Type | Term | Subject | Sample ID | (g) | (ng/g) | (ng) | Size | (mm) | (mm) | (atm) | (mm²) | (mm2) | (%) |
| Nasal Tissue | 14 d | 22S0154 | RT Nasal Tx 1 | 0.42982 | 11.2 | 4.81 | 10 × 30 | 4 | 9 | | 28.27 | 78.54 | 177.8 |
| Nasal Tissue | 28 d | 22S0218 | LT Nasal Tx 1 | 0.32844 | 0.962 | 0.316 | 8 × 30 | 3 | 8 | | 18.85 | 50.27 | 166.7 |
| Nasal Tissue | 28 d | 22S0218 | RT Nasal Tx 2 | 0.45434 | 0.469 | 0.213 | 8 × 30 | 3 | 6 | | 14.14 | 50.27 | 255.6 |
| Nasal Tissue | 7 d | 22S0222 | LT Nasal Tx 3 | 0.56979 | 11.4 | 6.50 | 12 × 30 | 5 | 8 | 8 | 31.42 | 113.10 | 260.0 |
| Nasal Tissue | 7 d | 22S0222 | RT Nasal Tx 1 | 0.44883 | 0.448 | 0.201 | 6 × 30 | 3 | 6 | 6 | 14.14 | 28.27 | 100.0 |

### Part II. OXYGEN-1 Human Study.

### 1. INTRODUCTION

### 1.1. Background

Central airway obstruction (CAO) is defined as >50% reduction in cross-sectional area (CSA) of the trachea, mainstem bronchi, bronchus intermedius or a lobar bronchus.

CAO can result from a variety of disease processes. Acquired benign CAO is most frequently caused by iatrogenic tracheal trauma after prolonged intubation or tracheostomy tube placement. It is estimated that the incidence of post intubation tracheal stenosis (PITS) ranges from 6% to 21%, post tracheostomy tracheal stenosis (PTTS) 0.6% to 21%, and post-transplantation from 5% to 30%. Recently, the incidence of this disorder as post-COVID of PITS appears to be rising because current pandemic.

Severe stenosis (>50% reduction in CSA) causes symptoms and may warrant prompt intervention. Benign CAO is responsible for significant morbidity due to dyspnea and impaired quality of life (QOL).

Management of CAO remains a challenge and often requires recurrent procedures. The current knowledge regarding the most effective way to treat tracheal stenosis is inconclusive. Endoluminal therapy either by dilatation, scar tissue resection or stent insertion is an established treatment for benign airway stenosis although the surgical approach is still considered the “gold standard”. For lesions causing impending respiratory failure, surgical resection or stent placement is the most prudent treatment and in less urgent conditions, bronchoscopic balloon dilation (BBD) has been considered a simple, rapid, and safe method to restore airway caliber. BBD has been used alone or in combination with other modalities as palliative therapy in patients who are ineligible for surgical treatment or who develop post-surgical re-stenosis, most done on iatrogenic stenoses, which are typically soft. BBD are short in time, can be performed on a day-service, and are considered highly safe compared with open surgery.

Drug delivery directly into the stenotic lesion aiming to minimize recurrence rates and prolong the symptom-free period after the intervention has been gaining traction in the last decade. The most used as co-adjuvant are corticosteroid and mitomycin-c (MMC). The overall results of the use of MMC for airway stenosis treatment in humans are conflicting. Antineoplastic agents 5-fluorouracil, paclitaxel, and cisplatin have proven safe when injected directly into the airway and are still under investigation.

### 1.2. Rationale for Study

Paclitaxel coated balloon offers both mechanical dilation of airway obstruction and local drug effect for underlying cell hyperplasia (2-in-1 device). There are two early clinical results of paclitaxel drug coated balloon off-label use in benign CAO giving encouraging initial findings. It is hypothesized that Airiver's pulmonary paclitaxel coated balloon (DCB) will improve patient outcome in the bronchoscopic treatment of benign airway stenosis, and, as an adjunct to standard of care, will prolong airway patency compared to the standard of care alone.

### 2. MATERIALS AND METHODS, STUDY DESIGN, AND RESULTS

### 2.1. Device Description

The Airiver Pulmonary Drug Coated Balloon Dilation Catheter was designed to be compatible with the working channel of commonly used bronchoscopes and could be delivered through-the-scope (TTS). It was an over-the-wire catheter compatible with 0.035″ guidewire. It had a tapered atraumatic tip. The distal end of the catheter had a semi-compliant inflatable balloon coated with a proprietary coating containing the drug paclitaxel and carriers that facilitated the drug's transfer to the tracheal/bronchial wall upon inflation.

The device was be available in balloon diameters to cover diameters from 8 to 20 mm and balloon lengths of 30 mm, 55 mm, and 65 mm with either 75 cm, 110 cm, or 240 cm shaft length (Table 10). The device had two radiopaque marker bands indicating drug coated working length (WL) when seen under fluoroscopy. The device was provided sterile and was intended for single-use only. The device is illustrated in FIG. 5.

All materials used in the construction of the Airiver Pulmonary DCB are known biocompatible materials and are commonly used in the medical device industry, with the same coating dose of paclitaxel (3.5 mg/mm) as the commercial Optilume DCB for non-vascular indication. The drug coating included paclitaxel and an excipient as a homogeneous blend. The excipient was pentraerythritol ethoxlate (PEE) 15/4. The weight ratio of the excipient to paclitaxel in the coating was 1:3. The devices had the same diameters as commercially available pulmonary dilatation balloon catheters. Details of the Airiver Pulmonary DCB are shown in Table 10.

TABLE 10
AIRIVER Pulmonary Balloon Catheter.

| Balloon | | Nominal | NOMINAL | MIN | | |
|---|---|---|---|---|---|---|
| Size | Nominal | Balloon | BALLOON | RATED | | |
| (diameter × | Pressure | Diameter | LENGTH | BURST | Shaft | Bronchoscope |
| length) | (atm) | (mm) | (mm) | (ATM) | Length | Compatibility |
| 6 × 30 | 6 | 6.0 | 30.0 | 12 | 75 cm | ≥2.8 mm |
| 8 × 30 | 6 | 8.0 | | 12 | | Working |
| 10 × 30 | 6 | 10.0 | | 10 | | Channel |
| 12 × 30 | 6 | 12.0 | | 8 | | ≤60 cm Total |
| 14 × 30 | 6 | 14.0 | | 8 | | Length |
| 4 × 30 | 3 | 4.0 | | 13 | 110 cm | |
| 6 × 30 | 3 | 6.0 | | 12 | | |
| 8 × 30 | 3 | 8.0 | | 11 | | |
| 10 × 30 | 3 | 10.0 | | 10 | | |
| 12 × 30 | 2 | 12.0 | | 9 | | |
| 12 × 55 | 2 | 12.0 | 55.0 | 9 | | |
| 15 × 55 | 2 | 15.0 | | 8 | | |
| 18 × 55 | 2 | 18.0 | | 7 | | |
| 12 × 65 | 3 | 12.0 | 65.0 | 7 | 240 cm | |
| 15 × 65 | 3 | 15.0 | | 6 | | |
| 18 × 65 | 3 | 18.0 | | 5 | | |

### 2.2. Demographics

Table 11 presents the subject demographics and risk factors. The mean age of this cohort was 48.4 years of age, with 60% of the subjects being male.

TABLE 11
Subject Demographics

| | Subjects (N = 50) |
|---|---|
| | n/N (%) |
| Age (years), Mean ± SD, Min Max, (n) | 48.4 ± 16.5, 18 81, (50) |

Gender-n/N (%)

| | |
|---|---|
| Male | 30/50 (60.0%) |
| Female | 20/50 (40.0%) |

Race/Ethnic Origin of Subjects-n/N (%)

| | |
|---|---|
| Hispanic/Latino | 35/50 (70.0%) |
| White/Caucasian | 15/50 (30.0%) |
| Height (cm), Mean ± SD, Min Max, (n) | 168.2 ± 10.5, 145 186, (49)* |
| Weight (kg), Mean ± SD, Min Max, (n) | 87.97 ± 22.08, 49.7 142.0, (49)* |
| BMI (kg/m²), Mean ± SD, Min Max, (n) | 31.01 ± 6.87, 18.1 54.1, (49)* |

*Baseline height and weight measurements are missing for Subject 02-017.

### 2.3. Clinical Endpoints

### 2.3.1. Primary Safety Endpoint

The primary safety endpoint of the study was freedom from major adverse events (MAE) post index procedure through 30 days. The primary safety was assessed by calculating the proportion of subjects free from the MAE at 30 days aggregated over the subjects. As of the data cutoff date, there was no MAE event that occurred in any subject 30 days after the index procedure. The freedom from MAE at 30 days was therefore 100%.

### 2.3.2. Primary Efficacy Endpoint

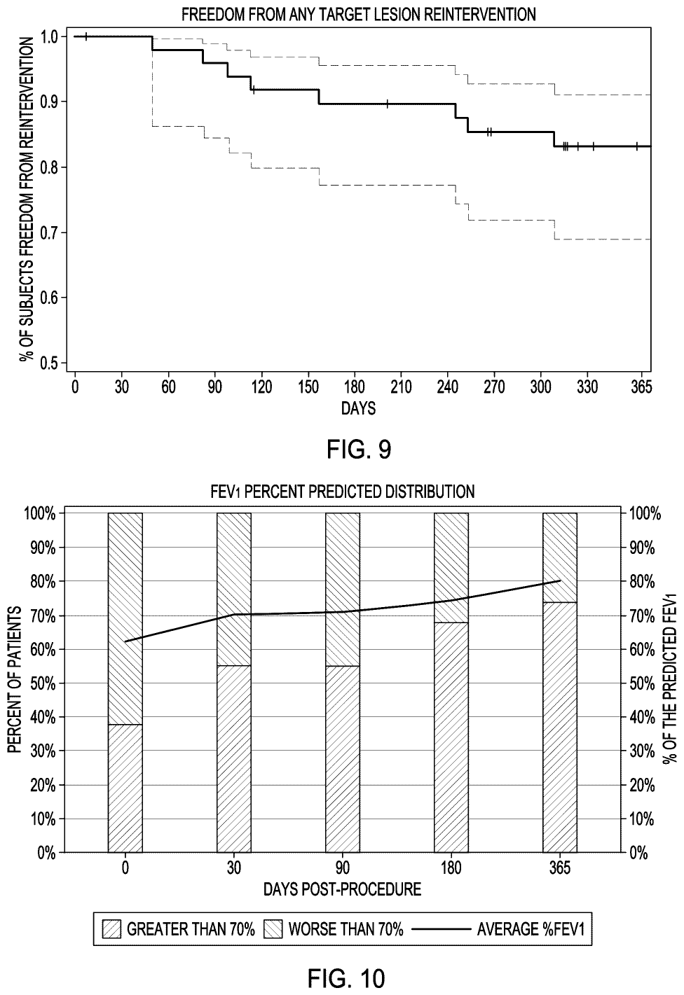

The primary efficacy endpoint of the study was freedom from symptom-driven target lesion reintervention due to recurrence of stenosis through 6 months. As of the data cutoff date, five (5) subjects had a reintervention due to worsening symptoms or stenosis recurrence within 6 months after their procedure. This resulted in an 86% freedom from reintervention rate through 6 months per Kaplan-Meier assessment. Through 12 months, eight (8) subjects had reintervention due to worsening symptoms or stenosis recurrence. Percentage freedom from target lesion reintervention versus days is illustrated in FIG. 9.

### 2.3.3. Secondary Endpoint 1

The secondary endpoint 1 was the incidence of, and time to, symptom-driven target lesion reintervention through 12 months. The incidence of symptom-driven target lesion reintervention through 12 months was assessed by calculating the proportion of subjects with symptom-driven target lesion reintervention through at each follow-up through 12 months. Table 12 presents the incidence of symptom-driven target lesion reintervention at each follow-up.

TABLE 12
Incidence of Target Lesion Reintervention through 12 Months.

| Parameter | Visit | Subjects (N = 50) n/N (%) |
|---|---|---|
| Incidence of any target lesion | 1 Month | 0/49 (0%) [0.0%, 7.3%] |
| reintervention | 3 Months | 2/49 (4.1%) [0.5%, 14.0%] |
| | 6 Months | 5/48 (10.4%) [3.5%, 22.7%] |
| | 12 Months | 8/35 (22.9%) [10.4%, 40.1%] |
| Incidence of symptom-driven | 1 Month | 0/49 (0%) [0.0%, 7.3%] |
| target lesion reintervention | 3 Months | 1/49 (2.0%) [0.1%, 10.9%] |
| | 6 Months | 2/47 (4.3%) [0.5%, 14.5%] |
| | 12 Months | 4/31 (12.9%) [3.6%, 29.8%] |
| Incidence of image-driven | 1 Month | 0/49 (0%) [0.0%, 7.3%] |
| target lesion reintervention | 3 Months | 1/49 (2.0%) [0.1%, 10.9%] |
| | 6 Months | 5/48 (10.4%) [3.5%, 22.7%] |
| | 12 Months | 6/35 (17.1%) [6.6%, 33.6%] |

The confidence intervals are calculated using the so-called “exact” confidence intervals, computed by the method of Clopper and Pearson (Biometrika 26:404-413, 1934), which is based on a relationship between the F distribution and the binomial distribution.

### 2.3.4. Secondary Endpoint 2.

The secondary endpoint 2 was bronchoscopic target lesion patency (Myer-Cotton airway grading system) change through 12 months. Table 13 shows the distribution of grades of the target stenoses at each timepoint. At baseline, none of the stenoses were Grade 1 (<50% stenosis) versus between 77.8% and 85.7% of stenoses being Grade 1 at the follow-ups through 12 months. When compared to baseline, the distribution of stenoses grades was statistically significant at all follow up timepoints.

TABLE 13
Myer-Cotton Grading through 12 Months.

| | Target Lesions (N = 53) n/N (%) | | | | |
|---|---|---|---|---|---|
| | Baseline | 1 Month | 3 Months | 6 Months | 12 Months |
| Grade 1 | 0/53 (0.0%) | 42/49 (85.7%) | 42/51 (82.4%) | 35/45 (77.8%) | 18/22 (81.8%) |
| Grade 2 | 19/53 (35.8%) | 4/49 (8.2%) | 7/51 (13.7%) | 6/45 (13.3%) | 2/22 (9.1%) |
| Grade 3 | 30/53 (56.6%) | 3/49 (6.1%) | 2/51 (3.9%) | 4/45 (8.9%) | 2/22 (9.1%) |
| Grade 4 | 4/53 (7.5%) | 0/49 (0.0%) | 0/51 (0.0%) | 0/45 (0.0%) | 0/22 (0.0%) |

‘Grade 1’ was assigned to lesions with no re-stenosis observed by the physician.

When looking at change from baseline in Myer-Cotton grading for each individual, the percentage of subjects with improvement at least one grade was 91.8%, 94.1%, 88.9%, and 86.5% at 1, 3, 6, and 12 months, respectively. The improvement in Myer-Cotton grading through 12 months is shown in Table 14.

TABLE 14
Improvement Myer-Cotton Grading through 12 Months.

| | Target Lesions (N = 53) n/N (%) | | | | |
|---|---|---|---|---|---|
| | Worsened | No Change | Improved +1 | Improved +2 | Improved +3 |
| 1 Month | 0/49 (0.0%) | 4/49 (8.2%) | 19/49 (38.8%) | 22/49 (44.9%) | 4/49 (8.2%) |
| 3 Months | 0/51 (0.0%) | 3/51 (5.9%) | 21/51 (41.2%) | 25/51 (49.0%) | 2/51 (3.9%) |
| 6 Months | 0/45 (0.0%) | 5/45 (11.1%) | 18/45 (40.0%) | 21/45 (46.7%) | 1/45 (2.2%) |
| 12 Months | 1/22 (4.5%) | 2/22 (9.1%) | 9/22 (40.9%) | 10/22 (45.5%) | 0/22 (0.0%) |

### 2.3.5. Secondary Endpoint 3.

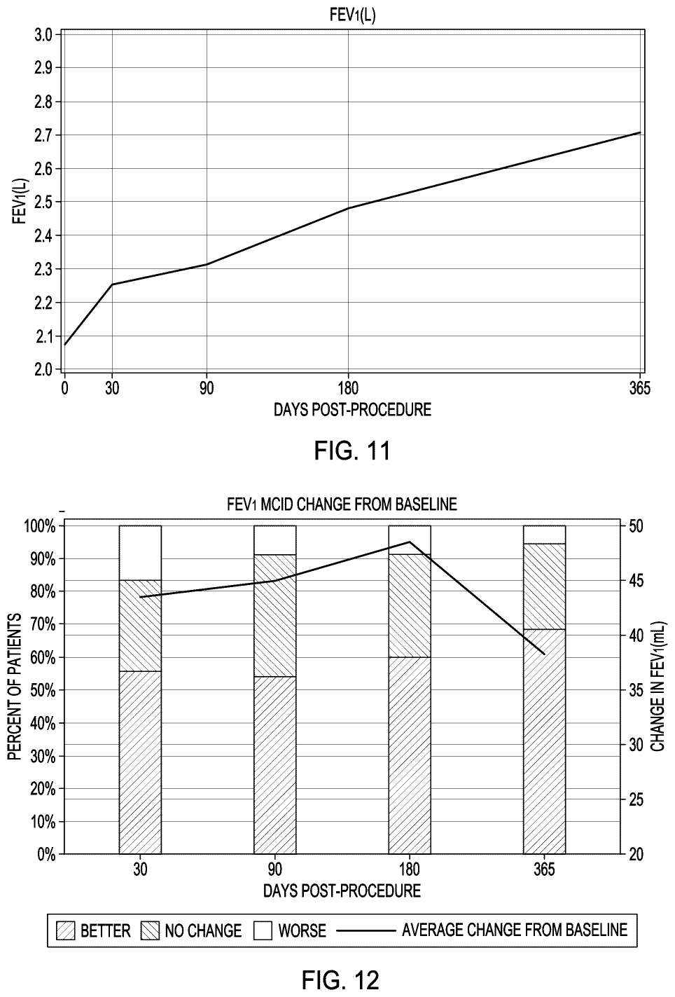

The secondary endpoint 3 was spirometry change through 12 months. Table 15 summarizes the change in spirometry measures from baseline through 12 months post-procedure. FEV₁, PEF, and FEV₁/FVC all showed improvement starting at 1 month and maintained that improvement, or improved more, through 12 months, which shows improved lung function in these patients after treatment with the DCB. FIG. 10 illustrates the FEV1 percent predicted distribution versus days post-procedure. FIG. 11 shows the mean FEV₁ (L) change versus days post-procedure.

TABLE 15
Change in Lung Function through 12 Months.

| | Subjects (N = 50) | | | | |
|---|---|---|---|---|---|
| | Baseline | 1 Month | 3 Months | 6 Months | 12 Months |
| FVC (L) | 3.40 ± 1.05, | 3.27 ± 1.12, | 3.36 ± 1.04, | 3.55 ± 0.99, | 3.72 ± 1.18, |
| | 1.6 6.2, (37) | 1.1 6.1, (47) | 1.2 6.0, (49) | 1.9 6.1, (44) | 1.8 6.1, (19) |
| % | 82.92 ± 19.35, | 78.69 ± 21.67, | 83.25 ± 16.29, | 86.53 ± 13.14, | 89.54 ± 16.07, |
| Predicted | 39.0 130.0, (37) | 20.0 119.0, (47) | 38.0 117.0, (49) | 60.0 117.0, (44) | 59.0 120.0, (19) |
| FVC (%) | | | | | |
| FEV1 (L) | 2.07 ± 0.92, | 2.25 ± 0.92, | 2.32 ± 0.97, | 2.48 ± 0.95, | 2.71 ± 0.98, |
| | 0.2 4.1, (37) | 0.5 4.1, (47) | 0.6 4.3, (49) | 0.6 4.3, (44) | 1.2 4.2, (19) |
| % | 62.00 ± 25.29, | 70.09 ± 21.09, | 70.92 ± 22.98, | 74.26 ± 21.08, | 80.11 ± 15.66, |
| Predicted | 8.0 108.0, (37) | 21.0 109.0, (47) | 26.0 119.0, (49) | 22.0 118.0, (44) | 52.0 120.0, (19) |
| FEV1 (%) | | | | | |
| FEV1/FVC | 61.08 ± 22.28, | 68.11 ± 14.76, | 68.60 + 17.66, | 69.06 ± 15.73, | 72.66 ± 11.68, |
| (%) | 14.6 92.3, (37) | 27.6 97.6, (47) | 25.1 116.8, (49) | 20.7 88.9, (44) | 51.8 88.6, (19) |
| PEF | | | | | |
| (L/sec) | 3.58 ± 1.80, 0.6 | 4.33 ± 1.98, 1.0 | 4.57 ± 2.14, 1.1 | 4.79 ± 2.12, 0.5 | 5.08 ± 2.11, 1.7 |
| | 6.7, (24) | 7.8, (25) | 9.4, (30) | 8.1, (30) | 9.2, (18) |

When looking at the change from baseline in FEV1, 68.4% of subjects had an increase above the MCID (>100 ml) at 12 months. At 1-month post-procedure this percentage was at 55.6% and it steadily increased through 12 months. Table 16 shows distribution of FEV₁ response categories over time. FIG. 12 illustrates FEV₁ MCID change from baseline versus days post-procedure.

TABLE 16
Distribution of FEV1 Response Categories Over Time.

| Subjects (N = 50) n/N (%) | | | |
|---|---|---|---|
| 1 Month | 3 Months | 6 Months | 12 Months |

Change from Baseline in FEV₁ − n/N (%)

| | | | | |
|---|---|---|---|---|
| >100 mL | 20/36 (55.6%) | 20/37 (54.1%) | 21/35 (60%) | 13/19 (68.4%) |
| −100 to 100 mL | 10/36 (27.8%) | 14/37 (37.8%) | 11/35 (31.4%) | 5/19 (26.3%) |
| <−100 mL | 6/36 (16.7%) | 3/37 (8.1%) | 3/35 (8.6%) | 1/19 (5.3%) |

When looking at Peak Expiratory Flow (PEF), 92.9% of subjects had an increase above the MCID (18.8 L/min) at 12 months when comparing to baseline. Importantly, looking at deterioration of PEF after the post-treatment baseline value at 1 month, only 21.4% had a decrease more than the MCID and only 7.1% had a decrease greater than 15%. At the 3-month timepoint, 5 subjects (20.8%) had a deterioration greater than 15%, which signifies a potential need for reintervention. Of these 5 subjects, 4 did end up having a reintervention. Table 17 shows the distribution of PEF response categories over time.

TABLE 17
Distribution of PEF Response Categories Over Time.

| Subjects (N = 50) n/N (%) | | | |
|---|---|---|---|
| 1 Month | 3 Months | 6 Months | 12 Months |

Change from Baseline in PEF - n/N (%)

| | | | | | |
|---|---|---|---|---|---|
| >18.8 | L/min | 12/22 (54.5%) | 16/24 (66.7%) | 15/22 (68.2%) | 13/14 (92.9%) |
| −18.8 to 18.8 | L/min | 5/22 (22.7%) | 3/24 (12.5%) | 2/22 (9.1%) | 1/14 (7.1%) |
| <−18.8 | L/min | 5/22 (22.7%) | 5/24 (20.8%) | 5/22 (22.7%) | 0/14 (0.0%) |

Change from 1 Month in PEF - n/N (%)

| | | | | |
|---|---|---|---|---|
| >18.8 | L/min | 13/24 (54.2%) | 11/23 (47.8%) | 9/14 (64.3%) |
| −18.8 to 18.8 | L/min | 3/24 (12.5%) | 4/23 (17.4%) | 2/14 (14.3%) |
| <−18.8 | L/min | 8/24 (33.3%) | 8/23 (34.8%) | 3/14 (21.4%) |

Percent Change from 1 Month in PEF - n/N (%)

| | | | |
|---|---|---|---|
| >15% | 9/24 (37.5%) | 9/23 (39.1%) | 7/14 (50.0%) |
| −15% to 15% | 10/24 (41.7%) | 10/23 (43.5%) | 6/14 (42.9%) |
| <−15% | 5/24 (20.8%) | 4/23 (17.4%) | 1/14 (7.1%) |

Expiratory disproportion index (EDI) is an indicator of potential central airway obstruction if the value is greater than 50. In this study, 75% of subjects had an EDI greater than 50 at baseline, which reduced to 61.1% of subjects at the 12-month timepoint. Table 18 shows the proportion of patients with expiratory disproportion index (EDI) above and below 50 over time.

TABLE 18
Proportion of Patients with Expiratory disproportion
index (EDI) Above and Below 50 Over Time.

| | Subjects (N = 50) n/N (%) | | | | |
|---|---|---|---|---|---|
| | Baseline | 1 Month | 3 Months | 6 Months | 12 Months |
| EDI > 50 | 18/24 (75.0%) | 17/25 (68.0%) | 19/30 (63.3%) | 19/30 (63.3%) | 11/18 (61.1%) |
| EDI ≤ 50 | 6/24 (25.0%) | 8/25 (32.0%) | 11/30 (36.7%) | 11/30 (36.7%) | 7/18 (38.9%) |

### 2.3.6. Secondary Endpoint 4.

The secondary endpoint 4 was mMRC (Modified Medical Research Council) dyspnea score change through 12 months. Subjects were asked about their shortness of breath using the Modified Medical Research Council (mMRC) dyspnea scale, shown in Table 19.

TABLE 19
Definition of the mMRC Dyspnea Score.

| Grade | Dyspnea Related to Activity |
|---|---|
| 0 | I only get breathless with strenuous exercise |
| 1 | I get short of breath when hurrying on level ground |
| | or walking up a slight hill |
| 2 | On level ground, I walk slower than people of my age |
| | because of breathlessness, or I have to stop for breath |
| | after walking at my own pace on level ground |
| 3 | I stop for breath after walking about 100 meters or |
| | after a few minutes on level ground |
| 4 | I am too breathless to leave the house or I am breathless |
| | when dressing / undressing |

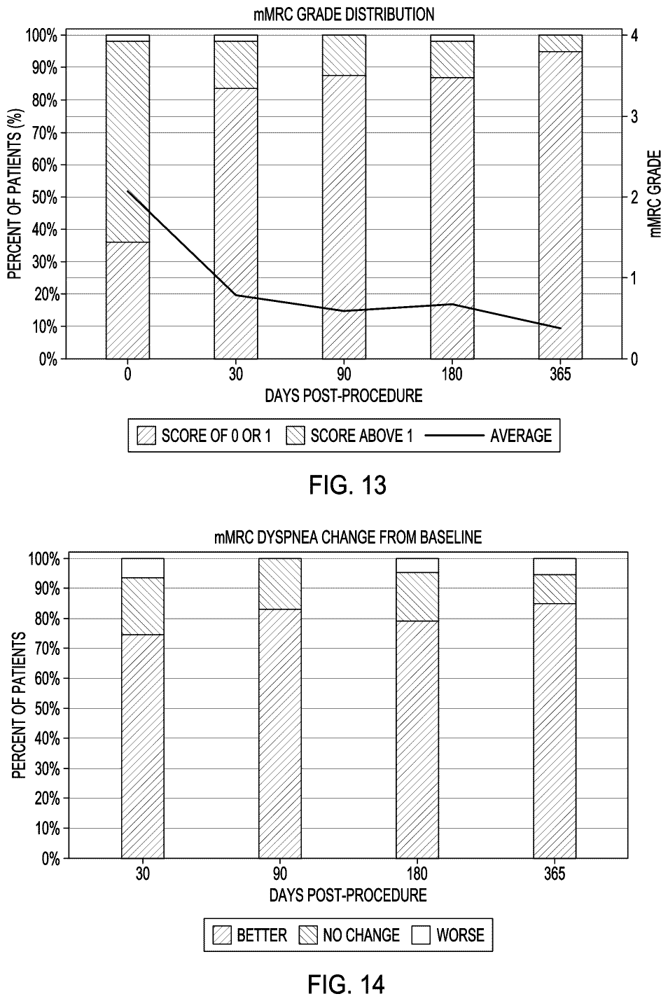

Table 20 presents the modified medical research council (mMRC) dyspnea scores through 12 months. The subjects had an average mMRC score of 2.1 at baseline, which improved to 0.4 at 12 months post procedure. FIG. 13 illustrates mMRC dyspnea grade distribution versus days post-procedure.

TABLE 20
mMRC Dyspnea Score through 12 Months.

| | Subjects (N = 50) n/N (%) | | | | |
|---|---|---|---|---|---|
| | Baseline | 1 Month | 3 Months | 6 Months | 12 Months |
| Grade 0 | 3/49 (6.1%) | 25/48 (52.1%) | 30/49 (61.2%) | 25/45 (55.6%) | 16/21 (76.2%) |
| Grade 1 | 15/49 (30.6%) | 16/48 (33.3%) | 13/49 (26.5%) | 15/45 (33.3%) | 4/21 (19.0%) |
| Grade 2 | 12/49 (24.5%) | 3/48 (6.3%) | 3/49 (6.1%) | 1/45 (2.2%) | 0/21 (0.0%) |
| Grade 3 | 13/49 (26.5%) | 0/48 (0.0%) | 2/49 (4.1%) | 3/45 (6.7%) | 0/21 (0.0%) |
| Grade 4 | 6/49 (12.2%) | 4/48 (8.3%) | 1/49 (2.0%) | 1/45 (2.2%) | 1/21 (4.8%) |

Baseline mMRC Dyspnea Score is missing for Subject 01-001.

Table 21 shows the distribution of the change per subject in mMRC over the study period. At 12 months post procedure, 85% of subjects had an improvement of at least 1 grade. FIG. 14 illustrates mMRC dyspnea change from baseline versus days post-procedure.

TABLE 21
Distribution of mMRC Dyspnea Score Response Categories Over Time.

| Subjects (N = 50) n/N (%) | | | |
|---|---|---|---|
| 1 Month | 3 Months | 6 Months | 12 Months |

Change from Baseline in mMRC Dyspnea Score - n/N (%)

| | | | | |
|---|---|---|---|---|
| ≥1 Grade Improvement | 36/48 (75.0%) | 40/48 (83.3%) | 35/44 (79.5%) | 17/20 (85.0%) |
| No Change | 9/48 (18.8%) | 8/48 (16.7%) | 7/44 (15.9%) | 2/20 (10.0%) |
| ≥1 Grade Worsened | 3/48 (6.3%) | 0/48 (0.0%) | 2/44 (4.5%) | 1/20 (5.0%) |

### 3. DISCUSSIONS AND OVERALL CONCLUSION

The OXYGEN-1 study was a prospective, non-randomized, multi-center study to assess the initial safety and efficacy of the Airiver Medical Pulmonary DCB in the treatment of benign central airway obstructions.

Endoscopic dilation has become the standard of care for management of benign central airway obstruction. Adequate dilation and relief of symptoms can typically be achieved through endoscopic treatment alone; however, recurrence of the obstruction is common, occurring in up to 80% of subjects.

The technology of the Airiver Medical Pulmonary DCB combines the advantages of minimally invasive dilation with local paclitaxel delivery to the treated tissue to reduce the rate of stenosis recurrence. The cohort of subjects evaluated in the OXYGEN-1 study showed that dilation with the Airiver DCB provided immediate symptom relief, with the mean mMRC score reduced and mean spirometry values (FEV₁ and PEF, specifically) increased from baseline to 30 days. Evidence of drug effects was also observed, as symptom and functional improvement was maintained through 12 months. This study shows the benefit of paclitaxel coated DCBs for treatment of airway obstructions.

There were no device related series adverse events (SAEs) in this study, with three procedure related SAEs, including no incidence of protocol defined major adverse event (MAE). All three procedure-related SAEs would be expected per standard of care endoscopic dilation of airway obstructions.

### Part III. Sinus Clinical Trial.

### Protocol for Use of Sinus Balloon.

The following is the general procedure used to treat chronic rhinosinusitis (CRS) with the Airiver DCB device in this Part, unless otherwise described. 1) The target treatment location (lesion) was identified. 2) The standard of care preparation of the lesion was performed, including any surgical dissection, irrigation, or balloon dilation with uncoated balloons. 3) A 0.035″ guidewire was optionally positioned across the lesion. 4) An appropriate device DCB size was selected, with the inflated balloon diameter 2-3 mm larger than the prepared lesion to ensure there is enough pressure to dilate the tissue and transfer the drug into the surrounding tissue. 5) The inflation device was attached to the catheter hub. 6) The DCB was prepared by evacuating air from the balloon and replacing it with saline or sterile water. The drug coating was hydrated by soaking the balloon in sterile saline or water for 1 to 2 min. The balloon was not yet inflated. 7) The balloon was tracked and positioned across the lesion under endoscopic visualization to ensure proper placement. The balloon was advanced directly into position, or alternatively the balloon was delivered through a cannulated surgical instrument or over a guidewire. 8) The inflation device was used to inflate the balloon with normal saline or sterile water to the desired pressure, without exceeding the rated burst pressure (RBP) of the balloon. 9) The pressure was monitored and the waist of the balloon was watched to determine complete balloon expansion. 10) Final dilation diameter and pressure was maintained for 2 minutes to ensure adequate compression of edematous tissue and drug transfer to the tissue. 11) The balloon was deflated by applying vacuum to the balloon with the inflation device. It was ensured that the balloon was deflated before attempting withdrawal. When the balloon was completely deflated, the guidewire (if applicable) and DCB were slowly withdrawn under endoscopic visualization.

### 1. BACKGROUND INFORMATION

### 1.1. Chronic Rhinosinusitis

Chronic rhinosinusitis (CRS) is a multifactorial inflammatory disease of the nasal passages and paranasal sinuses. It affects 2%-16% of the US population. According to the International Consensus Statement on Rhinosinusitis, CRS is defined by two or more symptoms (nasal blockage/obstruction/congestion or nasal discharge with or without facial pain or reduction or loss of smell for ≥12 weeks) and associated with signs via nasal endoscopy or CT scan.

Patients with CRS have significant morbidity and reduced health-related quality of life (HRQOL). The burden of CRS is greater than chronic heart failure on social functioning. The US national health care costs of CRS remain very high, at an estimated $8.6 billion per year.

First line treatment of CRS is medical, usually including a nasally applied steroid, supplemented with a combination of antihistamines, antibiotics and/or oral steroids. Surgical interventions (functional endoscopic sinus surgery, FESS, or ESS) are indicated when there is a failure of a prolonged course of medical treatment, with the goal of reestablishing ventilation and mucociliary clearance of the sinuses. Additionally, standard sinus surgery increases distribution of topical therapies to all sinuses. In appropriate patients, balloon sinus dilation (balloon sinuplasty, or BSP) may be performed in lieu of or in conjunction with ESS as a hybrid procedure, and can improve short-term QOL outcomes, per clinical consensus statement. Intraoperative placement of steroid releasing implants improves short-term (30-day) outcomes.

Despite the availability of ESS and the more recent developments of balloon sinuplasty and steroid releasing implants, the existing treatment paradigm has significant limitations. In a large database assessment of 29,934 patients who underwent ESS during the years 1996 to 2016, the long-term revision rate was 15.9% with a mean number of sinus surgeries per patient of 1.22. For balloon dilation of the frontal sinus performed during the years 2008 to 2019, overall revision rates of 15.7% were reported, including revision rates of 26.8% in balloon-only procedures. There is a need for innovative medical therapies to mitigate this high recurrence and reduce the burden of CRS.

In a study of surgical causes of ESS failure, maxillary sinus ostium stenosis was observed in 27%, frontal sinus ostium stenosis was observed in 25%, and adhesions were observed in 56% of the patients who underwent revision ESS. Steroid releasing implants reduce the incidence of adhesions during the first 30 days of wound healing, however a study of the postoperative healing process in 328 patients undergoing FESS revealed first observations of hyperplasia and adhesion of connective tissue at 3-10 weeks postoperatively. A device that mechanically dilates paranasal spaces to reventilate the sinuses and improve distribution of topical medical therapy and which secondarily medically affects the postoperative healing process beyond 30 days offers potential to improve outcomes for CRS patients.

### 2. DEVICE DESCRIPTION

The Airiver Galaxy Drug-Coated Sinus Dilation Balloon (DCB) used in this Part was a combination (device-drug) product. The Galaxy DCB was intended to dilate and locally deliver antiproliferative medication to maintain patency of the paranasal sinus ostia and outflow tracts, including maxillary ostia/ethmoid infundibula, surgically resected ethmoid cavities, frontal ostia/recesses, and sphenoid sinus ostia in patients ≥18 years of age. The sinus ostia and outflow tracts were remodeled by balloon displacement of adjacent tissue and bone. The primary mode of action was mechanical balloon dilation of the paranasal structures. Secondarily, the device delivered an antiproliferative drug, paclitaxel, to the newly dilated space to inhibit hyperactive cell proliferation and fibrotic scar generation following balloon dilation.

### 2.1. Balloon Dilation Catheter.

The Airiver Galaxy DCB was a rapid exchange catheter with a semi-compliant sinus dilation balloon. The distal end of the Galaxy DCB included an atraumatic tip and semi-compliant inflatable balloon, meaning the diameter of the balloon increased slightly with increasing inflation pressure. Table 22 provides the planned sizes for the Airiver Galaxy DCB. The balloon is coated with a proprietary coating of paclitaxel and an excipient, as described in Section 2.2.

TABLE 22
Galaxy DCB Size Matrix.

| Galaxy DCB | Diameter | Maximum Inflation |
|---|---|---|
| Labeled diameter × length (mm) | at (MP) | Pressure (MP) |
| Diameter at Maximum Pressure (MP) | mm | atm |
| 7 × 20 | 7 | 12 |
| 10 × 20 | 10 | 12 |
| 12 × 20 | 12 | 10 |
| 14 × 20 | 14 | 8 |
| 17 × 20 | 17 | 7 |
| 20 × 20 | 20 | 6 |

### 2.2. Drug Coating.

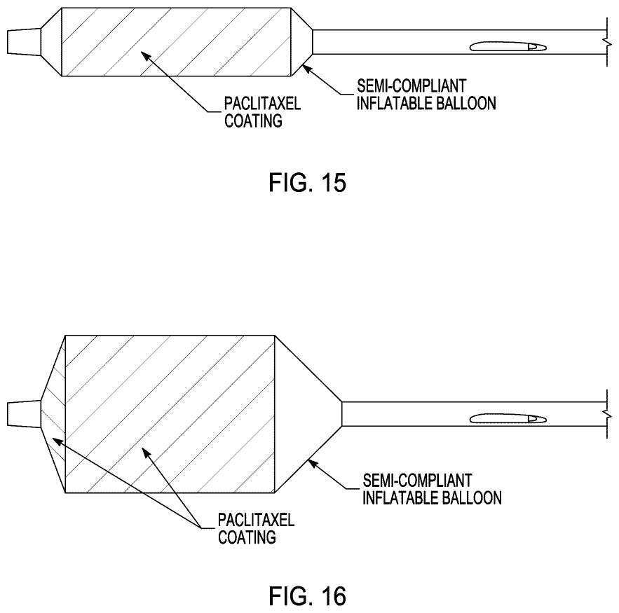

The sinus dilation balloon was coated with a proprietary coating of paclitaxel (API) and an excipient. The drug coating was evenly applied to the cylindrical area between the balloon cones. For the 17 mm and 20 mm diameter Galaxy DCBs, the distal cones were also coated. These larger balloons were typically used in surgically resected ethmoid cavities where the distal cones may reach the posterior aspect of the resected cavities. FIG. 15 illustrates the DCB with drug coating on the cylindrical area between the balloon cones (7, 10, 12, and 14 mm Galaxy DCB). FIG. 16 illustrates the DCB with drug coating on the cylindrical area between the balloon cones and on the distal cone (7, 10, 12, and 14 mm Galaxy DCB). The hatch marked areas in FIG. 15 and FIG. 16 represent the drug coated portions of the balloons.

The drug coating dose density (drug mass per balloon surface area) was 3.5 μg paclitaxel/mm². The excipient was pentaerythritol ethoxylate (PEE) 15/4. The weight ratio of the excipient to paclitaxel in the coating was 1:3. The excipient and the paclitaxel were a homogeneous blend in the coating. PEE is a compound made by attaching polyethylene glycol (PEG) to the available hydroxyl (—OH) groups of pentaerythritol (PEGylation). PEE is a hydrophilic material whose function in the drug coating is to promote even coating film formation and to facilitate coating hydration allowing drug release from the balloon catheter during inflation. The PEG-like structure of PEE is inert and passes through the body safely.

### 2.3. Drug Dosing.

The dose density for each catheter was 3.5 μg paclitaxel/mm² of balloon surface when the balloon was inflated to its nominal pressure. The label claim for each device size is provided in Table 23.

TABLE 23
Galaxy DCB Drug Dose Label Claim.

| | | Label Claim |
|---|---|---|
| Galaxy DCB | Drug Dose Density* | Drug Dose |
| dia. × length (mm) | (μg paclitaxel/mm²) | (mg Paclitaxel) |
| 7 × 20 | 3.5 | 1.3 |
| 10 × 20 | 3.5 | 1.8 |
| 12 × 20 | 3.5 | 2.2 |
| 14 × 20 | 3.5 | 2.6 |
| 17 × 20 | 3.5 | 3.9 |
| 20 × 20 | 3.5 | 4.9 |

*Note:
during manufacturing, the drug coating is applied with the balloon inflated to nominal pressure (3 atm). The balloon is labeled with the diameter at maximum pressure (MP) which is consistent with the labeling of commercially available sinus dilation balloons.

### 2.4. Primary Mode of Action (PMOA).

The Galaxy DCB's primary mode of action was dilation of the narrowed sinus ostium and drainage pathway. During treatment, the catheter was advanced into the narrowed anatomy and inflated, mechanically dilating the paranasal sinus ostium and adjacent drainage structures by compression of edematous mucosa and displacement of underlying bone, to improve drainage and aeration.

Notably, the frontal, anterior ethmoid, and maxillary sinuses all drain through the middle meatus and resected ethmoid cavity after ethmoidectomy, making the patency of these paranasal structures particularly important. Occlusion of the middle meatus by lateralization and scarring of the middle turbinate has been observed in 47.7% of patients in failed endoscopic frontal sinus surgery, and in one study, 26% of subjects who underwent FESS had early synechia removed during postoperative debridements. Much effort has been dedicated to preventing lateralization and scarring of the middle turbinate in sinus surgery, including Bolgerization, conchopexy, metal clips, silastic splints, and partial resection of the middle turbinate. Balloon dilation of the resected ethmoid cavity after ethmoidectomy offers a means of medializing the middle turbinate with uniform pressure along the length of the turbinate, potentially reducing the risk of mucosal injury to its lateral surface and the consequent risk of scarring. Balloon dilation of a surgically resected ethmoid cavity flattens residual bony partitions to create a smoother, more cavernous space, which was observed in endoscopic images from the RESTORE-1 FIH trial. A larger, smoother ethmoid cavity is less likely to form adhesions, effects a larger drainage pathway, and promotes better distribution of topical medication to all sinuses after surgery.

Sinus dilation with the Airiver Sinus DCB was intended to be effective as a standalone procedure. However, in many patients the dilation may be performed in conjunction with ESS or hybrid procedures.

### 2.5. Ancillary Mode of Action.

The Airiver Sinus DCB delivered a small dose of the anti-proliferative drug paclitaxel to the newly dilated tissue during the dilation process to inhibit hyperactive cell proliferation and scar tissue formation incidental to the tissue injury imparted by the mechanical dilation, to maximize patency of the sinus drainage pathways post-DCB dilation. Because the mode of transportation was by local diffusion, little drug reached non-target tissue or systemic circulation. The PK cohort of Airiver's FIH clinical trial confirmed that the amount of drug entering the blood was orders of magnitude less than that measured in use of similar vascular DCBs.

### 2.6. RESTORE-1 FIH Clinical Data.

Airiver has completed enrollment in the first in human (FIH), RESTORE-1 Clinical Trial to evaluate the safety and potential efficacy of the Airiver Drug-Coated Balloon in the treatment of Chronic Rhinosinusitis. The study enrolled 45 subjects, treated by five surgeons at two OUS clinical sites, Asuncion, Paraguay and Panama City, Panama.

### 2.6.1. RESTORE-1 STUDY SYNOPSIS.

A synopsis of the RESTORE-1 study is given in Table 24.

TABLE 24
RESTORE-1 Study Synopsis.

| Title: | RESTORE-1: Prospective Trial of Paclitaxel-Coated Nasal Balloon for | |
|---|---|---|
| | the Treatment of Recurrent Chronic Rhinosinusitis With or Without | |
| | Nasal Polyps | |
| Study Description: | Paclitaxel coated balloon is designed to offer both mechanical dilation of | |
| | nasal obstruction and local drug effect for underlying inflammatory | |
| | disorder and cell hyperplasia. | |
| | It is hypothesized that the use of the Airiver balloon in conjunction with | |
| | standard of care will improve patient outcome and with superior nasal | |
| | patency compared to the standard of care alone. This is a prospective, | |
| | single arm, non-randomized first in human (FIH) study | |
| Objectives: | To evaluate safety, and potential efficacy of Airiver Nasal DCB in the | |
| | FIH treatment of recurrent CRSwNP or CRSsNP | |
| Endpoints: | Primary Endpoint | |
| | Primary safety: Freedom from Serious Adverse Events (SAE) | |
| | involving the nasal tissue response through 30-day post-procedure | |
| | Primary efficacy: Freedom from target lesion reintervention due to | |
| | recurrence of CRSsNP or CRSwNP (returning to baseline symptoms or | |
| | worse) through 6 months | |
| | Secondary Endpoints: | |
| | Incidence of, and time to symptom-driven reintervention through a | |
| | minimum of 12 months | |
| | Change in patient reported 22-item sino-nasal outcome test (SNOT- | |
| | 22) from the baseline through a minimum of 12 months | |
| | Change in Lund-Kennedy Endoscopic Scores from the baseline | |
| | through a minimum of 12 months | |
| | Change in Lund-Mackay computed tomography (LMK-CT) score | |
| | from the baseline through a minimum of 12 months. | |
| | Change of Health-related quality -of- life (HRQL) from the baseline | |
| | through a minimum of 12 months (Euro-QOL-5D questionnaire) | |
| | Change in sense of smell using the University of Pennsylvania Smell | |
| | Identification Test (UPSIT) from the baseline through a minimum of | |
| | 12 months | |
| | Clinical pharmacokinetics of paclitaxel (Cmax, Tmax, AUC, T-½) | |
| | in 15 subjects.by 10 days | |
| | Change in Asthma Control Questionnaire-6 (ACQ-5) Scores from | |
| | baseline through a minimum of 12 months for subjects with | |
| | comorbid asthma | |
| | Nasal DCB performance evaluation in index procedures | |
| Study Population: | Up to 45 CRS subjects with recurrent symptomatic nasal obstruction | |
| | eligible for Airiver Nasal DCB treatment | |
| | Target at least 15 subjects with CRSsNP and at least 25 subjects with | |
| | CRSwNP | |
| | Inclusion Criteria: | |
| | 1) | Males or females, ≥18 years |
| | 2) | Signed written informed consent |
| | 3) | Recurrent, symptomatic CRS (with or without nasal polyps, with |
| | | or without prior sinus surgery), have: |
| | | Moderate or severe nasal congestion/blockage/obstruction |
| | | AND decreased or loss of smell (hyposmia or anosmia), |
| | | Or rhinorrhea (anterior/posterior) |
| | | For recurrent CRSwNP: |
| | a) | candidates for RESS or other treatment due to recurrent |
| | | symptom, and endoscopically confirmed present with unilateral |
| | | or bilateral polyps, and/or unilateral or bilateral mucosal disease |
| | | confirmed by nasal endoscopy and/or CT |
| | b) | bilateral sinonasal polyposis that despite prior treatment with |
| | | systemic corticosteroids (SCS) anytime within the past 2 years; |
| | | and/or had a medical contraindication / intolerance to SCS |
| | c) | with or without Aspirin-Exacerbated Respiratory Disease |
| | | (AERD) |
| | | For recurrent CRSsNP: refractory to optimal medical treatment |
| | | and/or previous surgery with positive CT scan of the sinuses |
| | | mucosal thickening and obstruction or positive nasal endoscopic |
| | | finding (purulence or edema) |
| | 4) | Acute Exacerbation of CRS (AECRS) |
| | 5) | Subjects with comorbid asthma or COPD must be stable with no |
| | | exacerbations (e.g., no emergency room visits, hospitalizations) |
| | | for 6 months before the screening visit |
| | | Exclusion Criteria: |
| | 1) | Pediatric CRS (PCRS) |
| | 2) | Acute bacterial sinusitis (ABRS), acute rhinosinusitis (ARS), or |
| | | mycetoma and invasive fungal sinusitis |
| | 3) | Malignancy |
| | 4) | Experienced a cerebrospinal fluid (CSF) leak in prior skull-based |
| | | dehiscence |
| | 5) | Symptomatic without positive CT findings or an asymptomatic |
| | 6) | Subjects whose symptoms are too severe (e.g., temperature > |
| | | 102.58 F. or extrasinus manifestations, such as orbital cellulitis; |
| | | dental or facial or brain abscess; cavernous vein thrombosis; or |
| | | altered mental status |
| | 7) | Primary ciliary dyskinesia (PCD) |
| | 8) | Unable to have nasal cavity examination due to septal deviation |
| | | or spur. Participants who had a sinonasal or sinus surgery |
| | | changing the lateral wall structure of the nose making impossible |
| | | the evaluation of NP |
| | 9) | Have evidence of significant baseline mucosal injury, ulceration, |
| | | or erosion (e.g., exposed cartilage, perforation) on baseline nasal |
| | | examination |
| | 10) | Purulent nasal infection, or upper respiratory tract infection |
| | | within 2 weeks before the screening visit. Potential subjects |
| | | presenting with any of these infections may be rescreened 4 |
| | | weeks after symptom resolution |
| | 11) | Allergy or hypersensitivity to any excipients and paclitaxel. |
| | 12) | Patient has an inability to tolerate endoscopy |
| | 13) | Suffering or recovering from Covid-19 (Fully recovered Covid- |
| | | 19 patients is not excluded) |
| | 14) | Study subject has any disease or condition that interferes with |
| | | safe completion of the study, such as severe COPD or severe |
| | | asthma |
| | 15) | Subjects with abnormal screening laboratory/imaging test results |
| | | that compromise the ability to assess the benefits/risks (e.g., |
| | | abnormal ECG) |
| | 16) | Pregnancy or planning on pregnant during the first 12 months of |
| | | enrollment in the study |
| | 17) | Life expectancy <1 year |
| | 18) | Patient is currently enrolled in other current investigational |
| | | studies. Participation in studies for products approved in the US |
| | | are not considered investigational. |
| | 19) | Lack of informed consent |
| | 20) | Allergic granulomatous angiitis (Churg-Strauss syndrome), |
| | | granulomatosis with polyangiitis (Wegener's granulomatosis), |
| | | Young's syndrome, Kartagener's syndrome or other dyskinetic |
| | | ciliary syndromes |

| | |
|---|---|
| Phase | 1 |
| Description of | Airiver Nasal Drug Coated Balloon (DCB) catheter. The balloon is coated |
| Study Intervention | with a paclitaxel drug (3.5 ug/mm²). |
| Study Duration: | Office visit: 7 days, 30 days, 3 months, 6 months, and 12 months. |
| | Telephone follow-up: annual at 2 years, 3 years, 4 years, and 5 years. |

### 2.6.2. RESTORE-1 Baseline Characteristics.

All 45 symptomatic subjects were diagnosed with CRSwNP or CRSsNP by endoscopy and CT. They all received ESS and DCB treatments in a hospital setting. Patient baseline demographics and clinical characteristics are summarized in Table 25. The balloon sizes used in this Part are summarized in Table 26.

TABLE 25
Patient demographics and baseline characteristics (N = 45).

| Gender, n/N (%) | | |
|---|---|---|
| Male: | 21 | (21/45; 46.7) |
| Female | 24 | (24/45, 53.3) |
| Age (years), | | |
| Mean ± SD | 45.0 ± 14.8 | |
| Median (minimum, maximum) | 45.0 | (18, 73) |
| Duration of disease (year) | | |
| Mean ± SD | 8.2 ± 10.2 | |
| Median (minimum, maximum) | 5.0 | (0.5, 50) |
| CRSsNP, n (%) | 12 | (26.7) |
| CRSwNP, n (%) | 33 | (73.3) |
| Nasal polyps, n (%) | bilateral: 21(63.6); | |
| | single side: 12(36.4) | |
| Nasal endoscopy LK score, | | |
| Mean + SD | 6.59 ± 2.93 | |
| Median (minimum, maximum) | 7 | (0, 12) |
| Nasal CT LM score | | |
| Mean ± SD | 12.76 ± 6.79 | |
| Median (minimum, maximum) | 11 | (1, 24) |
| SNOT-22 score | | |
| Mean ± SD | 50.1 ± 21.9 | |
| Median (minimum, maximum) | 49 | (0, 90) |
| UPSIT score | | |
| Mean ± SD | 19.9 ± 9.49 | |
| Median (minimum, maximum) | 19 | (5, 36) |
| Current smoker, n (%) | 1 | (2.2) |
| Concurrent asthma, n (%) | 9 | (20.0) |
| History of allergy | 3 | (6.7) |
| History of AERD | 1 | (2.2) |
| Prior ESS history n(%) | 12 | (26.7) |
| Number of Balloons Used per Patient | 5.9 | (1-9) |
| mean (min-max) | | |

TABLE 26
Balloon Size Used.

| Balloon Size | Number Used n (%) |
|---|---|
| 10 × 20 mm | 51 (19%) |
| 12 × 20 mm | 52 (19%) |
| 15 × 20 mm | 96 (36%) |
| 18 × 20 mm | 68 (25%) |
| Total | 267 (100%) |

Table 27 details the approximate number of subjects with each sinus type treated, and the total number of sinuses of each type treated with DCB in the RESTORE-1 clinical trial.

TABLE 27
RESTORE-1 Sinuses Treated

| | Approximate Number of |
|---|---|
| | Sinuses n / Number |
| Sinus Treated | of Subjects N |
| Maxillary | 83/44 |
| Anterior Ethmoid | 75/43 |
| Posterior Ethmoid +/− Sphenoid * | 58/33 |
| Frontal | 49/26 |

*Sphenoid sinuses were frequently accessed surgically. The surgical antrostomy was often further dilated coincident with DCB dilation of posterior ethmoid sinuses, as the tip of the DCB catheter was typically positioned within the sphenoid sinus during posterior ethmoid dilation.

### 2.6.3. RESTORE-1 PK Cohort.

15 Subjects participated in the Pharmacokinetic (PK) cohort of the RESTORE-1 trial. Blood draws were taken at baseline and pre-specified intervals following treatment for 10 days. The samples were analyzed with a validated method. Paclitaxel was only measurable in 3/15 subjects at any time point. For the 3 subjects with measurable paclitaxel, only 1 subject had measurable levels on day 3 and paclitaxel was not measurable in any subjects by day 7. The 15 subjects in the PK cohort were treated with a total of 69 DCBs.

### 2.6.4. RESTORE-1 Preliminary Efficacy Results.

### 2.6.4.1. Primary Efficacy Results.

The primary efficacy endpoint was freedom from target lesion reintervention due to recurrence of CRSsNP or CRSwNP (returning to baseline symptoms or worse) through 6-months. There was a 95% freedom from protocol defined target lesion reintervention through 6-months.

Of 44 subjects followed through 6-months, one subject had target lesion reintervention with symptoms returning to baseline or worse. Subject 02-014 had baseline SNOT-22 score of 25 and 6-month SNOT-22 score of 28, with endoscopic observation of polyp recurrence at the 6-month follow-up. They subsequently underwent target lesion reintervention.

A second subject (02-003) underwent target lesion reintervention after observation of polyp recurrence at the 6-month follow up, despite having symptomatic improvement at the 6-month timepoint (Baseline SNOT-22=90, 6-mo SNOT-22=70). Although subject 02-003 did not return to baseline symptoms or worse, a SNOT-22 score of 70 represents significant symptoms. This was considered a target lesion reintervention for calculation of primary efficacy, therefore the freedom from target lesion reintervention was 95% (42/44).

### 2.6.4.2. Additional Efficacy Results.

According to EPOS2020 guidance, the primary goal of treatment of CRS is to achieve and maintain clinical control of the disease in which the patient does not have symptoms, or symptoms are not impacting quality of the life. In the RESTORE-1 trial, patients' nasal symptom severity and impact on patient quality of life were measured by disease-specific SNOT-22 questionnaire, and the impact on patients' general quality of life was measured by EQ-5D-VAS questionnaires.

### Patient Reported Outcomes: SNOT-22, EQ5D-VAS

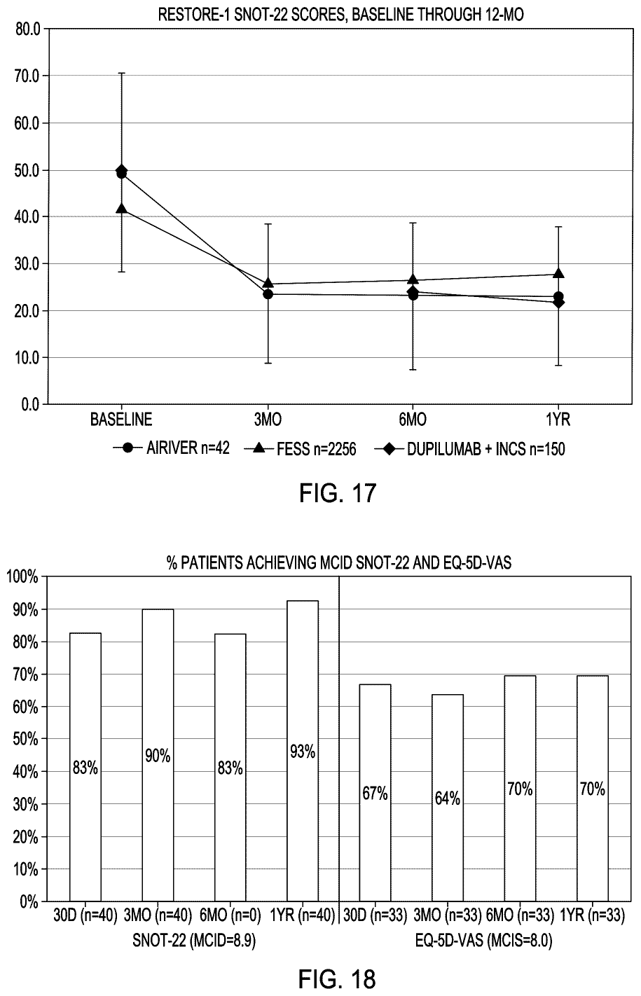

1-year secondary efficacy was analyzed in 32 subjects, based on subjects' reported quality of life improvements. SNOT-22 scores for these subjects are shown in FIG. 17. An 8.9 point change was considered clinically meaningful. For 32 subjects followed through 12-months, the Airiver DCB procedure showed a clinically meaningful mean change from baseline (CFBL) of 27.8 points at 6 months which was sustained through 12 months with mean CFBL=28.9 points. Published data from a biologic therapy trial and a large study of FESS patients are included for reference. The RESTORE-1 data aligned comparably with published FESS data and suggest durable improvement of symptoms through 1-year post-procedure.

The percentage of subjects achieving MCID improvement in SNOT-22 and EQ-5D-VAS at timepoints 30-days through 1-year post-procedure are shown in FIG. 18. Two 1-year subject with baseline SNOT-22 score <8.9 and nine 1-year subjects with baseline EQ-5D-VAS >92 were excluded because improvement by MCID was not possible in these subjects. In a more conservative analysis including these subjects (42 subjects at 1-year), 88% and 55% achieved MCID improvement in SNOT-22 and EQ-5D-VAS respectively. For comparison, in a study of 423 FESS patients with mean pre-op SNOT-22 of 48.9, 79.7% (338/423) achieved SNOT-22 MCID 12-months post-operatively.

The percentage of subjects reporting clinically important improvements in sinus symptoms and general health was greater at 12-months post procedure than at any earlier time-point, suggesting durability of the intervention results.

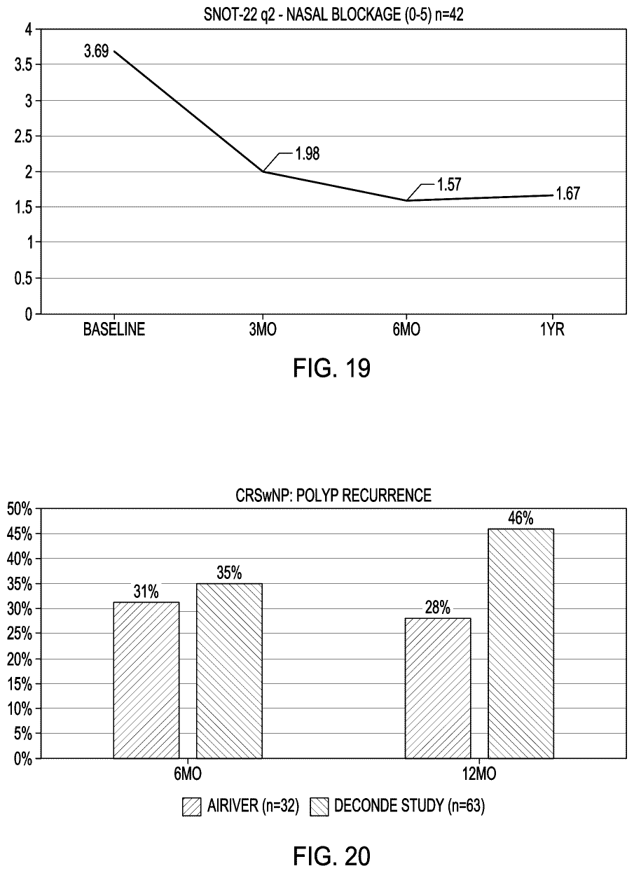

Nasal congestion is a cardinal symptom of CRS. FIG. 19 shows the SNOT-22 Nasal Blockage (nasal congestion) score for 32 RESTORE-1 subjects through 12 months. The data shows a mean 1.71 point (46%) improvement from baseline to 3 months, further improving to a mean 2.02 point (55%) improvement at 12 months, suggesting durable improvement in nasal congestion symptoms through 12 months.

The RESTORE-1 trial assessed polyp recurrence using the Lund-Kennedy endoscopic scoring system. The scoring system is described in Table 28.

TABLE 28
Lund-Kennedy endoscopic grading system.

| Characteristic | Score definition |
|---|---|
| Nasal polyps | 0 = none; 1 = confined to middle meatus; |
| | 2 = beyond middle meatus |
| Discharge | 0 = none; 1 = clear and thin; |
| | 2 = thick and purulent |
| Edema | 0 = absent; 1 = mild; 2 = severe |
| Scarring | 0 = absent; 1 = mild; 2 = severe |
| Crusting | 0 = absent; 1 = mild; 2 = severe |

Thirty-three (33) clinical subjects in the RESTORE-1 trial were identified as having polyps (L-K nasal polyp score >0 on at least one side). One CRSwNP subject was lost to follow-up after 3 months, leaving 32 subjects available for endoscopic evaluation at 6-month and 12-month follow-up timepoints. Presence of polyps at the 6-month and 12-month endoscopies are shown in FIG. 20 compared to published data for FESS alone. The 12-month Airiver data are conservatively presented, as 2 subjects with polyps observed at 6-months underwent revision FESS and polypectomy after their 6-month follow-ups. Both subjects were shown as having polyps at the 12-month time-point, even though 1 subjects was polyp-free.

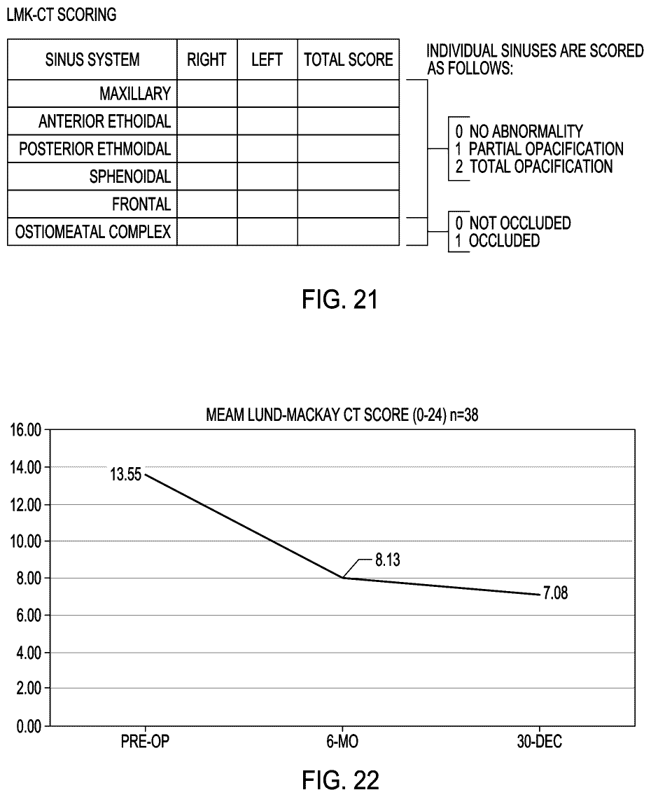

The RESTORE-1 trial assessed Lund-Mackay CT (LMK-CT) score at baseline, 6-months, and 1-year. The Lund-Mackay CT score assessed radiographic evidence of inflammation in each sinus and the ostiomeatal complex (OMC) for each side as shown in FIG. 21, with a possible total LMK-CT score range of 0-24.

Twenty-eight (28) subjects received LMK-CT scores at baseline, 6-months, and 12-months post-procedure. Mean scores from these 38 subjects improved by a mean of 5.4 points at 6-months, and by 6.47 points at 12-months, shown in FIG. 22.

The data indicates radiographic improvement 6-months post procedure that continued through 12-months post-procedure.

### Preliminary PK Results.

Paclitaxel concentrations were measured at various timepoints in treated nasal tissue, plasma, brain tissue, and downstream lung tissue after treatment with multiple DCBs, including overlapping treatments. Residual drug remaining on each treatment DCB from the PK cohort of the study, as a percent of label claim, was analyzed. The characteristics of the PK cohort animals, the total label claim (LC) dose delivered, and residual % LC are reported in Table 29.

Preliminary organ and nasal tissue PK data are summarized in Table 30 and Table 31, and indicate that most drug was delivered to the target mucosal tissue and very little paclitaxel reached downstream or adjacent organs and was cleared quickly.

TABLE 29
GLP Animal Study: PK Cohort.

| | | | | | Total | | |
|---|---|---|---|---|---|---|---|
| | | | | | LC dose | | |
| | Time | Body | | DCB | on 12 | | |
| Animal | point | weight | | treated | DCB | Concentration | Residual |
| number | (day) | (kg) | Gender | n (sizes) | (mg) | mg/kg | % LC |
| 24S0185 | 0.04 | 53.5 | M | All 6 | 71.5 | 1.34 | 66.1 |
| 24S0176 | 7 | 53 | F | sheep | 71.5 | 1.35 | 53.5 |
| 24S0187 | 14 | 51.8 | M | with the | 71.5 | 1.38 | 86.0 |
| 24S0082 | 28 | 39.6 | F | same Tx | 71.5 | 1.81 | 76.7 |
| 24S0192 | 90 | 55.1 | F | matrix: | 71.5 | 1.30 | 73.1 |
| 24S0188 | 90 | 51.2 | M | 12 DCB/ | 71.5 | 1.40 | 72.0 |
| | | | | sheep, | | | |
| | | | | 18 × 20 (9), | | | |
| | | | | 15 × 20 (3) | | | |
| Mean ± SD | na | 50.7 ± 5.1 | na | 12 | 71.5 | 1.43 ± 0.17 | 71.23 ± 9.94 |

TABLE 30
GLP Animal Organ Tissue PK.

| | Brain, 8 samples /animal | | Lung, 8 samples /animal | |
|---|---|---|---|---|
| | | Concentration | | Concentration |
| Time | Sample n with | (ng/g) | Sample n with | (ng/g) |
| point | measurable | mean ± SD | measurable | mean ± SD |
| (day) | level/total | (min,max) | level/total | (min,max) |
| 0.04 | 8/8 | 0.932 ± 0.497 | 8/8 | 23.50 ± 9.436 |
| | | (0.347, 1.71) | | (5.91, 34.0) |
| 7 | 0/8 | 0 | 8/8 | 0.496 ± 0.474 |
| | | | | (0.19, 1.62) |
| 14 | 1/8 | 0.564 (na) | 2/8 | 0.165 ± 0.036 |
| | | | | (0.139, 0.190) |
| 28 | 0/8 | 0 | 0/8 | 0 |
| 90 | 0/16 | 0 | 0/16 | 0 |
| Total sample N | 48 | na | 48 | na |

0 = BLQ(<0.100 ng/g)

TABLE 31
GLP animal Nasal tissue PK.

| | 4 single DCB Tx | | 4 Overlap Tx | | Total 8 Nasal sites treated | |
|---|---|---|---|---|---|---|
| | in each animal | | in each animal | | with 12 DCB per animal | |
| | Sample n | Concentration | Sample n | Concentration | Total sample | Concentration |
| Time | with | (ng/g) | with | (ng/g) | n per time point | (ng/g) per total |
| point | measurable | mean ± SD | measurable | mean ± SD | with measurable | mean ± SD |
| (day) | level/total | (min, max) | level/total | (min, max) | level/total | (min, max) |
| 0.04 | 4/4 | 10063.75 ± 3218.23 | 4/4 | 160585 ± 184999 | 8/8 | 85324.4 ± 145597.8 |
| | | (383, 23700) | | (1640, 427000) | | (383, 427000) |
| 7 | 4/4 | 1605.95 ± 232.18 | 4/4 | 3021.85 ± 4739.87 | 8/8 | 2313.9 ± 3716.32 |
| | | (20.1, 5950) | | (56.4, 10100) | | (20.2, 10100) |
| 14 | 3/3 | 9.0 ± 11.4 | 4/4 | 832.66 ± 1422.60 | 7/7* | 479.66 ± 1098.08 |
| | | (1.25, 22.1) | | (1.21, 2950) | | (1.21, 2950) |
| 28 | 3/4 | 21.27 ± 27.45 | 4/4 | 72.73 ± 91.55 | 7/8 | 50.68 ± 72.10 |
| | | (0, 52.4) | | (4.16, 201) | | (0, 201) |
| 90 | 8/8 | 341.41 ± 553.30 | 7/8 | 117.62 ± 190.40 | 15/16 | 236.97 ± 423.72 |
| | | (7.69, 1390) | | (0, 502) | | (0, 1390) |

*One sample had insufficient sample volume (ISV) for analysis. 0^{∞} =< 0.2BLQ

Scoring for all N=18 Day-7 samples is detailed in Table 32.

TABLE 32
Scoring for Day-7 SEM Samples.

| Score | n/N (%) | Score Description |
|---|---|---|
| 4-0 | 10/18 (55%) | >90% area with ciliated epithelia coverage and |
| | | no mucus/extracellular debris |
| 4-1 | 5/18 (28%) | >90% area with ciliated epithelia coverage and |
| | | little mucus/extracellular debris |
| 4-2 | 3/18 (17%) | >90% area with ciliated epithelia coverage and |
| | | abundant mucus/extracellular debris |

Scoring for all N=18 Day-90 samples is detailed in Table 33.

TABLE 33
Scoring for Day-90 SEM Samples.

| Score | n/N (%) | Score Description |
|---|---|---|
| 4-0 | 10/18 (55%) | >90% area with ciliated epithelia coverage and |
| | | no mucus/extracellular debris |
| 4-1 | 8/18 (45%) | >90% area with ciliated epithelia coverage and |
| | | little mucus/extracellular debris |

### Part IV.

### Non-Vascular Balloon Dilation Device, System, and Treatment Method.

The device described in this Part is a balloon catheter with an instrument access port in the distal portion of the device. An instrument with a preconfigured tip shape can be inserted into the access port and used to direct the device to a target treatment location. The target treatment location could be a paranasal sinus, a paranasal sinus ostium, a paranasal sinus drainage pathway, a partially or completely surgically resected ethmoid sinus cavity, or a eustachian tube. The balloon may be at least partially coated with a therapeutic drug.

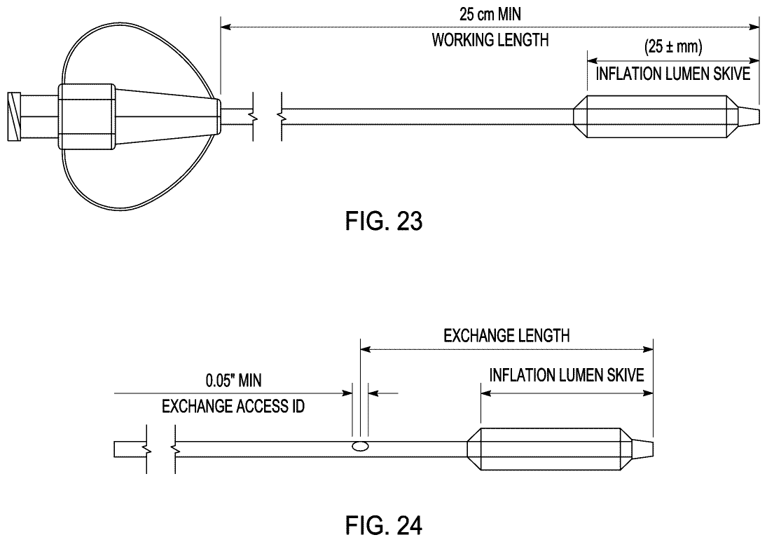

The balloon catheter has a distal tip, an inflatable balloon, a shaft, an instrument insertion port, and a fluid input connection for inflation. A schematic representation of the device is shown in FIG. 23.

An instrument insertion port is located in the distal portion of the device, but proximal to the balloon. The instrument insertion port allows an instrument to be inserted into and through the distal end of the device. The instrument insertion port is shown at the location of “Exchange Access ID” in FIG. 24.

The balloon diameter (OD) when inflated will range from 5 mm to 20 mm. The balloon body length (the cylindrical portion between the tapered cones) will range from 6 mm to 35 mm.

The distal cone angle will range from 60 degrees to 270 degrees. For distal cone angles >180 degrees, the effect will be an inverted distal cone, such that the body of the balloon may extend beyond the tip of the catheter.

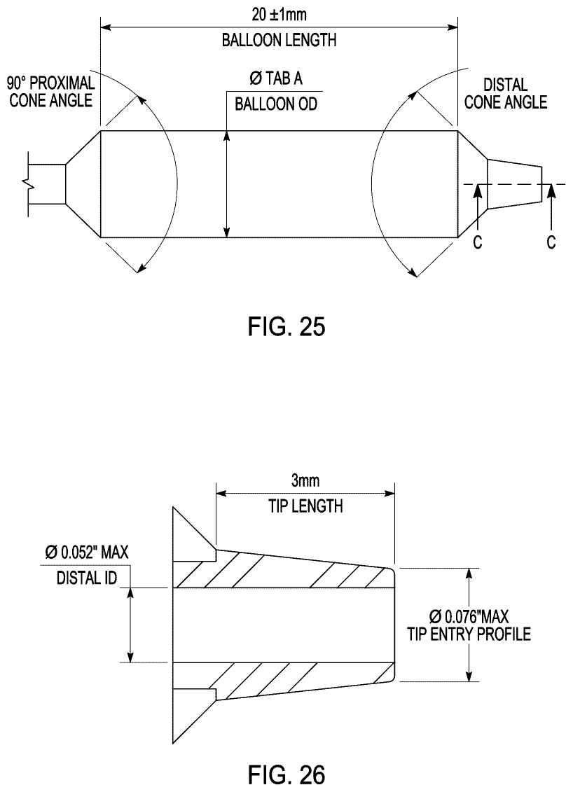

The balloon material may be substantially non-compliant such that the diameter of the inflated balloon changes minimally (<10%) with increasing inflation pressure, up to the maximum labeled inflation pressure. The balloon material may be semi-compliant such that the balloon diameter increases slightly (10%-30%) with increasing inflation pressure, up to the maximum labeled inflation pressure. The balloon is pleated and crimped down to a low profile along the shaft to facilitate easy advancement of the device through constricted anatomy. A detailed schematic of the balloon is shown in FIG. 25.

The tip of the device is tapered and/or radiused to facilitate advancing the distal end of the device through anatomical restrictions such as paranasal sinus ostia, sinus drainage passageways, or eustachian tubes. The tip may be open to allow an instrument to pass through it or may be at least partially enclosed such that the instrument can be inserted but not passed through the distal tip of the device. A detailed view of the open version of the tip is shown in FIG. 26, which shows section C-C from FIG. 25.

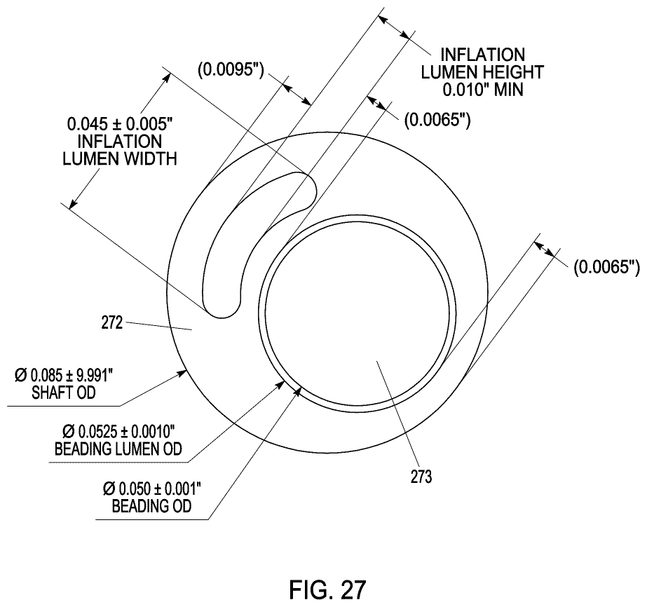

Proximal to the instrument insertion port, the main catheter shaft is a dual lumen design with an integrated inflation lumen. A stiffening member may be included in the large lumen. In the preferred version of the device, the large primary lumen includes a stiffening member made from the same material as the dual lumen catheter shaft. It extends from the fluid input port to the proximal edge of the instrument insertion port and is fused into place at both ends. A section view of the proximal catheter shaft showing the dual lumen tubing 272, the inflation lumen, and the stiffening member 273 are shown in FIG. 27.

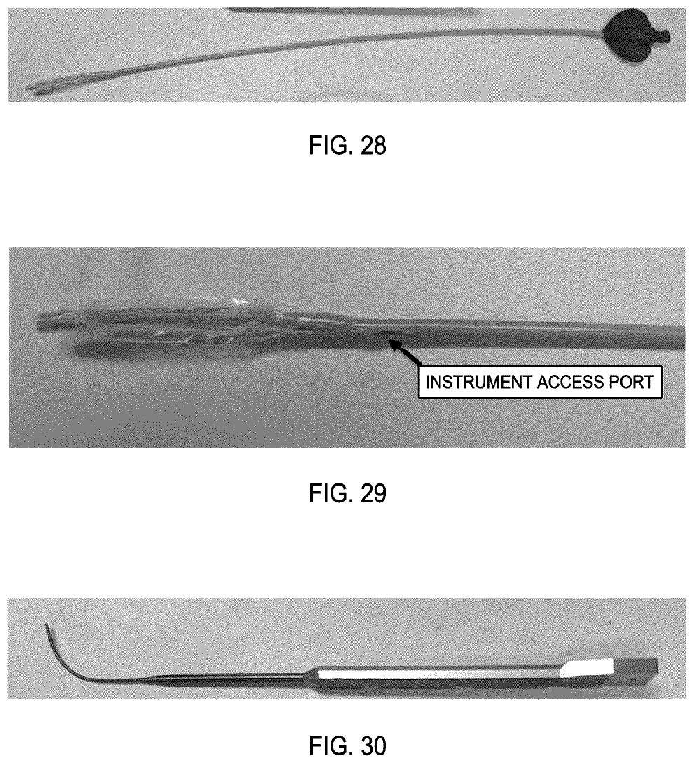

An image of the catheter as manufactured is shown in FIG. 28.

An image of the distal portion of the device showing the instrument access port is shown in FIG. 29.

The shaft color (light green) was chosen to create contrast with the shadow of the instrument access port, to simplify identification of the port location when inserting an instrument during use. Alternative designs include printed or fused markers to identify the location of the instrument access port.

Additional markings may be included on the balloon or on the catheter shaft to indicate the relative position of the device within the anatomy. One such version would be a visible marker located 2 cm proximal to the distal end of the device.

Sensors may be fused into the distal portion of the device such that the coordinates and/or orientation of the device within a 3-dimensional space could be detected using an externally applied magnetic field, for use with image guided surgery systems (IGS).

An example of an instrument that may be used with the balloon catheter is an Airiver sinus seeker as shown in FIG. 30.

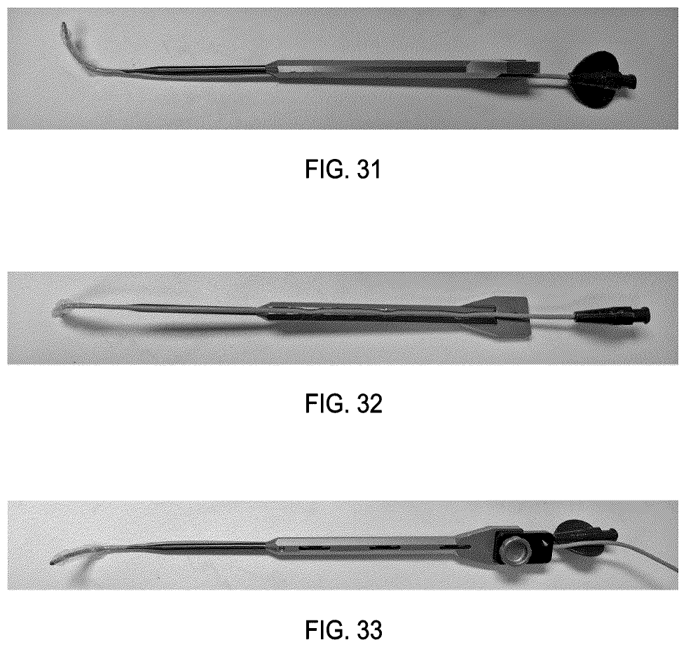

The sinus seeker is shown inserted into the balloon catheter device in FIG. 31.

In the case of the Airiver sinus seeker, the proximal catheter shaft engages with the seeker handle such that the balloon catheter is substantially integrated into the seeker. The seeker includes a longitudinal channel that houses the catheter shaft. The channel includes detent features to maintain the catheter shaft in place during use. The detent features may be “overhangs” such as those shown in FIG. 32 which maintain the radial position of the shaft, but which may allow axial sliding of the shaft, or they may be “nubs” or other press-fit features that restrict radial and axial motion of the catheter shaft. In instances where axial movement of the catheter relative to the sinus seeker is desired, a “button” or other feature may be integrated or attached to the catheter shaft to allow the user to slide the catheter relative to the seeker.

The Seeker includes considerations for attaching sensors for use with IGS systems. An IGS sensor is shown attached by means of a threaded thumb-screw to the proximal end of the seeker in FIG. 33. Alternative versions of the IGS sensor may be clamped to the handle.

The seeker tips may be permanently attached to the seeker or may be interchangeable. FIG. 33 shows an interchangeable tip, held in place by a set screw.

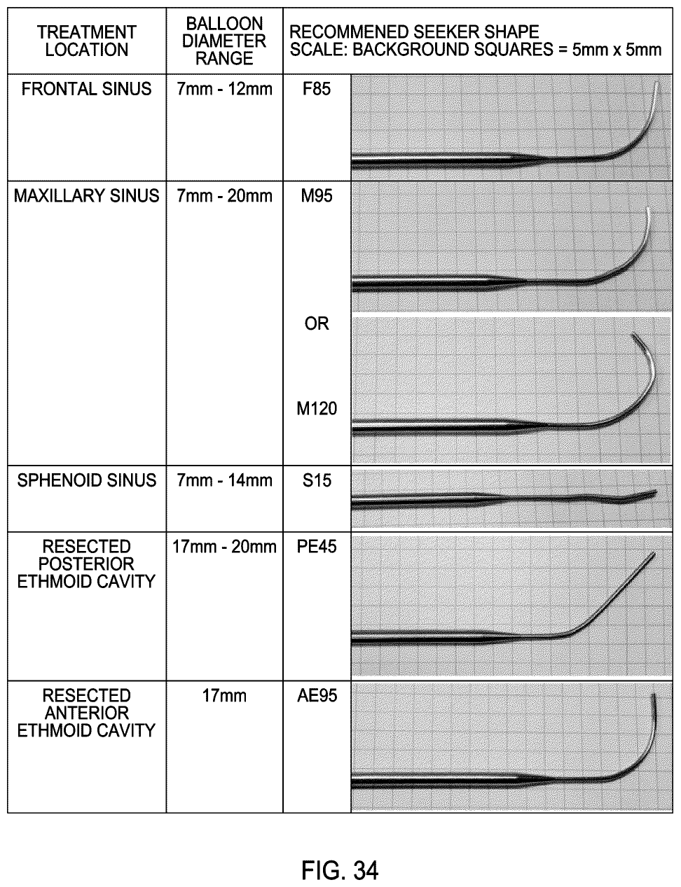

Pre-configured seeker tip shapes facilitate access to various treatment locations. Some examples of preconfigured tip shapes are shown in FIG. 34.

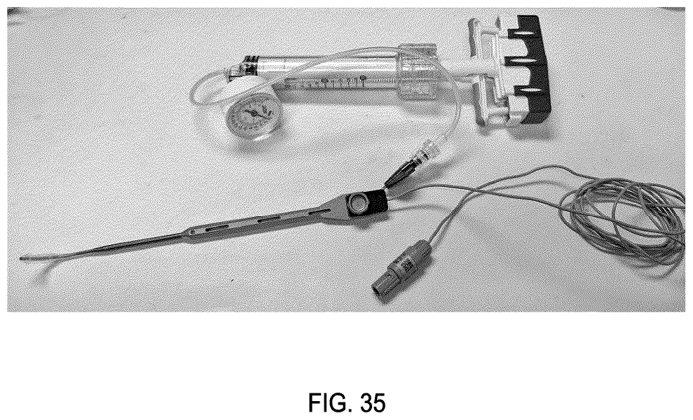

An entire system including the balloon catheter, sinus seeker instrument, IGS sensor, and inflation device are shown in FIG. 35. A simpler version of the system would not include the IGS sensor. The simplest version of the system includes just the balloon catheter and the inflation device.

Example device/system usage: 1) Select the seeker tip configuration to best access the target treatment location. 2) Insert the seeker tip into the access port proximal to the balloon. 3) Press the balloon catheter shaft into the channel in the seeker device. 4) Prepare an inflation device with saline or water. 5) Attach the inflation device to the inflation port on the catheter. 6) For drug coated balloons, soak the balloon in water or saline to hydrate the coating. 7) If desired, attach an IGS sensor to the seeker handle, either by threading it into the handle or by clamping it onto the handle. Calibrate the sensor with the IGS system. 8) Use the seeker to direct the catheter to the target treatment location under endoscopic visualization. Verify the location on the IGS system if applicable. 9) Use the inflation device to inflate the balloon to the desired pressure. 10) Maintain the balloon at the desired pressure for some time (2-10 seconds typical for uncoated balloons, 2-3 minutes typical for drug coated balloons). 11) Deflate the balloon. 12) Withdraw the balloon and positioning instrument.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### EXEMPLARY ASPECTS

The following exemplary aspects are provided, the numbering of which is not to be construed as designating levels of importance:

Aspect 1 provides a method of treatment of a recurring airway stricture or stenosis in an airway body lumen, the method comprising:
inserting a scope and a balloon catheter into a target site at the recurring airway stricture or stenosis in the airway body lumen, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site.

Aspect 2 provides the method of Aspect 1, further comprising damaging, dilating, and/or removing the stricture or stenosis at the target site in the body lumen prior to the insertion of the scope and balloon catheter into the target site.

Aspect 3 provides the method of Aspect 2, wherein the damaging, dilating, and/or removing of the stricture or stenosis inserting a predilation balloon into the body lumen at the target site, inflating the predilation balloon, and removing the predilation balloon prior to inserting the drug coated balloon catheter.

Aspect 4 provides the method of any one of Aspects 2-3, wherein the damaging, dilating, and/or removing comprises surgical removal, electrocautery, laser ablation, cryoablation, radiofrequency ablation, mechanical debulking, rigid bronchoscopy dilation, knife-cutting, direct vision internal stricturotomy, use of an uncoated balloon to dilate the stricture or stenosis, or a combination thereof.

Aspect 5 provides the method of any one of Aspects 1-4, further comprising, prior to the inflating,
flushing the target site with a flushing composition comprising water and/or saline, and hydrating and/or soaking the coating in the flushing composition at the target site, or
hydrating and/or soaking the coating in saline, water, and/or natural fluids native to the body lumen that are not externally added, wherein the hydrating and/or soaking is performed outside the body, in the body lumen while the balloon catheter is en route to the target site, at the target site, or a combination thereof, or
a combination thereof.

Aspect 6 provides the method of Aspect 5 wherein the hydrating and/or soaking is performed for 0.1 minutes to 5 minutes.

Aspect 7 provides the method of any one of Aspects 5-6, wherein the flushing is performed prior to and/or during the insertion of the scope and balloon catheter into the target site.

Aspect 8 provides the method of any one of Aspects 5-7, further comprising
flushing the target site with a flushing composition comprising water and/or saline, and performing the hydrating and/or soaking of the coating in the flushing composition at the target site; or
performing the hydrating and/or soaking of the drug coating outside the body with a flushing composition comprising water and/or saline prior to inserting the drug coated balloon into the target site, and optionally wherein the method is free of flushing at the target site.

Aspect 9 provides the method of any one of Aspects 1-8, wherein the one or more additives comprise one or more water insoluble additives.

Aspect 10 provides the method of any one of Aspects 1-9, wherein the one or more additives comprise one or more slightly water insoluble and/or partially water insoluble additives.

Aspect 11 provides the method of any one of Aspects 1-10, wherein the one or more additives comprise one or more water soluble additives.

Aspect 12 provides the method of any one of Aspects 1-11, wherein the one or more additives comprise:
one or more water soluble additives, and
one or more water insoluble or partially water insoluble additives.

Aspect 13 provides the method of any one of Aspects 1-12, wherein the one or more additives are chosen from pentaerythritol ethoxylate, pentaerythritol propoxylate, pentaerythritol propoxylate/ethoxylate, N-acetylglucosamine, N-octyl-D-gluconamide, N-nonanoyl-N-methylglycamine, N-octanoyl-N-methyl glutamine, C6-ceramide, dihydro-C6-ceramide, cerabroside, sphingomyelin, galaclocerebrosides, lactocerebrosides, N-acetyl-D-sphingosine, N-hexanoyl-D-sphingosine, N-octonoyl-D-sphingosine, N-lauroyl-D-sphingosine, N-palmitoyl-D-sphingosine, sphingosine, polyethylene glycol (PEG) caprylic/capric diglycerides, PEG-8 caprylic/capric glycerides, PEG caprylate, PEG-8 caprylate, PEG caprate, PEG caproate, glyceryl monocaprylate, glyceryl monocaprate, glyceryl monocaproate, monolaurin, monocaprin, monocaprylin, monomyristin, monopalmitolein, monoolein, creatine, creatinine, agmatine, citrulline, guanidine, sucralose, aspartame, hypoxanthine, theobromine, theophylline, adenine, uracil, uridine, guanine, thymine, thymidine, xanthine, xanthosine, xanthosine monophosphate, caffeine, allantoin, (2-hydroxyethyl) urea, N,N′-bis(hydroxymethyl) urea, glycerol ethoxylate, glycerol propoxylate, trimethylolpropane ethoxylate, pentaerythritol, dipentaerythritol, crown ether, 18-crown-6,15-crown-5,12-crown-4, butylated hydroxytoluene (BHT), butylated hydroxyanisole (BHA), a derivative thereof, and combinations thereof.

Aspect 14 provides the method of any one of Aspects 1-13, wherein the one or more additives comprise pentaerythritol ethoxylate, pentaerythritol propoxylate, or a combination thereof.

Aspect 15 provides the method of any one of Aspects 1-14, wherein the balloon has a main diameter that is a nominal inflated diameter of at least 1 mm, or at least 15 mm, or at least 20 mm, or at least 30 mm, or at least 35 mm.

Aspect 16 provides the method of any one of Aspects 1-15, wherein the balloon catheter comprises the elongated balloon and is free of other balloons.

Aspect 17 provides the method of any one of Aspects 1-16, wherein the balloon catheter comprises more than one balloon.

Aspect 18 provides the method of any one of Aspects 1-17, wherein the balloon is substantially free of neck sections between a proximal and a distal end of the balloon.

Aspect 19 provides the method of any one of Aspects 1-18, wherein the balloon comprises at least one neck section on the balloon comprising a smaller diameter than a main diameter of the balloon when the balloon is inflated, the at least one neck section dividing the balloon into at least two main sections each having a diameter.

Aspect 20 provides the method of Aspect 19, wherein the diameter of the at least two main sections is equal to the main diameter of the elongated balloon, or the at least one neck section has a diameter that is about 5% to about 99% of the diameter of at least one of the at least two main sections.

Aspect 21 provides the method of any one of Aspects 19-20, wherein the at least one neck section has a diameter that is independently about 5 mm to about 40 mm.

Aspect 22 provides the method of any one of Aspects 19-21, wherein the diameter of the at least one neck section is substantially static during inflation of the balloon.

Aspect 23 provides the method of any one of Aspects 19-22, wherein the at least one neck section comprises a substantially nonelastic portion of the balloon, a reinforced portion of the balloon, or a combination thereof.

Aspect 24 provides the method of any one of Aspects 19-23, wherein the at least one neck section comprises an inelastic material around a circumference of the neck section.

Aspect 25 provides the method of Aspect 24, wherein the inelastic material comprises ultra high molecular weight polyethylene, a nylon, a polyamide, or a combination thereof

Aspect 26 provides the method of any one of Aspects 19-25, wherein the diameter (e.g., inflated diameter) of the at least two main sections are about 5 mm to about 45 mm.

Aspect 27 provides the method of any one of Aspects 19-26, wherein the at least one neck section is about 1% to about 50% of the balloon length.

Aspect 28 provides the method of any one of Aspects 19-27, wherein the at least one neck section is one neck section and the balloon is free of other neck sections.

Aspect 29 provides the method of any one of Aspects 19-28, wherein the at least one neck section is two neck sections and the balloon is free of other neck sections.

Aspect 30 provides the method of Aspect 29, wherein the two neck sections have about the same diameter.

Aspect 31 provides the method of any one of Aspects 29-30, wherein one of the two neck sections have a smaller diameter than the other neck section.

Aspect 32 provides the method of any one of Aspects 29-31, wherein the two neck sections are symmetrically located with respect to the center of the balloon length.

Aspect 33 provides the method of any one of Aspects 29-32, wherein the balloon catheter comprises three of the main sections separated by the two neck sections.

Aspect 34 provides the method of any one of Aspects 29-33, wherein the at least one neck section is three neck sections, wherein the balloon is free of other neck sections.

Aspect 35 provides the method of Aspect 34, wherein the three neck sections are arranged to provide four of the main sections separated by the three neck sections.

Aspect 36 provides the method of any one of Aspects 1-35, wherein the elongated balloon has a length of about 20 mm to about 300 mm.

Aspect 37 provides the method of any one of Aspects 1-36, wherein the balloon catheter comprises a catheter shaft on a longitudinal end of the balloon, the catheter shaft comprising an interior lumen for delivery of a gas, liquid, or a combination thereof, to the balloon interior.

Aspect 38 provides the method of any one of Aspects 1-37, wherein the balloon catheter comprises an atraumatic Coude tip.

Aspect 39 provides the method of any one of Aspects 1-38, wherein after the deflation and withdrawal from the target site the balloon catheter comprises a residual drug amount that is less than 100 wt % of the initial drug load.

Aspect 40 provides the method of any one of Aspects 1-39, wherein after the deflation and withdrawal from the target site the balloon catheter comprises a residual drug amount that is about 70 wt % or less of the initial drug load.

Aspect 41 provides the method of any one of Aspects 1-40, wherein the initial drug load is from about 1 microgram to about 20 micrograms of the therapeutic agent per square millimeter of the balloon, measured when the balloon is at its nominal inflated diameter.

Aspect 42 provides the method of any one of Aspects 1-41, wherein the initial drug load is from about 2 to about 6 micrograms of the therapeutic agent per square millimeter of the balloon, measured when the balloon is at its nominal inflated diameter.

Aspect 43 provides the method of any one of Aspects 1-42, wherein a ratio by weight of the therapeutic agent in the coating layer to the total weight of the one or more additives in the coating layer is from about 0.05 to about 20.

Aspect 44 provides the method of any one of Aspects 1-43, wherein the ratio by weight of the therapeutic agent in the coating layer to the total weight of the one or more additives in the coating layer is from about 0.5 to about 8.

Aspect 45 provides the method of any one of Aspects 1-44, wherein the ratio by weight of the therapeutic agent in the coating layer to the total weight of the one or more additives in the coating layer is from about 2 to about 6.

Aspect 46 provides the method of any one of Aspects 1-45, wherein the balloon catheter has a stretch ratio of about 1.0 to about 20.

Aspect 47 provides the method of any one of Aspects 1-46, wherein the balloon catheter further comprises a sheath covering the elongated balloon, wherein the method comprises removing the sheath before the inflating.

Aspect 48 provides the method of any one of Aspects 1-47, wherein inserting the scope and the balloon catheter comprises inserting the balloon catheter through a lumen of the scope.

Aspect 49 provides the method of any one of Aspects 1-48, wherein inserting the scope and the balloon catheter comprises inserting the balloon catheter and the scope side-by-side.

Aspect 50 provides the method of any one of Aspects 1-49, comprising placing the scope and a proximal edge of the balloon at or near the target site.

Aspect 51 provides the method of any one of Aspects 1-50, wherein the scope is an endoscope, rhinolaryngoscope, rhinoscope, bronchoscope, cystoscope, or a combination thereof.

Aspect 52 provides the method of any one of Aspects 1-51, comprising visualizing positioning of the balloon catheter at the target site with the scope.

Aspect 53 provides the method of any one of Aspects 1-52, wherein the inflating is performed at least until the target site yields and is dilated.

Aspect 54 provides the method of any one of Aspects 1-53, wherein the inflating is performed such that the ratio of the inflation diameter to a normative body lumen diameter at the target site is about 1.0 to about 20.

Aspect 55 provides the method of any one of Aspects 1-54, wherein the inflating is performed such that the ratio of the inflation diameter to a normative body lumen diameter at the target site is about 1.0, 1.1, 1.2, or 1.31 to 10.

Aspect 56 provides the method of any one of Aspects 1-55, wherein the inflating is performed such that the balloon is inflated to a pressure that is equal to or greater than a nominal pressure of the balloon.

Aspect 57 provides the method of any one of Aspects 1-56, wherein the inflating is performed such that a stretch ratio of the inflated diameter of the balloon to a normative body lumen diameter at the target site is about 1.0 to about 20.

Aspect 58 provides the method of any one of Aspects 1-57, wherein the inflating is performed such that a stretch ratio of the inflated diameter of the balloon to a normative body lumen diameter at the target site is about 1.0, 1.1, 1.2, or 1.31 to 10.

Aspect 59 provides the method of any one of Aspects 1-58, wherein the inflating is performed such that a ratio of the inflation diameter to a normative body lumen diameter at the target site is about 1.0 to 20, and such that a stretch ratio of the inflated diameter of the balloon to a normative body lumen diameter at the target site of about 1.0 to about 20.

Aspect 60 provides the method of any one of Aspects 1-59, wherein the inflating is performed such that the balloon is inflated to a pressure greater than a nominal pressure of the balloon, and a nominal inflated diameter of the balloon is less than the inflated diameter.

Aspect 61 provides the method of any one of Aspects 1-60, wherein the inflating comprises observing pressure within the balloon.

Aspect 62 provides the method of any one of Aspects 1-61, wherein the inflating comprises inflating the balloon to a first pressure, allowing pressure within the balloon to stabilize while maintaining the first pressure in the balloon for a stabilization period, then resuming increasing pressure in the balloon until the inflation diameter is achieved.

Aspect 63 provides the method of Aspect 62, comprising visualizing the yielding and dilation of the target site with the scope.

Aspect 64 provides the method of any one of Aspects 1-63, comprising visualizing the inflating with the scope.

Aspect 65 provides the method of any one of Aspects 1-64, comprising maintaining the inflation diameter by keeping the balloon inflated for 1 minute to 7 days.

Aspect 66 provides the method of any one of Aspects 1-65, comprising maintaining the inflation diameter by keeping the balloon inflated for 1 minute to 1 day.

Aspect 67 provides the method of any one of Aspects 1-66, comprising maintaining the inflation diameter by keeping the balloon inflated for 1 minute to 10 minutes.

Aspect 68 provides the method of any one of Aspects 1-67, comprising maintaining the inflation diameter for a duration sufficient to release the drug into tissue of the target site and/or to prevent or reduce bleeding.

Aspect 69 provides the method of any one of Aspects 1-68, wherein the body lumen comprises a frontal sinus, ethmoid sinus, sphenoid sinus, maxillary sinus, nasal passage, supraglottis, glottis, subglottis, trachea, mainstem bronchus, bronchus intermedius, lobar bronchus, segmental bronchus, or sub-segmental bronchus.

Aspect 70 provides the method of any one of Aspects 1-69, wherein the recurring stricture or stenosis is idiopathic.

Aspect 71 provides the method of any one of Aspects 1-70, wherein the recurring stricture or stenosis is caused by a congenital condition, trauma, inflammation, post-intubation tracheal stenosis, post-tracheostomy tracheal restenosis, post-tuberculosis infection, transplant-related restenosis, repeated medication treatments, surgical removal, electrocautery, laser ablation, cryoablation, mechanical debulking, rigid bronchoscopic dilation, stent placement, and/or balloon dilation, and/or wherein the stricture or stenosis comprises CRS stenosis, CRSwNP stenosis, nasal stenosis, severe asthma, comorbidities of CRSwNP, comorbidities of severe asthma stenosis, subglottic stricture and/or stenosis, laryngostenosis, tracheal stenosis, bronchial stenosis, airway anastomotic stenosis, radiation induced airway stenosis, bronchial smooth muscle cells, IL-4, IL-5, IL-6, IL-13, IL-23, ILC2, mucus plug, goblet cells, fibrosis, cystic fibrosis, and/or one or more mucins.

Aspect 72 provides the method of any one of Aspects 1-71, wherein the airway stricture or stenosis comprises a tracheal and/or bronchial stricture or stenosis.

Aspect 73 provides the method of any one of Aspects 1-72, wherein the airway stricture or stenosis comprises chronic rhinosinusitis with nasal polyps (CRSwNP).

Aspect 74 provides the method of any one of Aspects 1-73, wherein the airway stricture or stenosis comprises laryngostenosis and/or subglottic stricture or stenosis.

Aspect 75 provides the method of any one of Aspects 1-74, wherein the airway stricture or stenosis is a stricture or stenosis induced by repeated medication treatments, intubation, tracheostomy, tuberculosis infection, surgical removal, electrocautery, laser ablation, cryoablation, mechanical debulking, rigid bronchoscopic dilation, stent placement, and/or balloon dilation.

Aspect 76 provides the method of any one of Aspects 1-75, further comprising damaging, dilating, and/or removing the stricture or stenosis at the target site in the body lumen prior to the insertion of the scope and balloon catheter into the target site, wherein:
the method is a method of treating chronic rhinosinusitis with nasal polyps (CRSwNP), wherein the airway stricture or stenosis is a structure or stenosis induced by CRSwNP.

Aspect 77 provides the method of any one of Aspects 1-76, further comprising damaging, dilating, and/or removing the stricture or stenosis at the target site in the body lumen prior to the insertion of the scope and balloon catheter into the target site, wherein:
the method is a method of treating laryngostenosis and/or subglottic stricture or stenosis, wherein the airway stricture or stenosis is a structure or stenosis induced by laryngostenosis and/or subglottic stricture or stenosis.

Aspect 78 provides the method of any one of Aspects 1-77, wherein the predilation balloon is substantially free of a drug coating.

Aspect 79 provides a method of treatment of a recurring airway stricture or stenosis in an airway body lumen, the method comprising:
damaging, dilating, and/or removing the stricture or stenosis at a target site in the body lumen;
inserting a scope and a balloon catheter into the target site, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
hydrating and/or soaking the coating in the flushing composition at the target site;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 80 provides a method of treatment of chronic rhinosinusitis with nasal polyps (CRSwNP), the method comprising:
removing at least one of the nasal polyps at a target site in the body lumen;
inserting a scope and a balloon catheter into the target site in a body lumen, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 81 provides a method of treatment of laryngostenosis and/or subglottic stricture or stenosis, the method comprising:
damaging, dilating, and/or removing the laryngostenosis and/or subglottic stricture or stenosis at a target site in the body lumen;
inserting a scope and a balloon catheter into the target site in a body lumen, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 82 provides a method of treatment of severe asthma, the method comprising:
inserting a scope and a balloon catheter into a target site in a body lumen comprising a trachea, mainstem bronchus, bronchus intermedius, lobar bronchus, segmental bronchus, or sub-segmental bronchus, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;

inflating the balloon to an inflation diameter such that the coating contacts an interior of the body lumen at the target site;

deflating the balloon; and

withdrawing the scope and the balloon catheter from the target site;

wherein prior to inflation the drug coated balloon is soaked and/or hydrated outside of the body to activate the coating.

Aspect 83 provides a method of reduction of exacerbation of and/or hospitalization for severe asthma, the method comprising:
inserting a scope and a balloon catheter into a target site in a body lumen comprising a trachea, mainstem bronchus, bronchus intermedius, lobar bronchus, segmental bronchus, or sub-segmental bronchus, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated outside the body to activate the coating.

Aspect 84 provides a method of reducing a concentration of bronchial smooth muscle cells of a severe asthma patient, the method comprising:
inserting a scope and a balloon catheter into a target site comprising bronchial smooth muscle cells in a body lumen, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated outside the body to activate the coating.

Aspect 85 provides a method of reducing a concentration of eosinophils, IL-4, IL-5, IL-6, IL-13, IL-23, and/or ILC2 in an airway tract, the method comprising:
inserting a scope and a balloon catheter into a target site in a body lumen comprising a trachea, mainstem bronchus, bronchus intermedius, lobar bronchus, segmental bronchus, or sub-segmental bronchus, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 86 provides a method of treatment of a mucous plug in an airway, the method comprising:
removing mucous plug from a target site in a body lumen;
inserting a scope and a balloon catheter into the target site, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 87 provides a method of reduction of concentration of goblet cells in an airway tract, the method comprising:
inserting a scope and a balloon catheter into a target site comprising one or more goblet cells in a body lumen, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 88 provides a method reducing a concentration of one or more mucins in the airway tract, the method comprising:
inserting a scope and a balloon catheter into a target site in a body lumen comprising a trachea, mainstem bronchus, bronchus intermedius, lobar bronchus, segmental bronchus, or sub-segmental bronchus, wherein the balloon catheter comprises
   an elongated balloon, and
   a coating layer overlying an exterior surface of the balloon, wherein the coating layer comprises one or more additives and an initial drug load of a therapeutic agent chosen from paclitaxel, docetaxel, taxol, rapamycin, sirolimus, zotarolimus, everolimus, tacrolimus, an analogue thereof, and a combination thereof;
inflating the balloon to an inflation diameter such that the coating contacts an interior of the body lumen at the target site;
deflating the balloon; and
withdrawing the scope and the balloon catheter from the target site;
wherein prior to inflation the drug coated balloon is soaked and/or hydrated to activate the coating.

Aspect 89 provides a method of treatment of chronic rhinosinusitis with nasal polyps (CRSwNP) and asthma, the method comprising:
removing at least one of the nasal polyps at a first target site in a first body lumen;
inserting a first scope and a first balloon catheter into the first target site in the first body lumen, wherein the first balloon catheter comprises
   a first elongated balloon, and
   a first coating layer overlying an exterior surface of the first balloon, wherein the first coating layer comprises one or more first additives and an initial drug load of a first therapeutic agent chosen from paclitaxel, docetaxel, taxol, rapamycin, sirolimus, zotarolimus, everolimus, tacrolimus, an analogue thereof, and a combination thereof;
inflating the first balloon to a first inflation diameter such that the first coating layer contacts an interior of the first body lumen at the first target site;
deflating the first balloon;
withdrawing the first scope and the first balloon catheter from the first target site;
inserting a second scope and a second balloon catheter into a second target site in a second body lumen comprising a trachea and/or a bronchus, wherein the second balloon catheter comprises
   a second elongated balloon, and
   a second coating layer overlying an exterior surface of the second balloon, wherein the second coating layer comprises one or more second additives and an initial drug load of a second therapeutic agent chosen from paclitaxel, docetaxel, taxol, rapamycin, sirolimus, zotarolimus, everolimus, tacrolimus, an analogue thereof, and a combination thereof;
inflating the second balloon to an inflation diameter such that the coating contacts an interior of the second body lumen at the second target site;
deflating the second balloon;
withdrawing the second scope and the second balloon catheter from the target site;
optionally performing the treatment again at a third target site in a trachea and/or bronchus that is different than the second target site;
wherein prior to inflation or the first and/or second drug coated ballon, the first and/or second drug coated balloon is soaked and/or hydrated to activate the coating.

Aspect 90 provides the method of Aspect 89, wherein a time between the treatment of the second target site and the treatment of the third target site is 1 to 6 weeks.

Aspect 91 provides the method of Aspect 89, wherein the second target site comprises a bronchus.

Aspect 92 provides the method of Aspect 91, wherein the bronchus comprises a mainstem bronchus, bronchus intermedius, and/or a lobar bronchus.

Aspect 93 provides the method of any one of Aspects 89-92, wherein the asthma is severe asthma.

Aspect 94 provides a method of treating a central airway obstruction (CAO), benign airway stenosis, asthma, chronic obstructive pulmonary disease (COPD), or an airway tumor in a human subject, the method comprising:
inserting a drug-coated balloon catheter into a target site in a trachea, mainstem bronchus, bronchus intermedius, lobar bronchus, segmental bronchus, or sub-segmental bronchus;
inflating the balloon to dilate the airway and deliver a therapeutic agent to the airway wall;
deflating the balloon; and
withdrawing the balloon catheter from the target site;
wherein the balloon is coated with a coating layer comprising the therapeutic agent and one or more additives, and the therapeutic agent comprises paclitaxel, sirolimus, or a derivative thereof.

Aspect 95 provides the method of Aspect 94, wherein the balloon catheter is delivered through the working channel of a bronchoscope.

Aspect 96 provides the method of Aspect 94 or 95, wherein the balloon catheter is an over-the-wire catheter compatible with a guidewire (e.g., a 0.035″ guidewire).

Aspect 97 provides the method of any of Aspects 94-96, wherein the balloon has a nominal diameter of 4 mm to 20 mm and a length of 30 mm to 65 mm.

Aspect 98 provides the method of Aspect 97, wherein the balloon has a nominal diameter of 8 mm, 10 mm, 12 mm, 14 mm, 15 mm, or 18 mm.

Aspect 99 provides the method of any of Aspects 94-98, wherein the balloon is inflated to a nominal pressure of 2 atm to 12 atm.

Aspect 100 provides the method of Aspect 99, wherein the balloon is inflated to a nominal pressure of 3 atm to 6 atm.

Aspect 101 provides the method of any of Aspects 94-100, wherein the coating layer comprises the therapeutic agent at a dose density of 1 μg/mm² to 20 μg/mm².

Aspect 102 provides the method of Aspect 101, wherein the dose density is 3.5 μg/mm².

Aspect 103 provides the method of any of Aspects 94-102, wherein the one or more additives comprise pentaerythritol ethoxylate (PEE).

Aspect 104 provides the method of any of Aspects 94-103, wherein the weight ratio of the one or more additives to the therapeutic agent in the coating is from 0.01:1 to 10:1.

Aspect 105 provides the method of Aspect 104, wherein the weight ratio of the one or more additives to the therapeutic agent is from 0.1:1 to 0.5:1.

Aspect 106 provides the method of any of Aspects 94-105, wherein the balloon catheter comprises two radiopaque marker bands indicating the drug-coated working length.

Aspect 107 provides the method of any of Aspects 94-106, wherein the balloon catheter is provided sterile and is intended for single use.

Aspect 108 provides the method of any of Aspects 94-107, wherein the balloon is soaked in saline or water for 0.1 to 5 minutes prior to insertion to hydrate the coating.

Aspect 109 provides the method of Aspect 108, wherein the balloon is soaked for 1 to 2 minutes.

Aspect 110 provides the method of any of Aspects 94-109, wherein the balloon is inflated at the target site for 1 minute to 10 minutes.

Aspect 111 provides the method of Aspect 110, wherein the balloon is inflated for 2 to 3 minutes.

Aspect 112 provides the method of any of Aspects 94-111, wherein the method further comprises monitoring the pressure during inflation and maintaining the balloon at a desired pressure for a predetermined time.

Aspect 113 provides the method of any of Aspects 94-112, wherein the method is performed in a subject with post-intubation tracheal stenosis, post-tracheostomy tracheal stenosis, post-transplantation stenosis, or post-COVID airway stenosis.

Aspect 114 provides the method of any of Aspects 94-113, wherein the method is performed in a subject with a Myer-Cotton grade 2, 3, or 4 airway stenosis.

Aspect 115 provides the method of any of Aspects 94-114, wherein the method results in a reduction in the Myer-Cotton grade of the stenosis by at least one grade at 1 month post-procedure.

Aspect 116 provides the method of any of Aspects 94-115, wherein the method results in a freedom from symptom-driven target lesion reintervention of at least 80% at 6 months post-procedure.

Aspect 117 provides the method of Aspect 116, wherein the method results in a freedom from symptom-driven target lesion reintervention of about 86% to about 90% at 6 months post-procedure.

Aspect 118 provides the method of any of Aspects 94-117, wherein the method results in an improvement in FEV1 of at least 100 mL in about 50% to about 70% of patients at 12 months post-procedure.

Aspect 119 provides the method of Aspect 118, wherein the method results in an improvement in FEV1 of at least 100 mL in about 68% of patients at 12 months post-procedure.

Aspect 120 provides the method of any of Aspects 94-119, wherein the method results in an improvement in mMRC dyspnea score of at least 1 grade in about 75% to about 90% of patients at 12 months post-procedure.

Aspect 121 provides the method of Aspect 120, wherein the method results in an improvement in mMRC dyspnea score of at least 1 grade in about 85% of patients at 12 months post-procedure.

Aspect 122 provides the method of any of Aspects 94-121, wherein the method results in an improvement in Peak Expiratory Flow of greater than 18.8 L/min in about 85% to about 95% of patients at 12 months post-procedure.

Aspect 123 provides the method of Aspect 122, wherein the method results in an improvement in Peak Expiratory Flow of greater than 18.8 L/min in about 92.9% of patients at 12 months post-procedure.

Aspect 124 provides a method of treating chronic rhinosinusitis (CRS) or chronic rhinosinusitis with nasal polyps (CRSwNP) in a human subject, the method comprising:
identifying a target lesion in a paranasal sinus or sinus drainage pathway;
optionally performing surgical dissection, irrigation, or balloon dilation with an uncoated balloon;
inserting a drug-coated balloon catheter into the target lesion;
inflating the balloon to dilate the lesion and deliver a therapeutic agent to the tissue;
deflating the balloon; and
withdrawing the balloon catheter;
wherein the balloon is coated with a coating layer comprising the therapeutic agent and one or more additives, wherein the therapeutic agent comprises paclitaxel, sirolimus, or a derivative thereof.

Aspect 125 provides the method of Aspect 124, wherein the balloon is selected to have a nominal inflated diameter 1 mm to 10 mm larger than the prepared lesion.

Aspect 126 provides the method of Aspect 125, wherein the balloon is selected to have a nominal inflated diameter 2 mm to 3 mm larger than the prepared lesion.

Aspect 127 provides the method of any of Aspects 124-126, wherein the balloon is soaked in saline or water for 0.1 to 5 minutes prior to insertion.

Aspect 128 provides the method of Aspect 127, wherein the balloon is soaked for 1 to 2 minutes.

Aspect 129 provides the method of any of Aspects 124-128, wherein the balloon is inflated at the target site for 1 minute to 10 minutes.

Aspect 130 provides the method of Aspect 129, wherein the balloon is inflated for 2 minutes.

Aspect 131 provides the method of any of Aspects 124-130, wherein the balloon is a rapid exchange catheter with a semi-compliant sinus dilation balloon.

Aspect 132 provides the method of any of Aspects 124-131, wherein the balloon has a labeled diameter of 7 mm to 20 mm and a length of 20 mm to 35 mm.

Aspect 133 provides the method of Aspect 132, wherein the balloon has a labeled diameter of 10 mm, 12 mm, 14 mm, 15 mm, 17 mm, or 20 mm.

Aspect 134 provides the method of any of Aspects 124-133, wherein the balloon is coated with the therapeutic agent at a dose density of 1 μg/mm² to 20 μg/mm².

Aspect 135 provides the method of Aspect 134, wherein the dose density is 3.5 μg/mm².

Aspect 136 provides the method of any of Aspects 124-135, wherein the one or more additives comprise pentaerythritol ethoxylate (PEE).

Aspect 137 provides the method of any of Aspects 124-136, wherein the weight ratio of the one or more additives to the therapeutic agent in the coating is from 0.01:1 to 10:1.

Aspect 138 provides the method of Aspect 137, wherein the weight ratio is from 0.1:1 to 0.5:1.

Aspect 139 provides the method of any of Aspects 124-138, wherein the balloon is coated on the cylindrical area between the balloon cones, and optionally on the distal cone.

Aspect 140 provides the method of any of Aspects 124-139, wherein the method results in a freedom from target lesion reintervention due to recurrence of CRS or CRSwNP of at least 90% at 6 months post-procedure.

Aspect 141 provides the method of Aspect 140, wherein the method results in a freedom from target lesion reintervention of about 95% at 6 months post-procedure.

Aspect 142 provides the method of any of Aspects 124-141, wherein the method results in a mean change from baseline in SNOT-22 score of at least 8.9 points at 12 months post-procedure.

Aspect 143 provides the method of Aspect 142, wherein the method results in a mean change from baseline in SNOT-22 score of at least 27 points at 12 months post-procedure.

Aspect 144 provides the method of any of Aspects 124-143, wherein the method results in a mean change from baseline in Lund-Mackay CT score of at least 5 points at 12 months post-procedure.

Aspect 145 provides the method of any of Aspects 124-144, wherein the therapeutic agent is measurable in blood of fewer than 25% of patients at any time point following treatment.

Aspect 146 provides the method of any of Aspects 124-144, wherein the therapeutic agent is not measurable in blood of any patients by day 7 following treatment.

Aspect 147 provides a method of reshaping and flattening out residual bony partitions in a surgically resected ethmoid sinus cavity, the method comprising:
performing surgical ethmoidectomy to create a resected ethmoid cavity;
inserting a balloon catheter into the resected ethmoid cavity;
inflating the balloon to reshape and flatten residual bony partitions;
deflating and removing the balloon.

Aspect 148 provides the method of Aspect 147, wherein the balloon is a drug-coated balloon as recited in any of Aspects 31-46.

Aspect 149 provides the method of Aspect 147 or 148, wherein the balloon is inserted and inflated at least 1 day after ethmoidectomy.

Aspect 150 provides the method of Aspect 149, wherein the balloon is inserted and inflated 1 to 30 days after ethmoidectomy.

Aspect 151 provides a method of treating a eustachian tube disorder in a human subject, the method comprising:
inserting a balloon catheter into the eustachian tube;
inflating the balloon to dilate the eustachian tube and deliver a therapeutic agent to the tissue;
deflating the balloon; and
withdrawing the balloon catheter;
wherein the balloon is coated with a coating layer comprising the therapeutic agent and one or more additives, wherein the therapeutic agent comprises paclitaxel, sirolimus, or a derivative thereof.

Aspect 152 provides the method of Aspect 151, wherein the balloon has a diameter of 2 mm to 10 mm and a length of 10 mm to 30 mm.

Aspect 153 provides the method of Aspect 151 or 152, wherein the balloon is inflated for 1 minute to 10 minutes.

Aspect 154 provides the method of any of Aspects 151-153, wherein the method results in improved eustachian tube function as measured by tympanometry or patient-reported outcomes.

Aspect 155 provides a device for treating nasal sinuses or paranasal sinus drainage pathways, comprising:
a tubular balloon catheter with an integrated inflation lumen;
a hollow region under the balloon; and
an access port in the distal portion of the device but proximal to the balloon, through which a positioning instrument may be inserted.

Aspect 156 provides the device of Aspect 155, wherein the balloon is pleated and crimped.

Aspect 157 provides the device of Aspect 155 or 156, wherein the device has a closed distal tip.

Aspect 158 provides the device of any of Aspects 155-157, further comprising one or more sensors fused or embedded into the distal portion of the device for use with an image guidance system (IGS).

Aspect 159 provides the device of any of Aspects 155-158, wherein the balloon is coated with a coating comprising a therapeutic drug on at least a portion of the balloon.

Aspect 160 provides the device of Aspect 159, wherein the therapeutic drug comprises a therapeutic agent comprising paclitaxel, sirolimus, or a derivative thereof, and wherein the coating further comprises one or more additives.

Aspect 161 provides the device of any of Aspects 155-160, wherein the access port is visually marked or color-contrasted for identification.

Aspect 162 provides the device of any of Aspects 155-161, wherein the device further comprises markings on the balloon or catheter shaft to indicate the relative position of the device within the anatomy.

Aspect 163 provides the device of any of Aspects 155-162, wherein the balloon has a diameter when inflated of about 5 mm to about 20 mm and a body length of about 6 mm to about 35 mm.

Aspect 164 provides the device of any of Aspects 155-163, wherein the device comprises a dual lumen design with an integrated inflation lumen and a stiffening member.

Aspect 165 provides the device of Aspect 164, wherein the stiffening member extends from a fluid input port to a proximal edge of the instrument insertion port.

Aspect 166 provides a system comprising:
a balloon dilation device as recited in any of Aspects 155-165; and
a positioning instrument configured to be inserted into the balloon device through the access port in the distal portion of the device but proximal to the balloon.

Aspect 167 provides the system of Aspect 166, further comprising an inflation device.

Aspect 168 provides the system of Aspect 166 or 167, further comprising a navigation system.

Aspect 169 provides the system of any of Aspects 166-168, wherein the positioning instrument comprises a sinus seeker with a preconfigured tip shape.

Aspect 170 provides the system of Aspect 169, wherein the sinus seeker is integrated with the balloon catheter by a channel with detent features to maintain the catheter shaft in place during use.

Aspect 171 provides the system of Aspect 169 or 170, wherein the sinus seeker includes an IGS sensor attached to a handle of the sinus seeker.

Aspect 172 provides the system of any of Aspects 166-171, wherein the balloon is coated with a coating comprising a therapeutic drug.

Aspect 173 provides the system of Aspect 172, wherein the therapeutic drug comprises a therapeutic agent comprising paclitaxel, sirolimus, or a derivative thereof, and wherein the coating further comprises one or more additives.

Aspect 174 provides the system of any of Aspects 166-173, wherein the positioning instrument includes interchangeable tips secured by a set screw or threaded attachment.

Aspect 175 provides a method of positioning a balloon catheter in a nasal cavity, paranasal sinus, or sinus drainage pathway, comprising:
inserting a positioning instrument into a port in the distal end of the balloon catheter, proximal to the balloon; and
positioning the balloon catheter and positioning instrument into the nasal cavity and directing the balloon catheter to the target location.

Aspect 176 provides the method of Aspect 175, wherein the method is performed under endoscopic visualization.

Aspect 177 provides the method of Aspect 175 or 176, wherein the method further comprises confirming the location using an image guidance system (IGS), wherein an IGS sensor is located in the distal part of the balloon catheter or connected to the positioning instrument.

Aspect 178 provides the method of any of Aspects 175-177, wherein the balloon catheter is as recited in any of Aspects 155-165.

Aspect 179 provides the method of any of Aspects 175-178, wherein the balloon is coated with a coating comprising a therapeutic drug.

Aspect 180 provides the method of Aspect 179, wherein the therapeutic drug comprises a therapeutic agent comprising paclitaxel, sirolimus, or a derivative thereof, and the coating further comprises one or more additives.

Aspect 181 provides the method, device, or system of any one or any combination of Aspects 1-180 optionally configured such that all elements or options recited are available to use or select from.

What is claimed is:

1. A method of treating a central airway obstruction (CAO), benign airway stenosis, asthma, chronic obstructive pulmonary disease (COPD), or an airway tumor in a human subject, the method comprising:
inserting a drug-coated balloon catheter into a target site in a trachea, mainstem bronchus, bronchus intermedius, lobar bronchus, segmental bronchus, or sub-segmental bronchus;
inflating the balloon to dilate the airway and deliver a therapeutic agent to the airway wall;
deflating the balloon; and
withdrawing the balloon catheter from the target site;
wherein the balloon is coated with a coating layer comprising the therapeutic agent and one or more additives, wherein the therapeutic agent comprises paclitaxel, sirolimus, or a derivative thereof.

2. The method of claim 1, wherein the balloon has a nominal diameter of 4 mm to 20 mm and a length of 30 mm to 65 mm.

3. The method of claim 1, wherein the coating layer comprises the therapeutic agent at a dose density of 1 μg/mm² to 20 μg/mm², the one or more additives comprise pentaerythritol ethoxylate (PEE), and the weight ratio of the one or more additives to the therapeutic agent in the coating is from 0.01:1 to 10:1.

4. The method of claim 1, wherein the balloon is soaked in saline or water for 0.1 to 5 minutes prior to insertion to hydrate the coating.

5. The method of claim 1, wherein the balloon is inflated at the target site for 1 minute to 10 minutes.

6. The method of claim 1, wherein the method results in:
a reduction in the Myer-Cotton grade of the stenosis by at least one grade at 1 month post-procedure,
a freedom from symptom-driven target lesion reintervention of at least 80% at 6 months post-procedure,
an improvement in FEV1 of at least 100 mL in about 50% to about 70% of patients at 12 months post-procedure,
an improvement in mMRC dyspnea score of at least 1 grade in about 75% to about 90% of patients at 12 months post-procedure,
an improvement in Peak Expiratory Flow of greater than 18.8 L/min in about 85% to about 95% of patients at 12 months post-procedure, or
a combination thereof.

7. A method of treating chronic rhinosinusitis (CRS) or chronic rhinosinusitis with nasal polyps (CRSwNP) in a human subject, the method comprising:
identifying a target lesion in a paranasal sinus or sinus drainage pathway;
optionally performing at the target lesion surgical dissection, irrigation, or balloon dilation with an uncoated balloon;
inserting a drug-coated balloon catheter into the target lesion, wherein the balloon is coated with a coating layer comprising a therapeutic agent and one or more additives, wherein the therapeutic agent comprises paclitaxel, sirolimus, or a derivative thereof;
inflating the balloon to dilate the lesion and deliver the therapeutic agent to the tissue;
deflating the balloon; and
withdrawing the balloon catheter.

8. The method of claim 7, wherein the balloon is soaked in saline or water for 0.1 to 5 minutes prior to insertion.

9. The method of claim 7, wherein the balloon is inflated at the target site for 1 minute to 10 minutes.

10. The method of claim 7, wherein the balloon has a labeled diameter of 7 mm to 20 mm and a length of 20 mm to 35 mm.

11. The method of claim 7, wherein the balloon is coated with the therapeutic agent at a dose density of 1 μg/mm² to 20 μg/mm², wherein the one or more additives comprise pentaerythritol ethoxylate (PEE), and wherein the weight ratio of the one or more additives to therapeutic agent in the coating is from 0.01:1 to 10:1.

12. The method of claim 7, wherein the method results in:
a freedom from target lesion reintervention due to recurrence of CRS or CRSwNP of at least 90% at 6 months post-procedure, or
a mean change from baseline in SNOT-22 score of at least 8.9 points at 12 months post-procedure, or
a mean change from baseline in Lund-Mackay CT score of at least 5 points at 12 months post-procedure, or
a combination thereof.

13. The method of claim 7, wherein the therapeutic agent is measurable in blood of fewer than 25% of patients at any time point following treatment.

14. The method of claim 7, wherein the method comprises:
performing surgical ethmoidectomy to create a resected ethmoid cavity;
inserting the balloon catheter into the resected ethmoid cavity;
inflating the balloon to reshape and flatten residual bony partitions; and
deflating and removing the balloon.

15. A method of treating a eustachian tube disorder in a human subject, the method comprising:
inserting a balloon catheter into the eustachian tube, wherein the balloon is coated with a coating layer comprising a therapeutic agent and one or more additives;
inflating the balloon to dilate the eustachian tube and deliver the therapeutic agent to the tissue;
deflating the balloon; and
withdrawing the balloon catheter.

16. A system comprising:
a balloon dilation device comprising
a tubular balloon catheter with an integrated inflation lumen,
a hollow region under the balloon, and
an access port in the distal portion of the device but proximal to the balloon, through which a positioning instrument may be inserted; and
a positioning instrument configured to be inserted into the balloon device through the access port in the distal portion of the device but proximal to the balloon.

17. The system of claim 16, wherein the positioning instrument comprises a sinus seeker with a preconfigured tip shape.

18. The system of claim 16, wherein the balloon is coated with a coating comprising a therapeutic drug comprising paclitaxel, sirolimus, or a derivative thereof, and wherein the coating further comprises one or more additives.

19. The system of claim 16, wherein the positioning instrument includes interchangeable tips secured by a set screw or threaded attachment.

20. A method of positioning the balloon dilation device of the system of claim 16 in a nasal cavity, paranasal sinus, or sinus drainage pathway, comprising:
inserting the positioning instrument into the port in the distal end of the balloon dilation device; and
positioning the balloon catheter and positioning instrument into the nasal cavity and directing the balloon catheter to the target location.
